SEQUENCE LISTING (26 sequences)

SEQ ID NO: 1 - RNA, 120 nt, Artificial Sequence
  Description of Artificial Sequence Synthetic oligonucleotide
gccuacggccacaccacccugaaagugccugaucucgucugaucucagaagcgauacagggucgggccugguuaguaccgugaugggagaccgccugggaauaccaggugucguaggcuu

SEQ ID NO: 2 - RNA, 56 nt, Artificial Sequence
  Description of Artificial Sequence Synthetic oligonucleotide
aacuaaaacaauuuuugaagaacaguuucuguacuucauugguauguagagacuuc

SEQ ID NO: 3 - RNA, 132 nt, Artificial Sequence
  Description of Artificial Sequence Synthetic oligonucleotide
ucaaggccgccugagugcgguuuuaccgcauaccaauaacgcuucacucgaggcguuuuucguuauguauaaauaaggagcacaccaugcaauaugccauugcaggguggccuguugcuggcugcccuuccg

SEQ ID NO: 4 - DNA, 20 bp, Artificial Sequence
  Description of Artificial Sequence Synthetic oligonucleotide
gtcatagctgtttcctgtgt

SEQ ID NO: 5 - DNA, 20 bp, Artificial Sequence
  Description of Artificial Sequence Synthetic oligonucleotide
gttgggtaacgccagggttt

SEQ ID NO: 6 - DNA, 20 bp, Artificial Sequence
  Description of Artificial Sequence Synthetic oligonucleotide
cgcttctggtgccggaaacc

SEQ ID NO: 7 - DNA, 20 bp, Artificial Sequence
  Description of Artificial Sequence Synthetic oligonucleotide
atgcgctcaggtcaaattca

SEQ ID NO: 8 - DNA, 20 bp, Artificial Sequence
  Description of Artificial Sequence Synthetic oligonucleotide
cggaaaatgccgctcatccg

SEQ ID NO: 9 - DNA, 20 bp, Artificial Sequence
  Description of Artificial Sequence Synthetic oligonucleotide
tagagattcgggatttcggc

SEQ ID NO: 10 - DNA, 20 bp, Artificial Sequence
  Description of Artificial Sequence Synthetic oligonucleotide
agttgttctgcttcatcagc

SEQ ID NO: 11 - DNA, 20 bp, Artificial Sequence
  Description of Artificial Sequence Synthetic oligonucleotide
atgccgtgggtttcaatatt

SEQ ID NO: 12 - DNA, 20 bp, Artificial Sequence
  Description of Artificial Sequence Synthetic oligonucleotide
cgggaagggctggtcttcat

SEQ ID NO: 13 - DNA, 20 bp, Artificial Sequence
  Description of Artificial Sequence Synthetic oligonucleotide
gggagcgtcacactgaggtt

SEQ ID NO: 14 - RNA, 17 nt, Artificial Sequence
  Description of Artificial Sequence Synthetic oligonucleotide
gggggaaaaaaaccccc

SEQ ID NO: 15 - RNA, 17 nt, Artificial Sequence
  Description of Artificial Sequence Synthetic oligonucleotide
cccccuuuuuuuggggg

SEQ ID NO: 16 - RNA, 32 nt, Artificial Sequence
  Description of Artificial Sequence Synthetic oligonucleotide
nnnnncugangarnnnnnnnnnnygaaannnn

SEQ ID NO: 17 - RNA, 48 nt, Artificial Sequence
  Description of Artificial Sequence Synthetic oligonucleotide
nnnnngaavnnnnnnnnagaaacannnnnnnnnnnanauuacnnnnnn

SEQ ID NO: 18 - RNA, 21 nt, Homo sapiens
aacgacuauauguguccagcc

SEQ ID NO: 19 - RNA, 21 nt, Artificial Sequence
  Description of Artificial Sequence Synthetic oligonucleotide
cgacuauauguguccagccuu

SEQ ID NO: 20 - RNA, 21 nt, Artificial Sequence
  Description of Artificial Sequence Synthetic oligonucleotide
ggcuggacacauauagucguu

SEQ ID NO: 21 - RNA, 21 nt, Homo sapiens
aaccagugcaccauugauaaa

SEQ ID NO: 22 - RNA, 21 nt, Artificial Sequence
  Description of Artificial Sequence Synthetic oligonucleotide
ccagugcaccauugauaaauu

SEQ ID NO: 23 - RNA, 21 nt, Artificial Sequence
  Description of Artificial Sequence Synthetic oligonucleotide
uuuaucaauggugcacugguu

SEQ ID NO: 24 - RNA, 21 nt, Homo sapiens
aaaugcuacgaagugggaaug

SEQ ID NO: 25 - RNA, 21 nt, Artificial Sequence
  Description of Artificial Sequence Synthetic oligonucleotide
augcuacgaagugggaauguu

SEQ ID NO: 26 - RNA, 21 nt, Artificial Sequence
  Description of Artificial Sequence Synthetic oligonucleotide
cauucccacuucguagcauuu

(19) United States
(12) Patent Application Publication
(10) Pub. No.: US 2004/0002083 A1
(43) Pub. Date: Jan. 1, 2004

(54) Statistical algorithms for folding and target accessibility prediction and design of nucleic acids

(76) Inventors: Ye Ding, Schenectady, NY (US); Charles E. Lawrence, Melrose, NY (US)

Correspondence Address: FROMMER LAWRENCE & HAUG, 745 FIFTH AVENUE- 10TH FL., NEW YORK, NY, 10151, US

(21) Appl. No.: 10/348,935

(22) Filed: Jan. 22, 2003

Related U.S. Application Data
(60) Provisional application No. 60/352,643, filed on Jan. 29, 2002.

(51) Int. Cl. C12Q 1/68; G06F 19/00; G01N 33/48; G01N 33/50

(52) U.S. Cl. 435/6; 702/20

(57) ABSTRACT

A method of predicting structural characteristics of a nucleic acid molecule. A method of predicting single-stranded regions in the secondary structure of a nucleic acid molecule in accordance with a probability distribution of structures based on recursively generated partition functions for the identification of accessible sites on target RNA for gene down-regulation and the rational design of antisense oligos, trans-cleaving ribozymes, siRNAss and antisense RNAs, for interaction with other RNA-targeting molecules, and for rational design of nucleic acid probes such as molecular beacons for RNA or DNA targets.

### RELATED APPLICATIONS/INCORPORATION BY REFERENCE

This application claims priority to U.S. provisional application Serial No. 60/352,643, filed Jan. 29, 2002, incorporated herein by reference.

Indeed, each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference. More generally, documents or references are cited in this text, either in a Reference List before the claims, or in the text itself; and, each of these documents or references (“herein-cited references”), as well as each document or reference cited in each of the herein-cited references (including any manufacturer's specifications, instructions, etc.), is hereby expressly incorporated herein by reference.

### FIELD OF THE INVENTION

The present invention relates to statistical algorithms for predicting structural characteristics of nucleic acid molecules and target accessibility prediction for the rational design of antisense nucleic acids, for evaluating molecular interactions, and for design of nucleic acid probes.

### BACKGROUND OF THE INVENTION

Efficient gene down-regulation methods are of paramount importance for high-throughput functional studies of genes and gene products in humans and model organisms, as well as in infectious pathogens, and for the validation of new therapeutic targets and agents for the treatment of human diseases. Antisense oligonucleotides (oligos) and trans-cleaving ribozymes have been widely used for inhibition of gene expression in both prokaryotes and eukaryotes. It has been recently shown that short interfering RNAs (siRNAs) can also induce gene silencing in mammalian cells through a process known as RNA interference (RNAi). Together, these RNA-targeting have emerged as increasingly important tools for gene modulation. For these antisense nucleic acid molecules to be effective, they must first bind to target messenger RNA (mRNA) or viral RNA in a sequence-specific manner, through complementary base pairing. To a large extent, target accessibility is determined by the secondary structure of the target RNA. Experimental approaches for accessibility evaluation are laborious, time consuming, and expensive. As a result, computational methods for accessibility prediction have been in development.

With respect to accessible site identifying and targeting methods, reference is made to the following:

U.S. Pat. No. 5,780,610 (“the '610 patent”) issued to Collins et al. is directed toward a method for substantially reducing background signals encountered in nucleic acid hybridization assays. The method is premised on the elimination or significant reduction of the phenomenon of non-specific hybridization, so as to provide a detectable signal which is produced only in the presence of the target polynucleotide of interest. As applied to the construction of hybridizing oligonucleotides for antisense compounds, the '610 patent describes the use of short regions of hybridization between multiple probes and a target to reduce nonspecific hybridization with non-target species that result from using conventional antisense molecules.

U.S. Pat. Nos. 5,856,103 and 6,183,966 issued to Gray et al. relate to a system and method for assessing the minimum of RNA:DNA sequence combinations whose properties need to be determined for selecting antisense oligonucleotide sequences that will form the most stable hybrid among all those possible in a given target mRNA sequence. The method further comprises a data processing system for identifying nucleic acid sequences for antisense oligonucleotide targeting. The method uses a control computer that includes a nearest-neighbor nucleic acid pair value data list. The nearest-neighbor nucleic acid pair value data list is determined by referring to a set of predetermined nucleic acid nearest-neighbor bond comparisons. The thermodynamic energies needed for splitting a combination of nearest-neighbor base pairs apart are used to determine the ranking of the nearest-neighbor nucleic acid pairs, and, thus the sequence of priority in which the location of antisense pairing is sought. A target sequence is then received by the computer and analyzed. The computer program uses combinations of nearest-neighbor base pair stabilities, rather than rely on assignments of individual nearest-neighbor base pair stabilities.

Each of these references provides accessible site identifying and targeting features. However, it has been found desirable to be able to determine specific structural characteristics of a target RNA molecule for improved accessible site identification and targeting.

With respect to techniques for determining structural characteristics of an RNA molecule, reference is made to the following:

Zuker, M., On finding all suboptimal foldings of an RNA molecule. Science 244, 48-52 (1989); Zuker, M., The use of dynamic programming algorithms in RNA secondary structure prediction. In Waterman, M. S. (Ed.), Mathematical Methods for DNA Sequences, CRC Press, Boca Raton, Fla., pp. 159-184 (1989); and Zuker, M. and Stiegler, P., Optimal computer folding of large RNA sequences using thermodynamics and auxiliary information. Nucleic Acids Res. 9, 133-148 (1981) describe the so-called mfold algorithm, developed with dynamic programming algorithms, that predicts optimal folding through free energy minimization and presents suboptimal foldings.

These suboptimal foldings have limitations due to algorithmic design, and they do not guarantee a statistically valid sample of probable structures.

Wuchty, S., Fontana, W., Hofacker, I. L., Schuster, P., Complete suboptimal folding of RNA and the stability of secondary structures. Biopolymers 49 (2), 145-65 (1999) proposes complete enumeration of a large number of all possible structures with free energies within a threshold of the global minimum.

This approach is computationally prohibitive for sequences of even moderate length.

McCaskill, J. S., The equilibrium partition function and base pair binding probabilities for RNA secondary structure. Biopolymers 29, 1105-1119 (1990) includes a probabilistic algorithm using the partition function approach that computes base pairing probability and the binding probability for any base. A C program for this algorithm is available in a suite of RNA secondary structure software known as the Vienna RNA package. This package was developed by a theoretical chemistry group at the University of Vienna (Hofacker et al., www.tbi.univie.ac.at/˜ivo/RNA/).

However, the algorithm does not generate any secondary structures.

As such, there is a need for an efficient and statistically unbiased method of predicting structural characteristics of an RNA molecule, in particular an mRNA or viral RNA molecule for antisense nucleic acid applications.

The following are hereby incorporated by reference:

Allawi, H. T., Dong, F., Ip, H. S., Neri, B. P., Lyamichev, V. I. (2001) Mapping of RNA accessible sites by extension of random oligonucleotide libraries with reverse transcriptase. RNA 7, 314-27.

Altuvia, S., Kornitzer, D., Teff, D., Oppenheim, A. B. (1989) Alternative mRNA structures of the cIII gene of bacteriophage lambda determine the rate of its translation initiation. J Mol Biol. 210, 265-80.

Ambros, V. (2001) microRNAs: tiny regulators with great potential. Cell. 107, 82 3-6.

Asano, K., Niimi, T., Yokoyama, S., and Mizobuchi, K. (1998) Structural basis for binding of the plasmid ColIb-P9 antisense Inc RNA to its target RNA with the 5′-rUUGGCG-3′ motif in the loop sequence. J Biol Chem. 273, 11826-11838.

Bennett, C. F., Cowsert, L. M. (1999) Antisense oligonucleotides as a tool for gene functionalization and target validation. Biochim Biophys Acta. 1489 (1), 19-30.

Berzal-Herranz, A., Joseph, S., Chowrira, B. M., Butcher, S. E., Burke, J. M. (1993) Essential nucleotide sequences and secondary structure elements of the hairpin ribozyme. EMBO J 12, 2567-73.

Bonhoeffer, S., McCaskill, J. S., Stadler, P. F. , Schuster, P. (1993) Eur. Biophys. J. 22, 13-24.

Brookes, A. J. (1999). The essence of SNPs. Gene 234 (2), 177-86.

Brower, V. (1998). Genome II: the next frontier. Nat Biotechnol. 16 (11), 1004.

Brown, J. W. (1999) The Ribonuclease P Database. Nucleic Acids Res. 27, 314.

Brown, P. O., Botstein, D. (1999). Exploring the new world of the genome with DNA microarrays. Nat Genet. 21 (1 Suppl), 33-7.

Burgess, T. L., Fisher, E. F., Ross, S. L, Bready, J. V., Qian, Y X, Bayewitch, L. A., Cohen, A. M., Herrera, C. J., Hu, S. S., Kramer, T. B., et al. (1995) The antiproliferative activity of c-myb and c-myc antisense oligonucleotides in smooth muscle cells is caused by a nonantisense mechanism. Proc. Natl. Acad. Sci. USA. 92 (9), 4051-5.

Cazenave, C., Loreau, N., Thuong, N. T., Toulme, J. J., and Helene, C. (1987) Enzymatic amplification of translation inhibition of rabbit beta-globin mRNA mediated by anti-messenger oligodeoxynucleotides covalently linked to intercalating agents. Nucleic Acids Res. 15, 717-4736. (hereinafter “Cazenave et al.”)

Cech, T. R., Zaug, A. J., Grabowski, P. J. (1981) In vitro splicing of the ribosomal RNA precursor of Tetrahymena: involvement of a guanosine nucleotide in the excision of the intervening sequence. Cell 27, 487-96.

Cech, T. R., Damberger, S. H., Gutell, R. R. (1994). Representation of the secondary and tertiary structure of group I introns. Nat. Struct. Biol. 1, 273-80.

Christoffersen, R. E., McSwiggen, J. A., Konings, D. (1994) Application of computational technologies to ribozyme biotechnology products. J. Mol. Structure (Theochem) 311, 273-284. (hereinafter “Christoffersen et al.”).

Comolli, L. R., Pelton, J. G. and Tinoco, I. Jr (1998) Mapping of a protein-RNA kissing hairpin interface: Rom and Tar-Tar*. Nucleic Acids Res. 26, 4688-4695.

Crooke, S. T. (1998) An overview of progress in antisense therapeutics. Antisense Nucleic Acid Drug Dev. 8, 115-22.

Crooke, S. T. (2000) Progress in antisense technology: the end of the beginning. Methods Enzymol. 313, 3-45.

Cupal, J., Flamm, C., Renner, A. and Stadler, P. F. (1997). Density of states, metastable states, and saddle points exploring the energy landscape of an RNA molecule. Proceedings of ISMB97 88-91. (hereinafter “Cupal et al.”)

Dallas, A. and Moore, P. B. (1997) The loop E-loop D region of Escherichia coli 5S rRNA: the solution structure reveals an unusual loop that may be important for binding ribosomal proteins. Structure 5, 1639-53.

Damberger, S. H., Gutell, R. R. (1994) A comparative database of group I intron structures. Nucleic Acids Res. 22, 3508-10.

De Backer, M. D., Nelissen, B., Logghe, M., Viaene, J., Loonen, I, Vandoninck, S., de Hoogt, R., Dewaele, S., Simons, F. A., Verhassel, t P., Vanhoof, G., Contreras, R., Luyten, W. H. (2001) An antisense-based functional genomics approach for identification of genes critical for growth of Candida albicans. Nature Biotechnol. 19, 235-41.

Ding, Y. (2002) Rational statistical design of antisense oligonucleotides for high throughput functional genomics and drug target validation. Statistica Sinica 12, 273-296. (hereinafter “Ding”).

Ding, Y., and Lawrence, C. E. (2001) Statistical prediction of single-stranded regions in RNA secondary structure and application to predicting effective antisense target sites an beyond, Nucleic Acids Res. 29, 1034-1046.

Ding, Y., and Lawrence, C. E. (1999) A Bayesian statistical algorithm for RNA secondary structure prediction. Computers and Chemistry 23, 387-400.

Driver, S. E., Robinson, G. S., Flanagan, J., Shen, W., Smith, L. E., Thomas, D. W., Roberts, P. C. (1999) Oligonucleotide-based inhibition of embryonic gene expression. Nat. Biotechnol. 17 (12), 1184-7.

Easterwood, T. R. and Harvey, S. C. (1997) Ribonuclease P RNA: models of the 15/16 bulge from Escherichia coli and the P15 stem loop of Bacillus subtilis. RNA 3, 577-85.

Eckardt, S., Romby, P., Sczakiel, G. (1997) Implications of RNA structure on the annealing of a potent antisense RNA directed against the human immunodeficiency virus type 1. Biochemistry 36, 12711-21.

Eisen, M. B., Spellman, P. T., Brown, P. O., Botstein, D. (1998) Cluster analysis and display of genome-wide expression patterns. Proc. Natl. Acad. Sci. USA. 95 (25), 14863-8.

Eguchi, Y., Itoh, T., Tomizawa, J. (1991) Antisense RNA. Annu. Rev. Biochem. 60, 631-52.

Elbashir, S. M., Harborth, J., Lendeckel, W., Yalcin, A., Weber, K., Tuschl, T. (2001) Duplexes of 21-nucleotide RNAs mediate RNA interference in cultured mammalian cells. Nature 411, 494-8.

Ferrℑ-D'Amarℑ and A. R., Doudna, J. A. (1999) Annu. Rev. Biophys. Biomol. Struct. 28, 57-73.

Fire, A., Xu, S., Montgomery, M. K., Kostas, S. A., Driver, S. E., Mello, C. C. (1998) Potent and specific genetic interference by double-stranded RNA in Caenorhabditis elegans. Nature. 391, 806-11.

Franch, T., Petersen, M., Wagner, E. G., Jacobsen, J. P., Gerdes, K. (1999) Antisense RNA regulation in prokaryotes: rapid RNA/RNA interaction facilitated by a general U-turn. J. Mol. Biol. 294(5), 1115-25.

Fraser, A. G., Kamath, R. S., Zipperlen, P., Martinez-Campos, M., Sohrmann, M., Ahringer, J. (2000). Functional genomic analysis of C. elegans chromosome I by systematic RNA interference. Nature 408, 325-30.

Gonczy, P., Echeverri, C., Oegema, K., Coulson, A., Jones, S. J., Copley, R. R., Duperon, J., Oegema, J., Brehm, M., Cassin, E., Hannak, E., Kirkham, M., Pichler, S., Flohrs, K., Goessen, A., Leidel, S., Alleaume, A. M., Martin, C., Ozlu, N., Bork, P., Hyman, A. A. (2000) Functional genomic analysis of cell division in C. elegans using RNAi of genes on chromosome III. Nature 408, 331-6.

Goodchild, J., Carrol, E. III and Greenberg, J. R. (1988) Inhibition of human immunodeficiency virus replication by antisense oligodeoxynucleotides. Arch. Biochem. Biophys. 263, 401-409. (hereinafter “Goodchild et al.”)

Guerrier-Takada, C., Altman, S. (1984) Catalytic activity of an RNA molecule prepared by transcription in vitro. Science 223, 285-6.

Gultyaev, A. P., van Batenburg, F. H., Pleij, C. W. (1999) An approximation of loop free energy values of RNA H-pseudoknots. RNA 5, 609-17. (hereinafter “Gultyaev et al.”)

Gultyaev, A. P., van Batenburg, F. H. D. and Pleij, C. W. A. (1995) The computer simulation of RNA folding pathways using a genetic algorithm. J. Mol. Biol. 250, 37-51.

Gutell, R. R. (1994) Collection of small subunit (16S- and 16S-like) ribosomal RNA structures. Nucleic Acids Res. 22, 3502-3507.

Haseloff, J., Gerlach, W. L. (1988) Simple RNA enzymes with new and highly specific endoribonuclease activities. Nature 334(6183), 585-91. (hereinafter “Haseloff & Gerlach”).

Hemmings-Mieszczak, M., Steger, G. & Hohn, T. J. (1997). J. Mol. Biol. 267, 1075-88.

Hendry, P., McCall, M. J., Lockett, T. J. (1997) Design of hybridizing arms in hammerhead ribozymes. Methods Mol. Biol. 74, 253-264.

Hertel, K. J., Herschlag, D., Ulhlenbeck, O. C. (1996) Specificity of hammerhead ribozyme cleavage. EMBO J. 15, 3751-7.

Higgs, P. G. (1995) Thermodynamic properties of transfer RNA: a computational study. J. Chem. Soc. Faraday Trans 91(16), 25431-2540. (hereinafter “Higgs”)

Ho, S. P., Bao, Y., Lesher, T., Malhotra, R., Ma, L. Y., Fluharty, S. J., Sakai, R. R. (1998) Mapping of RNA accessible sites for antisense experiments with oligonucleotide libraries. Nat. Biotechnol. 16, 59-63.

Ho, S. P., Britton, D. H., Stone, B. A., Behrens, D. L., Leffet, L. M., Hobbs, F. W., Miller, J. A., Trainor, G. L. (1996) Potent antisense oligonucleotides to the human multidrug resistance-1 mRNA are rationally selected by mapping RNA-accessible sites with oligonucleotide libraries. Nucleic Acids Res. 24, 1901-7.

Hofacker, I. L., Fontana, W., Stadler, P. F., Bonhöffer, S., Tacker, M. and Schuster, P. (1994) Fast folding and comparison of RNA secondary structures. Monatshefte f Chemie 125, 167-188.

Holen, T., Amarzguioui, M., Wiiger, M. T., Babaie, E., Prydz, H. (2002) Positional effects of short interfering RNAs targeting the human coagulation trigger Tissue Factor. Nucleic Acids Res. 30, 1757-66.

Iserentant, D. & Fiers, W. (1980). Gene 9, 1-12.

Jagadeeswaran, P. & Cherayil, J. D. (1980). J. Theor Biol. 83, 369-75.

James, W. and Cowe, E. (1997) Computational approaches to the identification of ribozyme target sites. Methods Mol. Biol. 74, 17-26.

Kashani-Sabet, M., Liu, Y., Fong, S., Desprez, P. Y., Liu, S., Tu, G., Nosrati, M., Handumrongkul, C., Liggitt, D., Thor, A. D., Debs, R. J. (2002) Identification of gene function and functional pathways by systemic plasmid-based ribozyme targeting in adult mice. Proc. Natl. Acad. Sci. USA. 99, 3878-83.

Kawasaki, H., Taira, K. A. (2002) A functional gene discovery in the Fas-mediated pathway to apoptosis by analysis of transiently expressed randomized hybrid-ribozyme libraries. Nucleic Acids Res. 30, 3609-14.

Kawasaki, H., Onuki, R., Suyama, E., Taira, K. (2002) Identification of genes that function in the TNF-alpha-mediated apoptotic pathway using randomized hybrid ribozyme libraries. Nat. Biotechnol. 20, 376-80.

Kolter, R., and Yanofsky, C. (1982). Annu. Rev. Genet. 16, 113-34.

Konings, D. A., Gutell, R. R. (1995) A comparison of thermodynamic foldings with comparatively derived structures of 16S and 16S-like rRNAs. RNA 1, 559-74.

Kore, A. R., Vaish, N. K., Kutzke, U., Eckstein, F. (1998) Sequence specificity of the hammerhead ribozyme revisited; the NHH rule. Nucleic Acids Res. 26, 4116-20.

Kowalski, P., Stein, U., Scheffer, G. L., Lage, H. (2002) Modulation of the atypical multidrug-resistant phenotype by a hammerhead ribozyme directed against the ABC transporter BCRP/MXR/ABCG2. Cancer Gene Ther. 9, 579-86.

Kowalski, P., Wichert, A., Holm, P. S., Dietel, M., and Lage, H. (2001) Selection and characterization of a high-activity ribozyme directed against the antineoplastic drug resistance-associated ABC transporter BCRP/MXR/ABCG2. Cancer Gene Ther. 8, 185-192.

Lagos-Quintana, M., Rauhut, R., Lendeckel, W., Tuschl, T. (2001) Identification of novel genes coding for small expressed RNAs. Science 294, 853-8.

Lai, E. C. (2002). Micro RNAs are complementary to 3′ UTR sequence motifs that mediate negative post-transcriptional regulation. Nature Genet. 30, 363-4.

Lander et al., International Human Genome Sequencing Consortium (IHGSC) (2001). Initial sequencing and analysis of the human genome. Nature 409, 860-921.

Landick, R., Turnbough, C. L., Yanofsky, C. (1996) in Escherichia Coli and Salmonella: Cellular and Molecular Biology, eds. Neidhardt, F. C., Curtiss, R., Lin, E. C. (American Society for Microbiology, 2nd Edition, Washington, D.C.), pp.1263-1286.

Lau, N. C., Lim, L. P., Weinstein, E. G., Bartel, D. P. (2001) An abundant class of tiny RNAs with probable regulatory roles in Caenorhabditis elegans. Science 294:858-62.

Lazinski, D. W. and Taylor J. M. (1995). RNA 1, 225-233.

Le, S. Y., Chen, J. H., Braun, M. J., Gonda, M. A. and Maizel, J. V. (1988) Stability of RNA stem-loop structure and distribution of non-random structure in the human immunodeficiency virus (HIV-I). Nucleic Acids Res. 16, 5153-5168.

LeCuyer, K. A. & Crothers, D. M. (1993). The Leptomonas collosoma spliced leader RNA can switch between two alternate structural forms. Biochemistry 32, 5301-5311.

Lee, R. C., Ambros, V. (2001) An extensive class of small RNAs in Caenorhabditis elegans. Science 294, 862-4.

Lee, N. S., Dohjima, T., Bauer, G., Li, H., Li, M. J., Ehsani, A., Salvaterra, P., Rossi, J. (2002) Expression of small interfering RNAs targeted against HIV-1 rev transcripts in human cells. Nat. Biotechnol. 20, 500-5.

Li, Q. X., Robbins, J. M., Welch, P. J., Wong-Staal, F., Barber, J. R. (2000) A novel functional genomics approach identifies mTERT as a suppressor of fibroblast transformation. Nucleic Acids Res. 28, 2605-12.

Lieber, A., Strauss, M. (1995) Selection of efficient cleavage sites in target RNAs by using a ribozyme expression library. Mol. Cell. Biol. 15, 540-51.

Lima, W. F., Monia, B. P., Ecker, D. J. and Freier, S. M. (1992) Implication of RNA structure on antisense oligonucleotide hybridization kinetics. Biochemistry 31, 12055-12061.

Lockhart, D. J., Dong, H., Byrne, M. C., Follettie, M. T., Gallo, A V., Chee, M. S., Mittmann, M., Wang, C., Kobayashi, M., Horton, H., Brown, E. L. (1996). Expression monitoring by hybridization to high-density oligonucleotide arrays. Nat. Biotechnol. 14 (13):1675-80.

LyngsØ, R. B., Zuker, M. & Pedersen, C. N. S. (1999). Bioinformatics 15, 440-445.

Marshall, E. (1999). Drug firms to create public database of genetic mutations. Science 284 (5413):406-7.

Martinez, H. M. (1984) An RNA folding rule. Nucl. Acids Res. 12, 323-334.

Martinez, H. M. (1988) An RNA secondary structure workbench. Nucleic Acids Res. 16, 1789-1798.

Mathews, D. H., Burkard, M. E., Freier, S. M., Wyatt, J. R., Turner, D. H. (1999) Predicting oligonucleotide affinity to nucleic acid targets. RNA 5, 1458-69.

Mathews, D. H., Sabina, J., Zuker, M. and Turner, D. H. (1999) Expanded Sequence Dependence of Thermodynamic Parameters Provides Robust Prediction of RNA Secondary Structure. J Mol Biol. 288, 911-940. (hereinafter “Mathews et al.”)

Matveeva, O., Felden, B., Audlin, S., Gesteland, R. F., Atkins, J. F. (1997) A rapid in vitro method for obtaining RNA accessibility patterns for complementary DNA probes: correlation with an intracellular pattern and known RNA structures. Nucleic Acids Res. 25, 5010-6.

Matveeva, O., Felden, B., Tsodikov, A., Johnston, J., Monia, B. P., Atkins, J. F., Gesteland, R. F., Freier, S. M. (1998) Prediction of antisense oligonucleotide efficacy by in vitro methods. Nat. Biotechnol. 16, 1374-5.

McCarthy, J. J., Hilfiker, R. (2000) The use of single-nucleotide polymorphism maps in pharmacogenomics. Nat. Biotechnol. 18 (5), 505-8.

Milner, N., Mir, K. U. and Southern, E. M. (1997) Selecting effective antisense reagents on combinatorial oligonucleotide arrays. Nat. Biotechnol. 15, 537-541. (hereinafter “Milner et al.”)

Mir, K. U. and Southern, E. M. (1999) Determining the influence of structure on hybridization using oligonucleotide arrays. Nat Biotechnol. 17, 788-92. (hereinafter “Mir & Southern”).

Mirmira, S. R. and Tinoco, I. Jr. (1996) NMR structure of a bacteriophage T4 RNA hairpin involved in translational repression. Biochemistry 35, 7664-74.

Mironov, A. A., Dyakonova, L. P. and Kister, A. E. (1985) A kinetic approach to the prediction of RNA secondary structures. J. Biomol. Struct. Dyn. 2, 953-962.

Mironov, A. A. and Lebedev, V. F. (1993) A kinetic model of RNA folding. Biosystems 30, 49-56.

Nowakowski, J. and Tinoco, I. Jr. (1999) RNA structure in solution. In N. Stephen (ed.) Oxford Handbook of Nuclei Acid Structures. Oxford University Press, New York, N.Y., 567-602.

Ohlstein, E. H., Ruffolo, R. R. Jr, Elliott, J. D. (2000) Drug discovery in the next millennium. Annu. Rev. Pharmacol. Toxicol. 40, 177-91.

Pan, W. H., Devlin, H. F., Kelley, C., Isom, H. C., Clawson, G. A. (2001) A selection system for identifying accessible sites in target RNAs. RNA 7, 610-21.

Pérez-Ruiz, M., Barroso-DelJesus, A., Berzal-Herranz, A. (1999) Specificity of the hairpin ribozyme. Sequence requirements surrounding the cleavage site. J Biol. Chem. 274, 29376-80.

Pierce, M. L., Ruffner, D. E. (1998) Construction of a directed hammerhead ribozyme library: towards the identification of optimal target sites for antisense-mediated gene inhibition. Nucleic Acids Res. 26, 5093-101.

Phillips, M. I., Zhang, Y. C. (2000) Basic principles of using antisense oligonucleotides in vivo. Methods Enzymol. 313, 46-56.

Quigley, G. J., Gehrke, L., Roth, D. A., Auron, P. E. (1984). Computer-aided nucleic acid secondary structure modeling incorporating enzymatic digestion data. Nucleic Acids Res. 12 (1 Pt 1), 347-66.

Rossi, J. J. (1999) Ribozymes, genomics and therapeutics. Chem. Biol. 6, R33-7.

Rossi, J. J. (1995) Controlled, targeted, intracellular expression of ribozymes: progress and problems. Trends Biotechnol. 13, 301-306.

SantaLucia J. Jr. (1998) A unified view of polymer, dumbbell, and oligonucleotide DNA nearest-neighbor thermodynamics. Proc. Natl. Acad. Sci. U.S.A. 95, 1460-5. (hereinafter “SantaLucia”).

Scherr, M., Rossi, J. J. (1998) Rapid determination and quantitation of the accessibility to native RNAs by antisense oligodeoxynucleotides in murine cell extracts. Nucleic Acids Res. 26, 5079-85.

Schuster, P, Stadler, P. F. (1994) Landscapes: complex optimization problems and biopolymer structures. Comput Chem. 18 (3): 295-324. (hereinafter “Schuster & Stadler”)

Sczakiel, G., Tabler, M. (1997) Computer-aided calculation of the local folding potential of target RNA and its use for ribozyme design. Methods Mol. Biol. 74, 11-5.

Sczakiel, G., Homann, M. and Rittner, K. (1993) Computer-aided search for effective antisense RNA target sequences of the human immunodeficiency virus type 1. Antisense Res Dev. 3,45-52.

Shippy, R., Lockner, R., Farnsworth, M., Hampel, A. (1999) The hairpin ribozyme. Discovery, mechanism, and development for gene therapy. Mol. Biotechnol. 12(1), 117-29. (hereinafter “Shippy et al.”).

Sohail, M., Southern, E. M. (2000) Selecting optimal antisense reagents. Adv. Drug. Deliv. Rev. 44 (1), 23-34.

Sohail, M., Akhtar, S., Southern, E. M. (1999) The folding of large RNAs studied by hybridization to arrays of complementary oligonucleotides. RNA 5, 646-55.

Southern, E. M., Milner, N., Mir, K. U. (1997) Discovering antisense reagents by hybridization of RNA to oligonucleotide arrays. Ciba Found. Symp. 209 38-44.

Southern, E., Mir, K., Shchepinov, M. (1999) Molecular interactions on microarrays. Nat. Genet. 21 (1 Suppl), 5-9.

Sprinzl, M:, Horn, C., Brown, M., Ioudovitch, A. and Steinberg, S. (1998) Compilation of tRNA sequences and sequences of tRNA genes. Nucleic Acids Res. 26, 148-153.

Stein, C. A. (1999) Keeping the biotechnology of antisense in context. Nat. Biotechnol. 17, 209-212.

Stein, C. A. (1999) Two problems in antisense biotechnology: in vitro delivery and the design of antisense experiments. Biochim. Biophys. Acta. 1489 (1), 45-52.

Stormo, G. (1986). In Maximizing Gene Expression, eds. Reznikoff, W. & Golg, L. (Butterworth Publishers, Stoneham, Mass.), pp. 195-224.

Stull, R. A., Taylor, L. A. and Szoka, F. C. Jr. (1992) Predicting antisense oligonucleotide inhibitory efficacy: a computational approach using histograms and thermodynamic indices. Nucleic Acids Res. 20, 3501-3508. (hereinafter “Stull et al.”)

Sugimoto, N., Nakano, S., Katoh, M., Matsumura, A., Nakamuta, H., Ohmichi, T., Yoneyama, M., Sasaki, M. (1995) Thermodynamic parameters to predict stability of RNA/DNA hybrid duplexes. Biochemistry 34 (35), 11211-6. (hereinafter “Sugimotol et al.”)

Szewczak, A. A., Cech, T. R. (1997) An RNA internal loop acts as a hinge to facilitate ribozyme folding and catalysis. RNA 3, 838-49.

Szymanski, M., Specht, T., Barciszewska, M. Z., Barciszewski, J. & Erdmann, V. A. (1998) 5S rRNA Data Bank. Nucleic Acids Res. 26, 156-159.

Tanner, N. K, (1999) Ribozymes: the characteristics and properties of catalytic RNAs. FEMS Microbiol. Rev. 23 (3), 257-75. (hereinafter “Tanner”).

Taylor, M. F., Wiederholt, K., Sverdrup, F. (1999) Antisense oligonucleotides: a systematic high-throughput approach to target validation and gene function determination. Drug Discov. Today 4, 562-567.

Thompson, J. D. (1999) Shortcuts from gene sequence to function. Nat. Biotechnol. 17, 1158-9.

Tyagi S., Kramer, F. R. (1996) Molecular beacons: probes that fluoresce upon hybridization. Nat. Biotechnol. 14, 303-8. (hereinafter “Tyagi & Kramer”).

Vanhée-Brossollet, C., Vaquero, C. (1998) Do natural antisense transcripts make sense in eukaryote? Gene 211, 1-9.

Venter, J. C. et al. (2001). The sequence of the human genome. Science 291, 1304-1351.

Vickers, T. A., Wyatt, J. R., Freier, S. M. (2000) Effects of RNA secondary structure on cellular antisense activity. Nucleic Acids Res. 28, 1340-7.

Walter, A. E., Turner, D. H., Kim, J., Lyttle, M. H., Muller, P., Mathews, D. H. and Zuker, M. (1994) Coaxial stacking of helixes enhances binding of oligoribonucleotides and improves predictions of RNA folding. Proc. Natl. Acad. Sci. 91, 9218-22.

Walton, S. P., Stephanopoulos, G. N., Yarmush, M. L., Roth, C. M. (1999) Prediction of antisense oligonucleotide binding affinity to a structured RNA target. Biotechnol. Bioeng. 65 (1), 1-9. (hereinafter “Walton et al.”).

Weidner, H., Yuan, R., Crothers, D. M. (1977) Nature 266, 193-194.

Wianny, F., Zernicka-Goetz, M. (2000) Specific interference with gene function by double-stranded RNA in early mouse development. Nature Cell Biol. 2(2), 70-5.

Williams A. L., Jr Tinoco, I. Jr. (1986) Nucleic Acids Res. 14, 299-315.

Wool, I. G., -Cluck & Endo, Y. (1992). Trends in Biochemical Sciences 17, 266-269.

Woolf, T. M., Melton, D. A., Jennings, C. G. (1992) Specificity of antisense oligonucleotides in vivo. Proc. Natl. Acad. Sci. USA 89, 7305-9.

Xia, T., SantaLucia, J. Jr, Burkard, M. E., Kierzek, R., Schroeder, S. J., Jiao, X., Cox, C., Turner, D. H. (1998) Thermodynamic parameters for an expanded nearest-neighbor model for on of RNA duplexes with Watson-Crick base pairs. Biochemistry 37 (42), 14719-35. (hereinafter “Xia et al.”)

Yu, Q., Burke, J. (1997) Design of hairpin Ribozymes for in vitro and cellular applications. Methods Mol. Biol. 74, 161-169. (hereinafter “Yu & Burke”)

Zamecnik, P. C., Stephenson, M. L. (1978) Inhibition of Rous sarcoma virus replication and cell transformation by a specific oligodeoxynucleotide. Proc. Natl. Acad. Sci.75 (1), 289-294.

Zhao, J. J. and Lemke, G. (1998) Rules for Ribozymes. Mol. Cell Neurosci. 11, 92-97.

zu Putlitz, J., Yu, Q., Burke, J. M., Wands, J. R. (1999) Combinatorial screening and intracellular antiviral activity of hairpin ribozymes directed against hepatitis B virus. J. Virol. 73, 5381-7.

Zuker, M. (2000) Calculating nucleic acid secondary structure. Curr. Opin. Struct. Biol. 10, 303-10.

Zuker, M., Jacobson, A. B. (1995) “Well-determined” regions in RNA secondary structure prediction: analysis of small subunit ribosomal RNA. Nucleic Acids Res. 23, 2791-8.

### OBJECTS AND SUMMARY OF THE INVENTION

The present invention was made in consideration of the above problem and may have as an object the provision of an efficient and statistically unbiased method of predicting structural characteristics of a nucleic acid molecule.

Another object of the invention can be to provide a method of predicting structural characteristics of an RNA molecule for identifying accessible sites for targeting by antisense nucleic acids (antisense oligos, trans-cleaving ribozymes, short interfering RNAs (siRNAs), and antisense RNAs), for predicting molecular interactions, and for design of nucleic acid probes.

Other objects and advantages of the invention may in part be obvious and may in part be apparent from the specification and the drawings.

To address the above-described problems and objects, a novel RNA folding algorithm is provided. The algorithm has been shown to offer substantial improvement for predicting single-stranded regions in RNA secondary structure. These unstructured regions are important for binding by antisense nucleic acids. Thus, use of the algorithm in methods and computer systems implementing such methods can offer an improvement in predicting single-stranded regions in RNA secondary structure; and predicting single-stranded regions in RNA secondary structure is useful in antisense, ribozyme and RNAi techniques and other applications, e.g., as discussed herein and in documents incorporated herein by reference.

In accordance with an embodiment of the invention, a computer system (say, a general purpose computer), which may include a processor, may be used for executing a number of system interface and statistical analysis instructions (e.g., software applications), which may include an embodiment of the algorithm of the present invention. The system may further include an interface for receiving sequence information (from, say, a memory device storing fragments for sampling, user input, a sequencing apparatus, and the like) and outputting structural information, programming interface for programming new models (e.g., targeting criteria) and functionality, and the like. The system may also be part of any integrated system for secondary structure and/or target accessibility prediction, antisense nucleic acid design, nucleic acid probe design, and the like.

The statistical sampling algorithm for RNA secondary structure prediction according to an embodiment of the invention generates a statistically representative sample of probable structures according to the Boltzmann probabilities of RNA secondary structures:

P(I)=(1/U)exp[−E(S,I)/RT]

where S is an RNA sequence, I is a secondary structure, E(SI) is the free energy of the structure for the sequence, R is the gas constant, T is the absolute temperature, and U is the partition function for all admissible secondary structures of an RNA sequence, i.e., U=3ₛexp[−E(S,I)/RT]. Sampling from the Boltzmann distribution is desirable, because it provides a complete statistical characterization of the ensemble of probable structures. However, because there are an exponential number of possible structures for an RNA sequence, the usual statistical sampling from a discrete probability distribution is not feasible. The solution is to employ a recursive algorithm. The algorithm in accordance with an embodiment of the invention consists of two steps: in the forward step, partition functions are computed; in the sampling step, sampling probabilities are computed and a sample of structures are generated. The improvements in structure predictions and important features previously unavailable are demonstrated below.

Probability Profiling for Prediction of Accessibility for Targeting by Antisense Nucleic Acids

For target accessibility evaluation, it is important to predict the chance that a segment of consecutive bases is single-stranded. Several unpaired bases in a row are important for the nucleation step of hybridization, which establishes stable stacking necessary for hybridization elongation. This need is addressed by extending a sampling algorithm in accordance with an embodiment of the invention for the construction of a probability profile for a target RNA molecule. There are several advantages to the profile approach to target accessibility prediction. There is a significant correlation between hybridization potential predicted by the probability profile and the degree of translation inhibition. In contrast, there is a lack of correlation with the minimum free energy structure (e.g., computed by mfold), and also a lack of correlation with previously proposed ad hoe thermodynamic indices. In designing antisense oligonucleotides using mfold, a practical problem is how to select a secondary structure for the target RNA from the optimal structure(s) and many suboptimal structures with similar free energies. By summarizing the information from a statistical sample of probable secondary structures in a single plot according to an embodiment of the invention, the probability profile not only presents a solution for this dilemma, but also reveals “well determined” single-stranded regions through the rigorous assignment of probabilities as measures of confidence in predictions.

Rational Design of RNA-Targeting Therapeutics

The probability profile generated in accordance with the invention reveals regions with high potential for hybridization between the target and an antisense nucleic acids. The identification of these regions provides useful input for the rational design of potent antisense oligos, trans-cleaving ribozymes and siRNAs as RNA-targeting therapeutics. The probability profile approach offers a comprehensive computational screening for the entire mRNA or viral RNA. For several mRNA sequences with length ranging from 1 kb to 3 kb, fifteen to twenty high hybridization sites per kb have been observed. These sites provide ample opportunities for the design and testing for potent antisense nucleic acids. This could be useful for the development of RNA-targeting therapeutics.

Functional Genomics and Drug Target Validation

The completion of the sequencing of the human genome signals the dawn of a new era in biomedical research. Of the estimated 30,000 ! 40,000 genes in the human genome, definitive functions have been assigned to only a few percent. Functional genomics is concerned with the determination of biological functions for all of the genes and their protein products on a genome-wide scale. Inactivation of a gene is the classical approach to assign a function to a gene in higher organisms. In the post-genomic era, however, gene knockout and mutagenesis, the traditional “gold standard” tools, can no longer keep pace with new sequence information rapidly accumulated from various genome projects. Therefore, antisense nucleic acids that target mRNA have emerged as attractive reverse genetic tools for high throughput functional genomics.

Thousands of new potential therapeutic targets have emerged from human genome sequencing. The selection and validation of molecular targets may be very useful for drug development in the new millennium. Antisense nucleic acids are useful tools for the validation of human therapeutic targets by means of gene modulation.

High Throughout Applications

DNA expression arrays have emerged as major high-throughput experimental tools in the post-genomic era. DNA expression arrays can provide important clues to gene function through statistical clustering analysis. Gene expression data tend to organize genes into functional categories. Genes with unknown function can be assigned tentative functions or a role in a biological process based on the known function of genes in the same cluster. Single-nucleotide polymorphism (“SNP”) databases enable studies of the association between a SNP and the risk of a disease or drug response. These associations are valuable for the identification of candidate genes for disease phenotypes.

The eventual determination of the functions of the candidate genes and confirmation of gene functional predictions based on analysis of DNA expression arrays will require experimental analysis in a systematic and high throughput fashion to keep pace with the fast-growing genome, expression array and SNP databases. Antisense nucleic acids are well suited for this endeavor. Expression array and SNP databases can provide the basis for high throughput applications to functional genomics and drug target validation.

The invention accordingly comprises the several steps and the relation of one or more of such steps with respect to each of the others, and the apparatus embodying features of construction, combination(s) of elements and arrangement of parts that are adapted to effect such steps, all as exemplified in the following detailed disclosure, and the scope of the invention may be indicated in the claims.

It is noted that in this disclosure, terms such as “comprises”, “comprised”, “comprising” and the like can have the meaning attributed to it in U.S. Patent law; e.g., they can mean “includes”, “included”, “including” and the like.

These and other embodiments are disclosed or are obvious from and encompassed by, the following Detailed Description.

### BRIEF DESCRIPTION OF THE DRAWINGS

The following Detailed Description, given by way of example, but not intended to limit the invention to specific embodiments described, may be understood in conjunction with the accompanying Figures, incorporated herein by reference, in which:

FIG. 1 is a diagram illustrating a system configuration 100 in accordance with an embodiment of the invention.

FIG. 2 is a diagram illustrating types of structural elements in RNA secondary structure: helix, hairpin loop, bulge loop, interior (internal) loop and multi-branched loop.

FIG. 3 is a secondary structural diagram for the minimum free energy structure of Xlo 5S rRNA and types of structural elements: helix (formed by stacked base pairs), bulge (B loop), interior loop (I loop), hairpin loop (H loop), and multi-branched loop (M loop).

FIG. 4 is a diagram illustrating mutually exclusive cases in the derivation of recursion for u(i,j) (equation (4)), by considering a fragment Rᵢⱼ being single stranded or the base pair rₕ-rₗ closest to the 5′ end of the fragment (i.e., the first (h-i) bases are single stranded): (a) Rᵢⱼ is single stranded; (b) h=i, l=j; (c) i<h<l=j; (d) h=i<l<j; (e) i<h<l<j.

FIG. 5 is a flow chart diagram illustrating an algorithm for sampling an RNA secondary structure in accordance with an embodiment of the invention, where for (i,j,I) for the fragment from an ith to a jth base, I=1 if it is known the ends form a pair, and I=0 if this pair is unknown.

FIG. 6 is table (Table 1) demonstrating that the algorithm samples secondary structures exactly and rigorously from the Boltzmann equilibrium probability distribution (equation (1)).

FIG. 7 is a table (Table 2) demonstrating the fast sampling step of the algorithm.

FIGS. 8A, 8B, and 8C are two-dimensional histograms (2Dhist) for classes 1A, 1B and 1C for L. collosoma SL RNA. The 2Dhist shows the frequencies of base pairs on the upper left triangle with nucleotide position on both axes. Within each histogram, the sizes of the solid squares are proportional to the frequencies of the base pairs.

FIGS. 9A and 9B are two-dimensional histograms for the classes 2A and 2B for L. collosoma SL RNA.

FIGS. 10A, 10B and 10C are diagrams illustrating the representative structures for classes 1A, 1B and 1C for L. collosoma SL RNA based on an algorithm in accordance with an embodiment of the present invention, where FIG. 10A shows structure form 1 for class 1A, FIG. 10B shows the optimal folding by mfold for class1B, and FIG. 10C shows the representative for class 1C.

FIGS. 11A and 11B are diagrams illustrating the representative structures for classes 2A and 2B for L. collosoma SL RNA based on an algorithm in accordance with an embodiment of the present invention, where FIG. 11A shows structure form 2 for class 2A and FIG. 11B shows the representative for class 2B.

FIG. 12 is a table (Table 3) for ) listing the classification, representation, and statistical characterization of the probable secondary structures of the Boltzmann ensemble for L. collosoma SL RNA by the examination of a statistical sample of 1,000 secondary structures based on an algorithm in accordance with an embodiment of the present invention.

FIG. 13 is a bar plot for comparing the probability (estimated by the frequency in a sample) of a class (white boxed bar) with the Boltzmann probability (black bar) for the representative structure of a class. Classes are from the structure classification for L. collosoma SL RNA.

FIGS. 14A, 14B and 14C are diagrams of alternative structures for cIII mRNA. The initiation codon and the Shine-Dalgarno sequence are A⁰UG² and UΓ¹³AAGGAGΓ⁷. The substructure from the 5′ end to nucleotide A(!9) is the same for structure A and structure B, where FIG. 14A shows experimental structure A, FIG. 14B shows experimental structure B, and FIG. 14C shows structure C representing a modification of B by an additional short helix involving a part of the Shine-Dalgarno sequence.

FIG. 15 is a table (Table 4) listing probability estimates of structural motifs for cIII mRNA from a sample of 100 structures based on an algorithm in accordance with an embodiment of the present invention.

FIGS. 16A, 16B and 16C are diagrams illustrating the free energy distributions of sampled structures for L. collosoma SL RNA, where FIG. 16A illustrates the Boltzmann-probability-weighted density of states (BPWDOS), FIG. 16B displays the distribution for the probability that the free energy of a structure is within a threshold of global minimum, and FIG. 16C displays the distribution for the probability that the free energy of a structure is within an energy interval.

FIGS. 17A, 17B and 17C are diagrams illustrating the free energy distributions of sampled structures for E. coli RNase P (377 nt), where FIG. 17A illustrates the Boltzmann-probability-weighted density of states (BPWDOS), FIG. 17B displays the distribution for the probability that the free energy of a structure is within a threshold of global minimum, and FIG. 17C displays the distribution for the probability that the free energy of a structure is in an energy interval.

FIGS. 18A and 18B are diagrams illustrating probability profiles for Escherichia coli (“E. coli”) tRNA^{Ala}, with sampling estimates computed from 1,000 sampled secondary structures based on an algorithm in accordance with an embodiment of the invention, where FIG. 18A shows the probability profiles for single-stranded nucleotides (segment width W=1) indicated by the phylogenetic structure (large dots) and by the minimum free energy structure (vertical bars), estimated by the sampling algorithm (short dashed line), and computed by the Vienna RNA package (long dashed line) (For the region between C⁵ and C²⁵, the sampling estimate predicts the phylogenetic structure substantially better than the Vienna RNA package), and FIG. 18B shows the probability profiles for single-stranded sequences of four consecutive nucleotides (segment width W=4) in E. coli tRNA^{Ala} indicated by the phylogenetic structure (large dots) and by the minimum free energy structure (vertical bars), and estimated by the sampling algorithm (dashed line). (The probability profile cannot be computed by the Vienna RNA package or other existing algorithms.)

FIGS. 19A, 19B, 19C, 19D are diagrams illustrating probability profiles (segment width W=4) for other representative RNA sequences, with sampling estimates computed from 1,000 sampled secondary structures based on an algorithm in accordance with an embodiment of the invention, where FIG. 19A presents the profile indicated by the phylogenetic structure (the large dots), the sampling estimate (the dashed line), and the minimum free energy structure (vertical bars) for Xenopus laevis oocyte 5S rRNA, FIG. 19B presents the profile for E. coli 16S rRNA domain II, FIG. 19C presents the profile for E. coli RNase P, and FIG. 19D presents the profile for Group I intron from 26S rRNA of Tetrahymena thermophila. The small solid squares (adjacent squares appear to form line segments) present the profile indicated by phylogenetic structure, the dashed line is the sampling estimate, and the vertical bars represent the minimum free energy structure. For the Tetrahymena Group I intron, a six base pair double-stranded region called P3 in the phylogenetic structure is not considered here because of the creation of a pseudoknot. The current sampling algorithm may be extended to predict certain types of pseudoknots.

FIG. 20 is a table (Table 5) showing a correspondence between phylogenetically determined single-stranded regions and peaks on the probability profile based on an algorithm in accordance with an embodiment of the present invention and improvement in predictions over minimum free energy structure.

FIGS. 21A and 21B are diagrams illustrating contrasting predictions by probability profile (W=4) and mfold MFE structure for nt 1!60 region and nt 1262!1322 region of the mRNA for Homo sapiens gamma-glutamyl hydrolase (GenBank Accession No. U55206, with 66 additional nucleotides at the 5′ end).

FIG. 22 is a table (Table 6) showing a comparison of inhibition of rabbit β-globin synthesis in cell-free translation systems and hybridization potential predicted by probability profile for rabbit β-globin mRNA based on an algorithm in accordance with an embodiment of the present invention.

FIG. 23 is a table (Table 7) showing a comparison of the intensity of ASO:mRNA hybridization on the oligodeoxynucleotide array and the probability profile for the first 122 bases of rabbit β-globin mRNA based on an algorithm in accordance with an embodiment of the present invention.

FIG. 24 is a diagram illustrating the probability profile for single-stranded sequences of four consecutive nucleotides (segment width W=4) estimated by 1,000 sampled secondary structures (dashed line) based on an algorithm in accordance with an embodiment of the present invention and the profile indicated by the minimum free energy structure (vertical bars) for rabbit β-globin mRNA and the experimentally measured inhibition of antisense oligomer (ASOs) in cell-free translation systems. (The profile is shown for the region of the first 230 nucleotides that is targeted by the ASOs. The length and binding sites of the ASOs are indicated by horizontal lines with the names of the ASOs centered above or below the lines. These lines also indicate the inhibition of translation through their position on the vertical axis. The vertical axis also shows the probability for the profile with inhibition corresponding to probability multiplied by 100%).

FIG. 25 is a diagram illustrating the complete probability profile for single-stranded segments of four consecutive nucleotides (segment width=4) estimated by 1,000 sampled secondary structures for E. coli lacZ mRNA.

FIG. 26 is a diagram illustrating the nt 2200-2400 portion of the probability profile for E. coli lacZ mRNA.

FIG. 27 is a table (Table 8) listing ten antisense oligos rationally designed by probability profiling and calculation of binding energies.

FIG. 28 is a diagram illustrating the concept of mutual accessibility for RNA:RNA interactions. The seven A bases are accessible in RNA 1, and their complementary bases, the seven Us are also accessible in RNA 2.

FIGS. 29A and 29B are diagrams illustrating a graphical method for the assessment of mutual accessibility between a target RNA and an antisense RNA or a ribozyme. For a 60-nt antisense RNA (embedded in a long RNA through an expression vector) and the targeted mRNA of Homo sapiens gamma-glutamyl hydrolase, FIG. 29A shows good mutual accessibility through the overlapping high probability region between nt 730 and nt 750 on the overlaid probability profiles (segment width W=4) at the target site. For the mRNA of the Breast Cancer resistance Protein (BCRP) and the binding arms of a hammerhead ribozyme designed for a GUC cleavage sequence on the target, FIG. 29B shows fairly good mutual accessibility through the overlapping high probability segments formed by nucleotide 1889, 1890, 1891 and 1892 for the 3′ binding arm and by nucleotide 1905, 1906, 1907 and 1908 for the 5′ binding arm, respectively (segment width W=1 for the overlaid probability profiles). Mutual accessibility for a segment of at least four consecutive bases may be necessary for antisense nucleation.

FIGS. 30A and 30B are diagrams illustrating models of secondary structures and sequence requirements for the hammerhead ribozyme with targeting sequence NUH9 and the hairpin ribozyme with targeting sequence BN9GUC, where N=A, C, G or U; H=A,C or U; N′ or H′ are complementary nucleotide of N or H′; Y is a pyrimidine nucleotide (U or C); R is a purine (A or G); B=C, U or G; V=G, A or C; bold letters indicate invariant nucleotides; arrow indicates the site if cleavage.

FIG. 31 is a diagram illustrating the probability profile for exon 3 (nt 1003-1119) of human estrogen receptor 1 (ESR1) mRNA (6450 nt, GenBank Accession No. NM_{—}000125).

FIG. 32 is a table (Table 9) of siRNAs rationally designed with probability profiling to target AA(N19) motifs in exon 3 of the human estrogen receptor 1 (ESR1) mRNA.

FIG. 33 is a diagram illustrating a high throughput framework for functional genomics, drug target validation, and elucidation of genetic pathways. Systematic statistical analysis of DNA expression arrays and SNP databases can provide the basis for high throughput functional analysis. Integration of computational design of antisense nucleic acids and experimental techniques (e.g., oligonucleotide array) presents a rational, efficient and high throughput platform.

### DETAILED DESCRIPTION OF THE PREFERRED EMBODIMENT

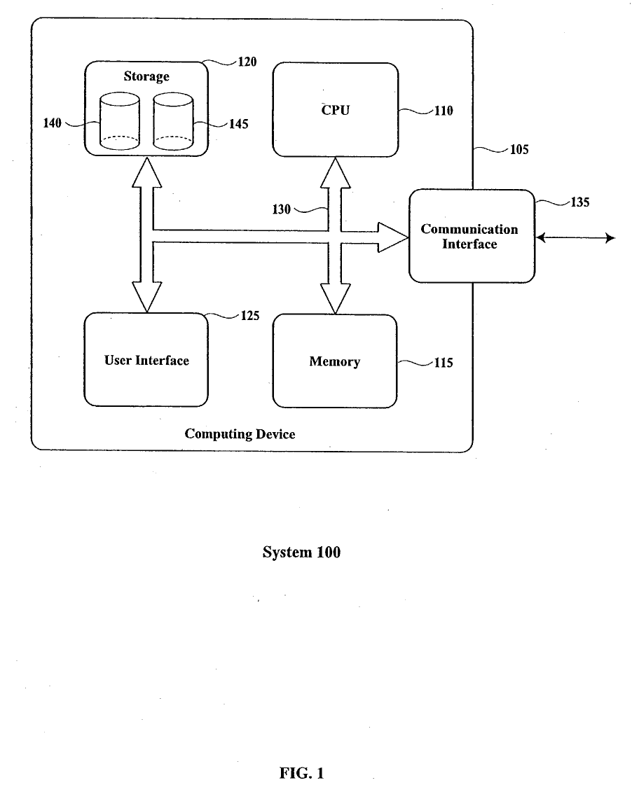

FIG. 1 is a diagram illustrating a system configuration 100 in accordance with an embodiment of the invention. As shown in FIG. 1, system 100 may comprise a computing device 105, which may be a general purpose computer (such as a PC), workstation, mainframe computer system, and so forth. Computing device 105 may include a processor device (or central processing unit “CPU”) 110, a memory device 115, a storage device 120, a user interface 125, a system bus 130, and a communication interface 135. CPU 110 may be any type of processing device for carrying out instructions, processing data, and so forth. Memory device 115 may be any type of memory device including any one or more of random access memory (“RAM”), read-only memory (“ROM”), Flash memory, Electrically Erasable Programmable Read Only Memory (“EEPROM”), and so forth. Storage device 120 may be any data storage device for reading/writing from/to any removable and/or integrated optical, magnetic, and/or optical-magneto storage medium, and the like (e.g., a hard disk, a compact disc-read-only memory “CD-ROM”, CD-ReWritable “CD-RW”, Digital Versatile Disc-ROM “DVD-ROM”, DVD-RW, and so forth). Storage device 120 may also include a controller/interface (not shown) for connecting to system bus 130. Thus, memory device 115 and storage device 120 are suitable for storing data as well as instructions for programmed processes for execution on CPU 110. User interface 125 may include a touch screen, control panel, keyboard, keypad, display or any other type of interface, which may be connected to system bus 130 through a corresponding input/output device interface/adapter (not shown). Communication interface 135 may be adapted to communicate with any type of external device, system or network (not shown), such as one or more computing devices on a local area network (“LAN”), wide area network (“WAN”), the internet, and so forth. Interface 135 may be connected directly to system bus 130, or may be connected through a suitable interface (not shown).

While the above exemplary system 100 is illustrative of the basic components of a system suitable for use with the present invention, the architecture shown should not be considered limiting since many variations of the hardware configuration are possible without departing from the present invention. As described above, system 100 provides for executing processes, by itself and/or in cooperation with one or more additional devices, that may include statistical algorithms for prediction of secondary structure of nucleic acids and for prediction of accessible target sites and rational design of antisense oligos, trans-cleaving ribozymes, and siRNAs for human therapeutics and functional genomics and drug target validation and nucleic acid probe design in accordance with the present invention. System 100 may be programmed or instructed to perform these processes according to any communication protocol, programming language on any platform. Thus, the processes may be embodied in data as well as instructions stored in memory device 115 and/or storage device 120 or received at interface 135 and/or user interface 125 for execution on CPU 110. Exemplary processes for carrying out the invention will now be described in detail.

Single-stranded regions in ribonucleic acid (“RNA”) secondary structure are important for RNA:DNA, RNA:RNA and RNA:protein interactions. In accordance with an embodiment of the invention, a probability profile approach may be used for the prediction of these regions based on a statistical algorithm for sampling RNA secondary structures. For the prediction of phylogenetically determined single-stranded regions in secondary structures of representative RNA sequences, the probability profile offers substantial improvement over the minimum free energy structure. In designing antisense nucleic acids, a practical problem is how to select a secondary structure for the target RNA from the optimal structure(s) and many suboptimal structures with similar free energies. By summarizing the information from a statistical sample of probable secondary structures in a single plot, the probability profile not only presents a solution to this dilemma, but also reveals “well-determined” single-stranded regions through the rigorous assignment of probabilities as measures of confidence in predictions. In antisense application to the rabbit (β-globin mRNA, a significant correlation between hybridization potential predicted by the probability profile and the degree of inhibition of in vitro translation suggests that the probability profile approach is valuable for the identification of accessible target sites. Coupling computational design and experimental techniques (e.g., oligonucleotide array) provides a rational, efficient framework for antisense nucleic acid screening. This framework may be used for high throughput applications to functional genomics and drug target validation.

In accordance with an embodiment of the present invention, the RNA folding problem may be formulated in a statistical framework, and the partition function method may be extended for generating a statistically representative sample of the probable structures.

In accordance with an embodiment of the invention, a sampling approach for the prediction of single-stranded regions in an RNA molecule may be used. While the structural profile provided by the inventive approach is useful for the important antisense nucleic acid applications, single-stranded regions, particularly destabilizing loops, can play many important functional roles. These include, e.g., protein binding, ribozyme binding and catalysis, binding by siRNAs and antisense RNAs, regulation of cellular processes, pseudoknot formation and tertiary interactions for kissing hairpins, bulge-loop complexes, hairpin loop-internal loop complexes, and so forth. For these applications, computational prediction of single-stranded regions can also be helpful for the experimental design for structure probing by ribonucleases (“RNases”) or chemical means.

A regulatory mechanism has been recognized where an oligonucleotide can bind to a messenger RNA through complementary base pairing to block its translation. As antiviral agents, antisense oligonucleotides can inhibit replication of RNA viruses. The discovery that oligonucleotide can play a regulatory role in gene expression led to the development of the antisense strategy to artificially control gene expression. Although variable degrees of success have been achieved in the application of antisense methods to the research of biological phenomena and human disease treatment, it has been proven that antisense oligonucleotides are able to modulate gene expression in both prokaryotes and eukaryotes.

For antisense oligonucleotides to be effective, the complementary target sequence on mRNA or viral RNA must be available for hybridization. RNA nucleotides can be inaccessible when they are sequestered in secondary structure. The usually weaker tertiary interactions and RNA-protein interactions can also be factors that affect accessibility. The identification of regions likely to remain single-stranded in RNA secondary structure is an important part of antisense technology.

Target RNA structures play a significant role in determining antisense oligonucleotide efficacy in vivo. Discovery of active antisense oligonucleotides requires identification of unstructured of the target in the cellular environment. The tightest binding of antisense oligonucleotides occurs at target sites for which disruption of the target structure is minimal, and single-stranded regions should be selected over double stranded regions in the consideration of target sites. There is a correlation between single-stranded specific probes and accessible sites for antisense targeting, but there are a few exceptions, probably due to steric hindrance that limits RNase H access. It has been speculated that duplex formation is initiated at an accessible substructure that includes a site for nucleation with unpaired bases and then propagates from the nucleation site through a “zippering” process. A hairpin of four unpaired bases can be involved in hybrid formation.

A few secondary-structure-prediction-based computational approaches to the evaluation of potential antisense targets have been reported. Thermodynamic indices may be generated by averaging relevant free energies of secondary structures generated from a Monte Carlo RNA folding algorithm based on an evolutionary heuristic. Because this Monte Carlo algorithm does not guarantee the generation of a valid statistical sample of low energy structures, the most likely structure identified using this algorithm may not necessarily be the lowest free energy structure.

For the genomic RNA (about 9700 nt) and the complementary RNA strand of the human immunodeficiency virus type 1 (“HIV-1”), local folding potential can shed light on effective antisense targets. The local folding potential may be computed for each of successive overlapping segments of a chosen window width (ranging from 50 to 400 nt) along the RNA chain, by folding each segment with mfold and computing its minimum free energy. This method may be used for assessing stable structures in HIV-1. Because long distance interactions and short term interactions between the nucleotides near the ends of the segment and the neighboring nucleotides outside the segment are ignored, this method appears to be reasonable only for relatively long window widths, as it cannot address the hybridization potential of individual nucleotides or short sequences.

The use of only the optimal folding or limited suboptimal foldings from mfold for antisense prediction is an inherent limitation of the method by Walton et al. The repeated folding for folding domains introduces additional uncertainty in predictions. Global disruption of the target structure by antisense oligos is proposed. However, an array study suggests that a duplex can only form when hybridation elongation requires little perturbation of the existing target structure (Mir & Southern). This suggests antisense hybridation only disrupts local structure of the target. Furthermore, substantial human curation appears to be necessary for this method.

A comparative analysis using mfold on twenty-two RNAs has been performed. The RNAs were previously studied for selective gene inactivation by antisense oligonucleotides and ribozymes, small catalytical RNA molecules that specifically bind to target RNAs by complementary base pairing (i.e., antisense mechanism) then cleave the target at specific sites. Despite limited representation of alternative structures by four or five suboptimal foldings, the analysis found a correlation between the predicted base-pairing accessibility of the targets and the experimental efficacy of the antisense reagents. Thus, it has been recommended that the cleavage site for ribozymes should fall within a loop of at least four nucleotides, and one, preferably both, of the 5′ and 3′ ends of the antisense segment should fall within a single-stranded rather than a stem region. Despite the inherent difficulty in selecting a representative sample of the suboptimal foldings, addressing the hybridization potential using suboptimal foldings from mfold and showing the procedure works well for the rat O×40 mRNA has been proposed.

These findings lend additional support to the importance of exploring secondary structure in the selection of antisense targets. In accordance with an embodiment of the invention, it is desirable to focus on single-stranded regions in RNA secondary structure, in particular those of at least four consecutive unpaired bases. The Vienna package can calculate the probability of a single base being unpaired, however it cannot address the hybridization potential of a region. This is not a problem for the sampling-based probability profile approach utilized in accordance with the invention, which can overcome limitations of existing computational approaches. An illustrative embodiment of the inventive approach will now be described as applied to representative RNA sequences and an antisense application to rabbit β-globin mRNA.

The Nobel Prize-winning discovery of RNA catalysis led to the development of ribozyme technology for gene inhibition. Ribozymes are catalytic RNAs that possess the dual properties of sequence-specific RNA recognition and site-specific cleavage. In other words, they first bind to the RNA target by complementary base pairing, and then cleave the target at a specific site. Among ribozymes discovered to date, the hammerhead ribozyme and the hairpin ribozyme have been of greatest interest, due to a number of significant attributes of these small ribozymes. These attributes include site-specific cleavage, multiple turnover and the ability to be exogenously delivered or endogenously expressed from a transcription cassette. In addition to increased stability, ribozymes may have other potential advantages over antisense oligos: (1) the inhibitory effect of ribozymes may include a contribution from the antisense binding step; (2) ribozyme binding to the target is more stringent; and (3) their specificity is higher due to their dual properties of sequence-specific binding and site-specific target cleavage. The trans-cleavage ability makes hammerhead and hairpin ribozymes important tools in the elucidation of the function of new genes predicted from genome sequencing projects, and in the development of antiviral agents for therapeutic applications, and in the validation of drug targets.

For antisense oligos and trans-cleaving ribozymes, it is well understood that the accessibility of the target site is among the most important factors for their intracellular efficacies. There is compelling experimental and computational evidence that, to a large extent, the accessibility of the target to antisense oligos or ribozymes is constrained by the secondary structure of the target RNA. For ribozyme design, several computational methods make accessibility predictions based on mfold. However, these methods cannot escape the limitations inherent in mfold.

In addition to antisense oligos and ribozymes, RNA interference (RNAi) by double-stranded RNAs has emerged as a powerful reverse genetic tool to silence gene expression in a wide range of eukaryotic organisms including plants, Caenorhabditis elegans, Drosophila, and mice, etc. The discovery that short double-stranded siRNAs can mediate RNAi in mammalian cells has further expanded the utility of RNAi into mammalian systems. There is experimental evidence that the potency of siRNAs is also determined by target accessibility.

Statistical Sampling of RNA Secondary Structures

A structure sampling algorithm based on free energies for stacking in helices may be used to yield a representative statistical sample of secondary structures, as described in Ding. In accordance with an embodiment of the invention, the sampling probabilities may be computed using partition functions calculated in the forward step of the algorithm. For more sophisticated and realistic energy rules, an extended algorithm may be used according to an embodiment of the invention. The forward step of this algorithm may include a recursive algorithm for partition functions. This recursive algorithm may include free energies for dangling ends and other recent free energy parameters. The backward step may take the form of a sampling algorithm; the sampling probabilities may be computed using the partition functions computed in the forward step.

The extended algorithm may accommodate up-to-date free energy rules and parameters. These include free energies for stacking in a helix, stacking for a terminal mismatch in a hairpin loop (size ≦4) or an interior loop, and penalties for hairpin, bulge, interior, and multi-branched loops. Free energies for dangling ends may be used for exterior and multi-branched loops. For hairpins, a bonus for UU and GA first mismatches (included in the terminal stacking data) and a bonus for G·U closure preceded by two G nucleotides in base pairs may be applied, and a penalty for oligo-C loops (all unpaired nucleotides are C) may be used.

The Boltzmann distribution in statistical mechanics gives the probability of a secondary structure I for an RNA sequence S at equilibrium as

P(I)=(1/U)exp[−E(S,I)/RT] (1)

where E(S,I) is the free energy of the structure, R is the gas constant, T is the absolute temperature, and U is the partition function for all admissible secondary structures of the RNA sequence, i.e., U=3ₛexp[−E(S,I)/RT]. The extended algorithm samples exactly and rigorously according to the Boltzmann distribution (1), i.e., it can generate a statistical sample of any desired size from the Boltzmann ensemble of secondary structures. The sampling process is similar to the traceback algorithm employed the dynamic programming algorithms but differs in that the base pairing is randomly sampled from Boltzmann probabilities rather than chosen to yield a minimum free energy structure. Because the probability of a structure decreases exponentially with increasing free energy, the structure with the highest frequency in the sample is most likely the minimum free energy structure. When long interior loops (e.g., size>30 nt) are disallowed, the forward step of the algorithm is cubic. The sampling step of the algorithm is stochastically quadratic in the worst case, thus it can quickly generate a large number of secondary structures.

Probability Profiling for Predicting Single-Stranded Bases and Segments

From recursively derived partition functions for an RNA sequence of n bases, recursions may be used for computing marginal base pairing probability may be Pᵢⱼ=Prob(base i and base j form a pair), then the probability that base i is unbound, i.e., single-stranded, is qᵢ=1−Σ_{(i+1)≦j≦n}Pᵢⱼ−Σ_{1≦j≦i}Pⱼᵢ. The base pair binding probabilities are not locally determined by the RNA sequence, rather, they reflect a sum over all equilibrium weighted structures in which the chosen base pair occurs. Therefore, {qᵢ} statistically describe the antisense hybridization potential for every nucleotide in the sequence. Alternatively, the sampling method presents a means to estimate qᵢ with the sampling frequency for the unbound base i. This avoids the cubic algorithm required to compute the probabilities analytically. A probability profile is then displayed by plotting {qᵢ} against the nucleotide position.

However, probabilities {qᵢ} may not provide a suitable means to assess the potential of a segments to be single-stranded and available for hybridization. More specifically, for a fragment from base i to base j, Qᵢⱼ, the probability of the fragment being single-stranded may not simply be the product of individual probabilities {qₘ}, i≦m≦j, because independence may be invalidated by the nearest-neighbor interactions. However, a probabilistic measure of the hybridization potential of a segment can be obtained from a sample of secondary structures. Because the sample is representative of the Boltzmann ensemble of secondary structures, the fraction of the sample in which all the nucleotides in the segment are single-stranded provides an unbiased estimate of the probability of the segment being single-stranded. For all successive overlapping segments of width W, the sampling estimate for the probability that a segment is single-stranded can be plotted against the first nucleotide of the sequence for a probability profile of single-stranded segments with width W. Based on a rule of thumb of at least four unpaired bases, W may be set to equal 4 for an antisense application.

An algorithm in accordance with an embodiment of the invention will now be described in detail.

As mentioned before, a recursive algorithm is presented for the partition functions of RNA secondary structures based on recent thermodynamic parameters. A fast statistical algorithm may be used with the partition functions to generate a statistical sample from the Boltzmann ensemble of secondary structures. The algorithm presents a statistical solution to the dilemma that presentation of suboptimal foldings through a designed suboptimal selection method can be limited, and that, complete enumeration and examination of all suboptimal foldings (with free energies within a threshold of the global minimum) are difficult. By classifying sampled structures, the algorithm enables an efficient statistical delineation and representation of the Boltzmann ensemble. Alternative biological structures can be revealed by a statistical sample. The sampling algorithm may be applied to Leptomonas collosoma (“L. collosoma”) Spliced Leader (“SL”) RNA and mRNA of cIII Gene of Bacteriophage λ, two examples with experimentally demonstrated alternative structures. These structures are well predicted by the sampling algorithm, while a structure for cIII mRNA is poorly predicted by mfold as a result of its algorithmic design for the selection of suboptimal foldings. Statistical sampling provides a means to estimate the probability of any structural motif with or without constraint. Furthermore, a probability profile for any specified fragment width can be constructed for predicting single-stranded regions in RNA secondary structure. By overlaying probability profiles, a mutual accessibility plot can be displayed for predicting RNA:RNA interaction. The sampling approach offers an effective means to address both the uncertainty in structure prediction and the likelihood of potential alternative structures for long-chain RNAs. In particular, the applications show that the sampling algorithm can be well suited to structure prediction and assessment of target accessibility for mRNAs. In addition, Boltzmann-probability-weighted density of states and free energy distributions of sampled structures can be readily computed. Thus, the sampling algorithm enables important features and tools for the characterization of the Boltzmann ensemble of RNA secondary structures. It also provides new tools for RNA research, in particular, for the optimal target prediction and the rational design of antisense nucleic acids for gene down-regulation.

RNA molecules play a variety of important functional roles that include catalysis, RNA splicing, regulation of transcription, and translation. The function of an RNA molecule is determined by its structure. However, it is extremely difficult to crystallize large RNA molecules. To date, crystal structure has been determined only for a few RNA molecules. Secondary structures are highly conserved in evolution for most functional RNAs, e.g., transfer RNAs. On the other hand, RNA tertiary structural motifs involve interactions between secondary structure elements. To a large extent, RNA folding is driven by secondary structure features. For these reasons, elucidation of RNA secondary structure is an important step toward determination of RNA three-dimensional structure and function.

The characterization of the full ensemble of probable RNA secondary structures has been of great interest, because from the perspective of statistical mechanics, an RNA molecule can exist in an ensemble of structures. For example, a messenger RNA (mRNA) may exist as a population of different structures. On the other hand, multiple structures are involved in a variety of RNA regulatory functions. These include the function of 5S RNA during protein synthesis, regulation of translation initiation, and transcription attenuation in enteric bacteria.

Free energy minimization has been a popular method for RNA secondary structure prediction from a single sequence. Although free energy models for secondary structure motifs have undergone refinements for more accurate characterization of folding thermodynamics, there is still uncertainty in the experimental estimates of the parameters. The free energy computed for a structure is approximate also because the assumption of free energy additivity and the need to extrapolate to loop sequences and loop sizes in the absence of measured estimates. The ill conditioning of the RNA folding problem by free energy minimization has been well noted. Furthermore, the stability of secondary structure motifs can be affected by potential tertiary interactions that are unaccounted for in secondary structure prediction, and little is known about thermodynamic contributions of tertiary motifs. Hence, the minimum free energy structure from a folding algorithm may not be the true structure, and the true structure may be a suboptimal folding. For these reasons, it is important to fully characterize and efficiently represent the Boltzmann ensemble of RNA secondary structures. However, existing algorithms have only provided partial solutions for addressing above issues.

The mathematical algorithms by Zuker predict optimal folding and present a designed set of suboptimal foldings within any prescribed P % (0≦P≦100) of the global minimum. This is an efficient approach, however, it has its limitations. For each admissible base pair, the suboptimal algorithm generates the constrained optimal folding with this pair as the constraint. Thus it regenerates the global optimal folding if the base pair is present in the global optimal folding. For a sequence of n nucleotides, and no base pairs in the optimal folding, at most n(n−1)/2−n₀ suboptimal foldings are examined by this algorithm. This set is common for all choices of P, and those within P % of the minimum free energy are returned by the algorithm. For large P and for even moderate n, this a small subset of all the suboptimal foldings within P % of the minimum free energy because as P approaches 100 the number of all suboptimal foldings increases exponentially with n. Furthermore, if the least stable structure from this set is Q% off the global minimum, then for P<Q, no new suboptimal foldings are produced. A structure which is not the constrained optimal folding generated by any of its base pairs is in the complementary set of the “missing” suboptimal foldings, i.e., the collection of suboptimal foldings excluded by the suboptimal algorithm. For example, structures specified by removing one or more base pairs from the optimal folding fall into this set.

A recent mathematical algorithm by Wuchty et al. deals with the computation of all suboptimal foldings within any specified increment of the minimum free energy. This is a more analytical treatment than an earlier attempt. For this algorithm, the number of suboptimal foldings and CPU time show exponential behavior as the range of the energy interval increases. This is the result of exponential number of structures for an RNA sequence. For even moderate sequence length and a relatively wide energy interval, enumeration and examination of this huge set of suboptimal foldings become prohibitive.

The calculation of equilibrium partition functions and base pairing probabilities is an important advance toward the characterization of the Boltzmann ensemble of secondary structures. However, the elegant algorithm for this calculation does not generate any secondary structure.

The dilemma that the presentation of suboptimal foldings through a designed set can be limited and complete enumeration and examination of suboptimal foldings are difficult appears to be impossible to solve by a mathematical treatment. While conventional algorithms fall short of the objective of efficient and statistically unbiased representation of suboptimal foldings, statistical sampling approach may not only demonstrate the optimal folding or its close resemblance, but also efficiently summarize the suboptimal foldings and reveal potentially important alternatives.

In accordance with an embodiment of the invention, an algorithm for partition functions that are based on recent free energy parameters is provided. In addition, an algorithm based on these energy parameters and the partition functions to sample exactly and rigorously from the Boltzmann distribution is provided. Prediction of alternative structures presents a challenging test on an algorithm because there are two structures to be predicted. The capability of an algorithm according to an embodiment of the present invention for predicting alternative structures is demonstrated with applications to L. collosoma SL RNA and mRNA of cIII Gene of Bacteriophage λ, two examples with experimentally demonstrated alternative structures. The classification of probable structures for L. collosoma SL RNA and probability estimates of structural motifs for cIII mRNA are also demonstrated.

Computing Partition Functions

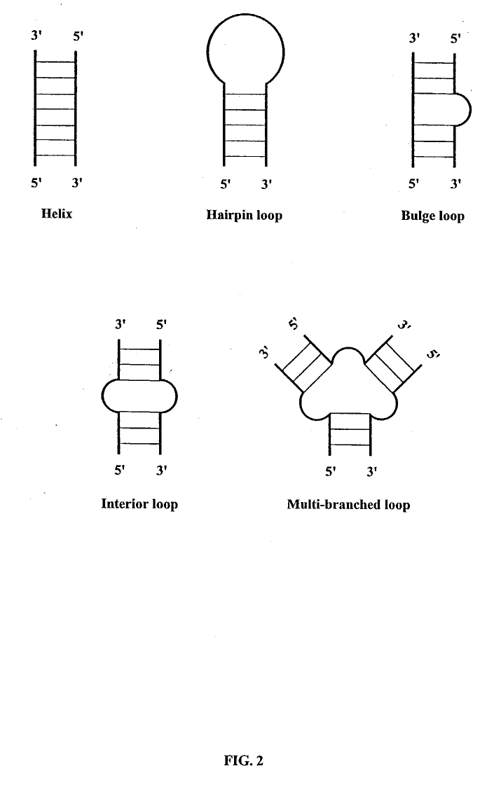

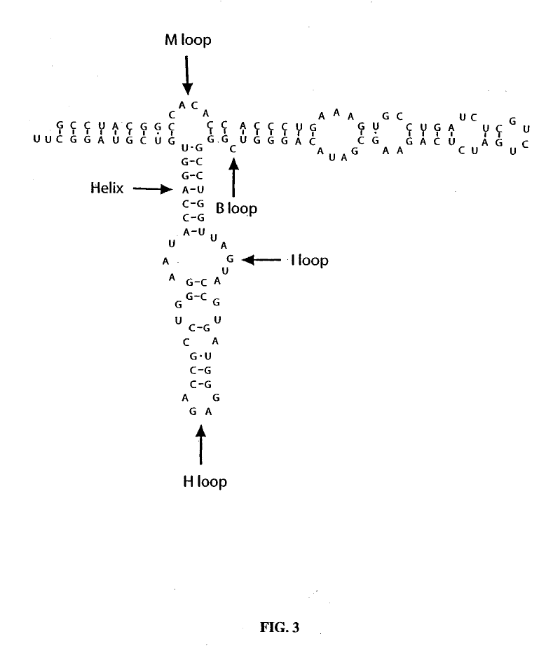

For an RNA molecule of n ribonucleotides, the sequence from the ith ribonucleotide from the 5′ end to the jth ribonucleotide may be denoted by Rᵢⱼ=rᵢrᵢ₊₁ . . . rⱼ, 1≦i,j≦n, where rᵢ=A, C, G, or U. Elements of RNA secondary structure are illustrated by FIGS. 2 and 3. Let Iᵢⱼ be a secondary structure on Rᵢⱼ that meets the usual constraints of unknotted structure and that there are at least three intervening bases between any base pair. For structures under the constraints, let IPᵢⱼ be a structure on Rᵢⱼ with the ends constrained to form a base pair. The partition functions restricted to Rᵢⱼ may be defined as:

u(i,j)=ΣIᵢⱼexp[−E(Rᵢⱼ,Iᵢⱼ)/RT] (2)

up(i,j)=ΣIPᵢⱼexp[−E(Rᵢⱼ,IPᵢⱼ)/RT] (3)

where E(Rᵢⱼ, Iᵢⱼ) is the free energy of structure Iᵢⱼ for Rᵢⱼ, R is the gas constant, T is the absolute temperature, and kcal/mol/RT=1.6225. Recursive calculation of partition functions may be used for computing base pair probabilities. The recursions presented below extend such by including all but coaxial stacking from recent free energy parameters. Also, the recursions are presented in a fashion such that sampling probabilities can be readily derived.

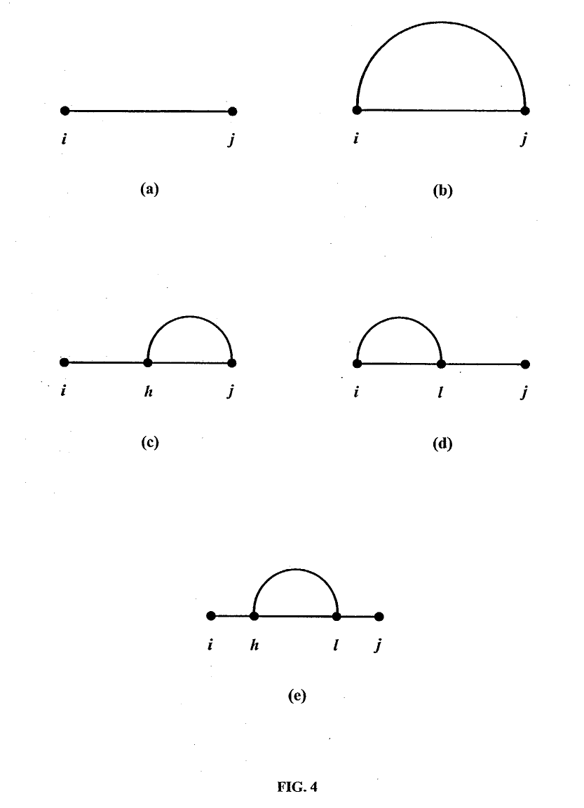

When a single stranded base is adjacent to two helices, it may be the case that only the 3′ dangling is considered because it is usually more energetically favorable than 5′ dangling according to the free energy data for dangling ends. The assumed additivity of free energy implies multiplicativity of contributions by structural elements to the partition functions. The contributions to the partition functions by mutually exclusive conformational classes are, however, additive. These features are important in the derivation of a recursive algorithm. As illustrated by FIG. 4, for fragment Rᵢⱼ, by considering it being single stranded or the base pair rₕ-rₗ closest to the 5′ end of the fragment (i.e., the first (h-i) bases are single stranded), the following mutually exclusive and exhaustive cases may be included: (a) Rᵢⱼ is single stranded; (b) h=i, l=j; (c) i<h<l=j; (d) h=i<l<j; (e) i<h<l<j. Thus, u(i,j) is a sum of five terms:

$$\begin{matrix}u ( i , j ) = 1 + up ( i , j ) exp [ - etp ( i , j ) / RT ] + \\ {\sum }_{i < h < j} up ( h , j ) exp \{ - [ ed5 ( h , j , h - 1 ) + etp ( h , j ) ] / RT \} + \\ {\sum }_{i < h < l < j} up ( h , l ) exp \{ - [ ed5 ( h , l , h - 1 ) + \\ {\sum }_{i < l < j} up ( i , l ) exp [ - etp ( i , l ) / RT ] \{ exp [ - ed3 ( i , l , l + 1 ) / RT ] u ( l + 2 , j ) + \\ u ( l + 1 , j ) - u ( l + 2 , j ) \} + \\ etp ( h , l ) ] / RT \} \{ exp [ - ed3 ( h , l , l + 1 ) / RT ] u ( l + 2 , j ) + \\ u ( l + 1 , j ) - u ( l + 2 , j ) \}\end{matrix} \qquad ( 4 )$$

where for base pair rᵢ-rⱼ, etp(i,j) is the terminal A-U, G-U penalty, and ed5(h,l, h−1) is the free energy for 5′ dangling rₕ₋₁ on rₕ-rₗ, and ed3(h,l, l+1) is the free energy for 3′ dangling rₗ₊₁ on rₕ-rₗ. When rᵢ and rⱼ form a base pair, there are the following exclusive and exhaustive cases: (i) rᵢ-rⱼ closes a hairpin; (ii) rᵢ-rⱼ is the exterior pair of a base pair stack; (iii) rᵢ-rⱼ closes a bulge or an interior loop; (iv) rᵢ-rⱼ closes a multi-branched loop. Thus up(i,j) is the sum of four contributions:

up(i,j)=exp[−eh(i,j)/RT]+exp[−es(i,j,i+1,j−1)/RT]up(i+1,j−1)+Σ_{i<h<l<j}exp[−ebi(i,j,h,l)/RT]up(h,l)+upₘ(i,j) (5)

where eh(i,j), es(i,j, i+1,j−1) and ebi(i,j,h,l) are free energies for a hairpin closed by rᵢ-rⱼ, stacking between base pairs rᵢ-rⱼ and rᵢ₊₁-rⱼ₋₁, and a bulge or an interior loop with exterior base pair rᵢ-rⱼ and interior base pair rₕ-rₗ, respectively, and upₘ(i,j) is the contribution from case (iv) above. For case (iv), by considering the internal helix closest to rᵢ with closing pair rₕ-rₗ, the recursion for upₘ(i,j) is

$$\begin{matrix}{up}_{m} ( i , j ) = {\sum }_{i + 1 < l < j} up ( i + 1 , l ) exp \{ - [ a + 2 c + \\ etp ( i + 1 , l ) ] / RT \} \{ exp [ - ed3 ( i + 1 , l , l + 1 ) / RT ] u1 ( l + 2 , j - 1 ) + \\ u1 ( l + 1 , j - 1 ) - u1 ( l + 2 , j - 1 ) \} + \\ {\sum }_{i + 2 < l < j} up ( i + 2 , l ) exp \{ - [ a + 2 c + b + ed3 ( j , i , i + 1 ) + \\ etp ( i + 2 , l ) ] / RT \} \{ exp [ - ed3 ( i + 2 , l , l + 1 ) / RT ] u1 ( l + 2 , j - 1 ) + \\ u1 ( l + 1 , j - 1 ) - u1 ( l + 2 , j - 1 ) \} + \\ {\sum }_{i + 3 \leq h < l < j} up ( h , l ) exp \{ - [ a + 2 c + ( h - i - 1 ) b + \\ ed3 ( j , i , i + 1 ) + ed5 ( h , l , h - 1 ) + \\ etp ( h , l ) ] / RT \} \{ exp [ - ed3 ( h , l , l + 1 ) / RT ] u1 ( l + 2 , j - 1 ) + \\ u1 ( l + 1 , j - 1 ) - u1 ( l + 2 , j - 1 ) \}\end{matrix} \qquad ( 6 )$$

where a, b, c are the offset, free base penalty, and helix penalty of the assumed linear penalty for a multi-branched loop: loop penalty=a+b×(number of unpaired bases)+c×(number of helices); the three sums with h=i+1, h=i+2, and h≧i+3 are for different cases of dangling on rᵢ-rⱼ and rₕ-rₗ; and u1(k,j−1) is an auxiliary partition function for a multi-branched loop with the properties that there is at least one helix between rₖ and rⱼ₋₁, and rₖ₋₁ is the 3′ end of the previous helix in the loop and rⱼ is the 3′ end base of the closing pair rᵢ-rⱼ for the loop. Similar to the derivation of upₘ(i,j), we consider the closing base pair r_{h′}-r_{l′} of the helix closest to the 5′ end of R₍ₗ₊₁₎ⱼ and take into account of both dangling energy and terminal penalty. Furthermore, we must consider both the case of no more helix between r_{l′+1} and rⱼ and the case of at least one more helix. For u1(i,j), rⱼ₊₁ is the 3′ base of the closing base pair for the multi-branched loop, and rᵢ₋₁ is the 3′ end of the previous helix in the loop. The recursion for u1(i,j) is:

$$\begin{matrix}u1 ( i , j ) = {\sum }_{i < l \leq j} up ( i , l ) exp \{ - [ c + etp ( i , l ) ] / RT \} \{ f ( j + 1 , i , l ) exp [ - ( j - l ) b / RT ] + \\ exp [ - ed3 ( i , l , l + 1 ) / RT ] u1 ( l + 2 , j ) + u1 ( l + 1 , j ) - u1 ( l + 2 , j ) \} + \\ {\sum }_{i + 1 < l \leq j} up ( i + 1 , l ) exp \{ - [ c + b + \\ etp ( i + 1 , l ) ] / RT \} \{ f ( j + 1 , i + 1 , l ) exp [ - ( j - l ) b / RT ] + \\ exp [ - ed3 ( i + 1 , l , l + 1 ) / RT ] u1 ( l + 2 , j ) + u1 ( l + 1 , j ) - u1 ( l + 2 , j ) \} \\ {\sum }_{i + 2 \leq h < l \leq j} up ( h , l ) exp \{ - [ c + ( h - i ) b + etp ( h , l ) + \\ ed5 ( h , l , h - 1 ) ] / RT \} \{ f ( j + 1 , h , l ) exp [ - ( j - l ) b / RT ] + \\ exp [ - ed3 ( h , l , l + 1 ) / RT ] u1 ( l + 2 , j ) + u1 ( l + 1 , j ) - u1 ( l + 2 , j ) \}\end{matrix} \qquad ( 7 )$$

where f(j+1,h,l)=1 for l=j and f(j+1,h,l)=exp[−ed3(h,l,l+1)/RT] for l<j. The computation is O(n⁴) for (4), (6) and (7) as written, and is O(n³) for (5) when long interior loops are disallowed. Three additional auxiliary arrays s1(h,j), s2(h,j) and s3(h,j) may also be introduced:

$$\begin{matrix}\begin{matrix}s1 ( h , j ) = {\sum }_{i < l < j} up ( h , l ) exp \{ - [ ed5 ( h , l , h - 1 ) + \\ etp ( h , l ) ] / RT \} \{ exp [ - ed3 ( h , l , l + 1 ) / RT ] u ( l + 2 , j ) + \\ u ( l + 1 , j ) - u ( l + 2 , j ) \}\end{matrix} & ( 8 ) \\ \begin{matrix}s2 ( h , j ) = {\sum }_{i < l < j} up ( h , l ) exp \{ - [ ed5 ( h , l , h - 1 ) + \\ etp ( h , l ) ] / RT \} \{ exp [ - ed3 ( h , l , l + 1 ) / RT ] u1 ( l + 2 , j - 1 ) + \\ u1 ( l + 1 , j - 1 ) - u1 ( l + 2 , j - 1 ) \}\end{matrix} & ( 9 ) \\ \begin{matrix}s3 ( h , j ) = {\sum }_{i < l \leq j} up ( h , l ) exp \{ - [ ed5 ( h , l , h - 1 ) + \\ etp ( h , l ) ] / RT \} \{ f ( j + 1 , h , l ) exp [ - ( j - l ) b / RT ] + \\ exp [ - ed3 ( h , l , l + 1 ) / RT ] u1 ( l + 2 , j ) + \\ u1 ( l + 1 , j ) - u1 ( l + 2 , j ) \}\end{matrix} & ( 10 )\end{matrix}$$

Then the quartic sum in (4) becomes Σ_{i<h<j-1}s1(h,j), the quartic sum in (6) becomes exp[(−ed3(j,i,i+1)/RT]Σ_{i+3≦h≦j-1}exp[−(a+2c+(h−i−1)b)/RT]s2(h,j), and the quartic sum in (7) becomes Σ_{i+2≦h≦j-1}exp[−(c+(h−i)b)/RT]s3(h,j). At the cost of storing these arrays, the algorithm is cubic when long interior loops (e.g., size>30) are disregarded.

The computation may be started with boundary values for short fragments and proceed to longer ones using the recursions. For 1≦i≦j≦i+3≦n, u(i,j)=1, up(i,j)=0, u1(i,j)=0, s1(i,j)=0, s2(i,j)=0, and s3(i,j)=0; for j=i+4≦n, u(i, i+4)=1+exp[−(eh(3)+etp(i,i+4))/RT], up(i, i+4)=exp[−eh(3)/RT], u1(i, i+4)=exp[−(c+eh(3)+etp(i, i+4))/RT], s1(i,i+4)=0, s2(i,i+4)=0, and s3(i, i+4)=exp[−(eh(3)+etp(i,i+4)+ed5(i, i+4, i−1)/RT]; for 1≦i≦n, u(i+1, i)=1, u1(i+1, i)=0; and for 1≦i≦n−1, u1(i+2,i)=0.

The algorithm accommodates the recent free energy rules and parameters with the exception of coaxially stacking. In particular, free energies for dangling ends are incorporated analytically and rigorously. These include free energies for stacking in a helix, stacking for a terminal mismatch in a hairpin loop (size ≧4) or an interior loop, penalties for hairpin, bulge, interior and multi-branched loops. Free energies for dangling ends are used for exterior and multibranched loops. For hairpins, a bonus for UU and GA first mismatches (included in the terminal stacking data) and a bonus for G-U closure preceded by two G nucleotides in base pairs are applied, and a penalty for oligo-C loops (all unpaired nucleotides are C) is used. A table may be consulted for tetraloops (hairpin loops with four unpaired nucleotides). For a bulge of one nucleotide, the stacking energy of the adjacent pairs may be added. For interior loops, tables for 1×2, 1×2, and 2×2 loops may be consulted and a penalty for asymmetry may be applied. A terminal A-U, G·U penalty may be explicitly applied to an exterior loop, multi-branched loops, bulges longer than one nucleotide, and triloops (hairpin loops with three unpaired nucleotides), while this penalty may be included in the terminal stacking data for hairpin loops (size ≧4 ) and interior loops. These free energy parameters are for 37° C. and 1M NaCl; however, this algorithm can be used with any set of nearest neighbor parameters derived for other conditions.

With the partition function u(1, n) available, the Boltzmann equilibrium probability for a secondary structure I₁ₙ of sequence R₁ₙ can then be computed. From a Bayesian statistics perspective, both the sequence R₁ₙ and the secondary structure I₁ₙ are random variables. Thus the Boltzmann probability in (1) can be rewritten as a conditional probability of the secondary structure given the sequence data:

P(I₁ₙ|R₁ₙ)=exp[−E(R₁ₙ, I₁ₙ)/RT]/u(1,n) (11)

Sampling Structures from the Boltzmann Distribution

Instead of presenting a minimum free energy structure, it has been shown by Ding that a statistical sample of the probable structures can be generated for a stacking-energy-based model. This task can also be accomplished for more comprehensive energy model by realizing that the recursions for partition functions correspond to sampling probabilities. For a fragment Rᵢⱼ for which it is unknown if the ends form a pair, the conditional probabilities corresponding to the five cases considered for equation (4) are given by the first five of the following six equations, respectively:

$$\begin{matrix}{P}_{0} = 1 / u ( i , j ) & & & & ( 12 ) \\ {P}_{ij} = up ( i , j ) exp [ - etp ( i , j ) / RT ] / u ( i , j ) , & & & & ( 13 ) \\ \begin{matrix}{P}_{hj} = up ( h , j ) exp \{ - [ ed5 ( h , j , h - 1 ) + \\ etp ( h , j ) ] / RT \} / u ( i , j ) ,\end{matrix} & & i < h < j & & ( 14 ) \\ \begin{matrix}{P}_{il} = up ( i , l ) exp [ - etp ( i , l ) / RT ] \{ exp [ - ed3 ( i , l , l + 1 ) / RT ] u ( l + 2 , j ) + \\ u ( l + 1 , j ) - u ( l + 2 , j ) \} / u ( i , j ) ,\end{matrix} & & i < l < j & & ( 15 ) \\ {P}_{s1h} = s1 ( h , j ) / u ( i , j ) , & & i < h < j - 1 & & ( 16 )\end{matrix}$$

Pₕₗ=up(h,l)exp{−[ed5(h,l,h−1)+etp(h,l)]/RT}{exp[−ed3(h,l,l+1)/RT]u(l+2,j)+u(l+1,j)−u(l+2,j)}/s1(h,j), h<l<j (17)

where P₀+Pᵢⱼ+Σ_{i<h<j}Pₕⱼ+Σ_{i<l<j}Pᵢₗ+Σ_{i<h<j-1}Pₛ₁ₕ=1, and Σ_{h<l<j}Pₕ₁=1. {Pₕₗ} are for sampling l after h is sampled for case (e). The computation is linear by using s1(h,j) through {Pₛ₁ₕ} and {Pₕₗ}. When the ends are known to form a base pair, the probabilities for the four cases considered for (5) are given by the first four of the following five equations, respectively:

$$\begin{matrix}{Q}_{ijH} = exp [ - eh ( i , j ) / RT ] / up ( i , j ) & & & & & & & ( 18 ) \\ {Q}_{ijS} = exp [ - es ( i , j , i + 1 , j - 1 ) / RT ] up ( i + 1 , j - 1 ) / up ( i , j ) & & & & & & & ( 19 ) \\ {Q}_{ijBI} = \{ {\sum }_{i < h < l < j} exp [ - ebi ( i , j , h , l ) / RT ] up ( h , l ) \} / up ( i , j ) & & & & & & & ( 20 ) \\ {Q}_{ijM} = {up}_{m} ( i , j ) / up ( i , j ) & & & & & & & ( 21 ) \\ \begin{matrix}{Q}_{hlBI} = exp [ - ebi ( i , j , h , l ) / RT ] up ( h , l ) / \\ {\sum }_{i < {h}^{′} < {l}^{′} < j} exp [ - ebi ( i , j , {h}^{′} , {l}^{′} ) / RT ] up ( {h}^{′} , {l}^{′} ) ,\end{matrix} & & & & & i < {h}^{′} < {l}^{′} < j & & ( 22 )\end{matrix}$$

where Q_{ijH}+Q_{ijS}+Q_{ijBI}+Q_{ijM}=1, and Σ_{i<h<l<j}Q_{hlBI}=1. For sampling h and l after the case of bulge or interior loop is sampled, {Q_{hlBI}} may need to be computed. upₘ(i,j) is the contribution to up(i,j) by the case of multi-branched loop.

In the case of a multi-branched loop, the probabilities for sampling the closing base pair rₕ₁-rₗ₁ of the first 5′ end internal helix in the loop correspond to the terms in (6) for upₘ(i,j) with the quartic term expressed in terms of s2(h,j). More specifically, we first sample h and l according to following conditional probabilities:

$$\begin{matrix}\begin{matrix}{P}_{ij ( i + 1 ) l} = up ( i + 1 , l ) exp \{ - [ a + 2 c + etp ( i + 1 , l ) ] / RT \} \times \\ \{ exp [ - ed3 \{ i + 1 , l , l + 1 ) / RT ] u1 ( l + 2 , j - 1 ) + \\ u1 ( l + 1 , j - 1 ) - u1 ( l + 2 , j - 1 ) \} / {up}_{m} ( i , j ) ,\end{matrix} & & & & & & i + 1 < l < j & & ( 23 ) \\ \begin{matrix}{P}_{ij ( i + 2 ) l} = up ( i + 2 , l ) exp \{ - [ a + 2 c + b + \\ ed3 ( j , i , i + 1 ) + etp ( i + 2 , l ) ] / RT \} \times \\ \{ exp [ - ed3 \{ i + 2 , l , l + 1 ) / RT ] u1 ( l + 2 , j - 1 ) + \\ u1 ( l + 1 , j - 1 ) - u1 ( l + 2 , j - 1 ) \} / {up}_{m} ( i , j ) ,\end{matrix} & & & & & & i + 2 < l < j & & ( 24 ) \\ \begin{matrix}{P}_{ijs2h} = exp \{ - [ a + 2 c + ( h - i - 1 ) b + \\ ed3 ( j , i , i + 1 ) ] / RT \} s2 ( h , j ) / {up}_{m} ( i , j ) ,\end{matrix} & & & & & & i + 3 \leq h < j - 1 & & ( 25 ) \\ \begin{matrix}{P}_{ijhl} = up ( h , l ) exp \{ - [ ed5 ( h , l , h - 1 ) + etp ( h , l ) ] / RT \} \times \\ \{ exp [ - ed3 \{ h , l , l + 1 ) / RT ] u1 ( l + 2 , j - 1 ) + \\ u1 ( l + 1 , j - 1 ) - u1 ( l + 2 , j - 1 ) \} / s2 ( h , j ) ,\end{matrix} & & & & & & h < l < j & & ( 26 )\end{matrix}$$

where 3_{i+1<l<j}Pᵢⱼ₍ᵢ₊₁₎ₗ+3_{i+2<l<j}Pᵢⱼ₍₁₊₂₎ₗ+3_{i+3≦h<j-1}Pᵢⱼₛ₂ₕ=1, and 3_{h<l<j}Pᵢⱼₕₗ=1. {Pᵢⱼ₍ᵢ₊₁₎ₗ} are for cases when h=i+1, {Pᵢⱼ₍ᵢ₊₂₎ₗ} are for cases when h=i+2, and {Pᵢⱼₕₗ} are for sampling l after h≧i+3 is sampled from {Pᵢⱼₛ₂ₕ}. Once both h and l are sampled, the closing base pair rₕ₁-rₗ₁ of the first helix is given by setting h1=h and l1=l.

For sampling the second internal helix, the sampling probabilities for base pair rₕ₂-rₗ₂ of the helix closest to the 5′ end of R₍ₗ₁₊₁₎₍ⱼ₋₁₎ correspond to terms in (7) for u1(l1+1,j−1) (i is substituted by l1+1, and j is substituted by j−1) with the quartic term expressed in terms of s3(h,j−1). More specifically, we first sample h and l according to conditional probabilities:

$$\begin{matrix}\begin{matrix}{Q}_{( l1 + 1 ) ( j - 1 ) ( l1 + 1 ) l} = up ( l1 + 1 , l ) exp \{ - [ c + etp ( l1 + 1 , l ) ] / \\ RT \} \{ f ( j , l1 + 1 , l ) exp [ - ( j - 1 - 1 ) b / RT ] + \\ exp [ - ed3 \{ l1 + 1 , l , l + 1 ) / RT ] u1 ( l + 2 , j - 1 ) + \\ u1 ( l + 1 , j - 1 ) - u1 ( l + 2 , j - 1 ) \} / u1 ( l1 + 1 , j - 1 ) ,\end{matrix} & l1 + 1 < l \leq j - 1 & ( 27 ) \\ \begin{matrix}{Q}_{( l1 + 1 ) ( j - 1 ) ( l1 + 2 ) l} = up ( l1 + 2 , l ) exp \{ - [ c + b + etp ( l1 + 2 , l ) ] / \\ RT \} \{ f ( j , l1 + 2 , l ) exp [ - ( j - 1 - 1 ) b / RT ] + \\ exp [ - ed3 \{ l1 + 2 , l , l + 1 ) / RT ] u1 ( l + 2 , j - 1 ) + \\ u1 ( l + 1 , j - 1 ) - u1 ( l + 2 , j - 1 ) \} / u1 ( l1 + 1 , j - 1 ) ,\end{matrix} & l1 + 2 < l \leq j - 1 & ( 28 ) \\ \begin{matrix}{Q}_{( l1 + 1 ) ( j - 1 ) s3h} = exp \{ - [ c + ( h - l1 - 1 ) b ] / RT \} s3 ( h , j - 1 ) / \\ u1 ( l1 + 1 , j - 1 ) ,\end{matrix} & l1 + 3 \leq h \leq j - 2 & ( 29 ) \\ \begin{matrix}{Q}_{( j - 1 ) hl} = up ( h , l ) exp \{ - [ ed5 ( h , l , h - 1 ) + \\ etp ( h , l ) ] / RT \} \{ f ( j , h , l ) exp [ - ( j - 1 - 1 ) b / RT ] + \\ exp [ - ed3 \{ h , l , l + 1 ) / RT ] u1 ( l + 2 , j - 1 ) + \\ u1 ( l + 1 , j - 1 ) - u1 ( l + 2 , j - 1 ) \} / s3 ( h , j - 1 ) ,\end{matrix} & h < l \leq j - 1 & ( 30 )\end{matrix}$$

where 3_{l1+1<l≦j-1}Q₍ₗ₁₊₁₎₍ⱼ₋₁₎₍ₗ₁₊₁₎ₗ+3_{l1+2<l≦j-1}Q₍ₗ₁₊₁₎₍ⱼ₋₁₎₍ₗ₁₊₂₎ₗ+3_{l1+3≦h≦j-2}Q₍ₗ₁₊ₗ₎₍ⱼ₋₁₎ₛ₃ₕ=1, and 3_{h<l≦j-1}Q₍ⱼ₋₁₎ₕₗ=1. {Q₍ₗ₁₊ₗ₎₍ⱼ₋₁₎₍ₗ₁₊₁₎ₗ} are for cases when h=l1+1, {Q₍ₗ₁₊ₗ₎₍ⱼ₋₁₎₍ₗ₁₊₂₎ₗ} are for cases when h=l1+2, and {Q₍ⱼ₋₁₎ₕₗ} are for sampling l after h≧l1+3 is sampled from {Q₍ₗ₁₊ₗ₎₍ⱼ₋₁₎ₛ₃ₕ}. Once both h and l are sampled, the closing base pair rₕ₂-rₗ₂ of the second internal helix is given by setting h2=h and l2=l. Next, one must consider two possibilities: either there is no more helix in the loop or there is at least one more helix. These two mutually exclusive cases are addressed by two additive terms in (7) for u1(l1+1,j−1). These terms give the binomial probability conditional on sampled h2 and l2 for no more helix between rₗ₂₊₁ and rⱼ₋₁:

P_{Bh2l2(j-1)}=f(j,h2,l2)exp[−(j−1−l2)b/RT]/{

f(j,h2,l2)exp[−(j−1l2)b/RT]+exp[−ed3(h2,l2,l2+1)/RT]u1(l2+2,j−1)+u1(l2+1,j−1)−u1(l2+2,j−1)} (31)

and the probability of at least one more helix is 1−P_{Bh2l2(j-1)}. If no more helix is sampled, sampling is terminated for this multi-branched loop; otherwise, the closing base pair of the next internal helix is sampled, followed by another binomial sampling. This process stops whenever no more helix is sampled. At the end of this process, for L sampled internal helices with closing base pair rₕₖ-rₗₖ, 1≦k≦L, sampling probabilities are computed with (7) for u1(l(k−1)+1,j−1) (2≦k≦L). This computation and the computation for binomial sampling with probability P_{Bhklk(j-1)} are performed (L−1) times on overlapping fragments with decreasing length. P_{Bhklk(j-1)} is given by (31) with h2 and l2 substituted by hk and lk, respectively. Similar to the probability computation for (4), the computation of the sampling probabilities with (6) and (7) are linear by using s2(h,j) and s3(h,j). When long interior loops are disregarded, the probability computation for (5) is bounded by a constant.

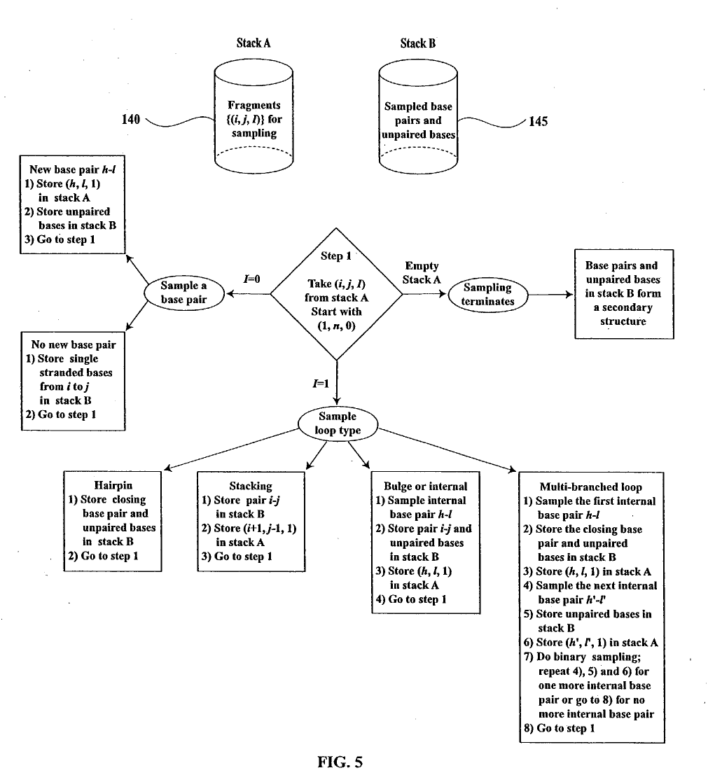

FIG. 5 is a flow diagram illustrating steps of a sampling algorithm in accordance with an embodiment of the present invention. As shown in FIG. 5, two stacks A and B are used by the sampling algorithm. In accordance with an embodiment of the invention, stacks A and B may be data stored in data storage device 120, as illustrated in FIG. 1. Stack A stores fragments {(i,j,I)} for sampling, where for the fragment from the ith base to the jth base, I=1 if it is known the ends form a pair and I=0 if this pair is unknown. Stack B collects base pairs and unpaired bases that will define a sampled secondary structure upon the completion of sampling. At the start, (1,n,0) is the only fragment in stack A. Specifically, a structure is drawn recursively as follows:

(1) Starting with R₁ₙ, draw single stranded R₁ₙ or a base pair according to probabilities P₀, Pᵢⱼ, {Pₕⱼ}, {Pᵢₗ} and {Pₛₗₕ} for i=1, j=n; if h is sampled for case (e) in the derivation for equation (4), then l is sampled with {Pₕₗ}. In case (a), i.e., single stranded R₁ₙ, the sampling is completed; in case (b), (1,n,1) is stored in stack A; in case (c), (h,n,1) is stored in A and the unpaired bases from the first base to the (h−1)th base are stored in stack B; in case (d), (1,l,1) and (l+1,n,0) are stored in stack A; in case (e), (h,l,1) and (l+1,n,0) is stored in stack A and the unpaired bases from the first base to the (h−1)th base are stored in stack B.

(2) For a new fragment Rᵢⱼ from stack A, if base pair rᵢ-rⱼ was not sampled previously, sampling for Rᵢⱼ may be performed by the same process for R₁ₙ, with 1 and n substituted by i and j, respectively.

(3) For new fragment Rᵢⱼ from stack A with ends paired (I=1), loop type may be sampled first with probabilities {Q_{ijH}}, {Q_{ijS}}, {Q_{ijBI}}, {Q_{ijM}}, and {Q_{hlBI}}; this step is followed by

(3a) For hairpin loop, the unpaired bases in the loop and the closing pair are stored in stack B as part of a sampled structure and they are no longer involved in further sampling.

(3b) For stacking, the exterior base pair (i-j) is stored in stack B and the interior base pair defines a new fragment (i+1,j−1, 1) to be stored in stack A.

(3c) For bulge or internal loop, the interior base pair in the loop (h-l) is sampled. The exterior base pair (i-j) and unpaired bases in the loop are stored in stack B and the interior base pair defines a new fragment (h, l, 1) to be stored in stack A.

(3d) For multi-branched loop, an interior base pair closest to the 5′ end of Rᵢⱼ is sampled first, a second interior base pair is then sampled. Next, one of the two cases by the Binomial distribution may need to be sampled: no more helix on the 3′ side of the loop or at least one more helix. In the latter case, another interior base pair is sampled for one more helix. For the remaining fragment on the 3′ side of the loop, the Binomial and interior base pair sampling is repeated until no more helix is sampled. Unpaired bases in the loop and rᵢ-rⱼ are stored in stack B, and new fragments defined by the interior base pairs are stored in stack A for further sampling.

During this process, after the completion of sampling for a fragment from stack A and storage of new fragment(s) in stack A and/or storage of base pair and unpaired bases in stack B, the fragment in the bottom of stack A is selected for subsequent sampling. The process terminates when stack A is empty, and a sampled secondary structure is formed by the base pairs and unpaired bases in stack B (FIG. 5).

The algorithm samples a structure exactly and rigorously from the Boltzmann equilibrium probability distribution (1) or equivalently (11), because the sampling probabilities are computed by Boltzmann conditional distribution based on partition functions restricted to fragment with or without a base pair constraint. This is obvious for the unfolded state with a free energy of 0, whose sampling probability of 1/u(1, n) is also its Boltzmann probability by (1) or (11).

From statistics mechanics perspective, there is an ensemble of probable structures and thus structure I can be viewed as a random variable. I can be expressed by an upper triangular matrix of random and dependent indicator variables Iᵢⱼ, 1≦i<j≦n. Iᵢⱼ=1 if the ith base is paired with the jth base, and Iᵢⱼ=0 otherwise. The requirement of at least three unpaired intervening bases between a base pair implies Iᵢⱼ=0 for j=i+1, i+2 and i+3, 1≦i, i+3≦n. The assumption of no pseudoknots implies IᵢⱼI_{i′j}=0 for i′<i<j′<j. Also, when base triple is prohibited, 3_{1≦i≦n}Iᵢⱼ≦1, and 3_{1≦j≦n}Iᵢⱼ≦1. Thus, I is a high dimension random variable. Sampling directly from a high dimensional probability distribution is often difficult. In some cases, however, the difficulty can be overcome by conditional sampling at lower dimension(s). More specifically, given data y, if one can sample from conditional distributions p(x₁|y), p(xₖ|x₁, . . . , xₖ₋₁,y) (k=2, . . . , m), then x=(x₁, x₂, . . . , xₖ) follows distribution p(x|y). This is the scheme adopted for secondary structure sampling. For given RNA sequence data, the new base pairs and single stranded bases are sampled by conditioning on already formed substructures from previous sampling steps. Upon the completion of the process, the collection of the substructures defines a structure sampled according to the Boltzmann equilibrium probability distribution (1) or equivalently (11).

The sampling process is similar to the traceback algorithm employed in the dynamic programming algorithms but differs in that the base pairing is randomly sampled with Boltzmann conditional probabilities rather than selected by minimum energy principle for the fragments. Because the probability of a structure decreases exponentially with increasing free energy, the most likely structure in a sample is the minimum free energy structure. In other words, the minimum free energy structure has the largest sampling probability because its Boltzmann probability is larger than any other structure.

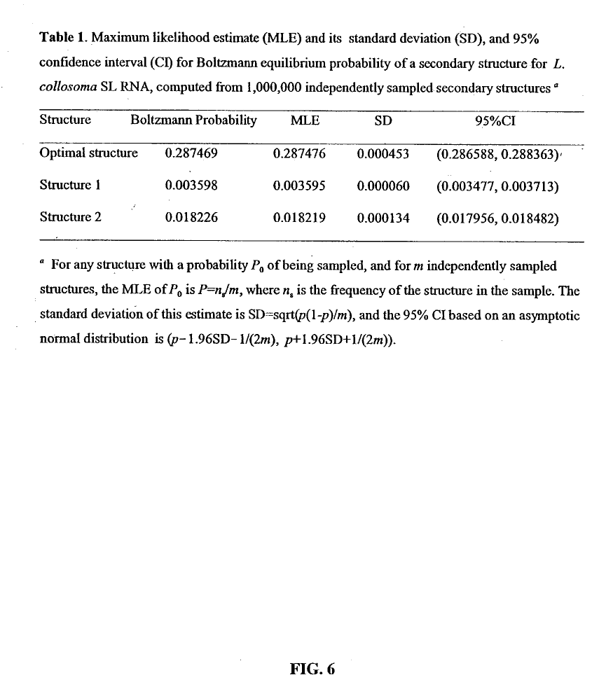

For the Leptomonas collosoma spliced leader RNA (L. collosoma SL RNA) of 56 nt, two experimental secondary structures 1 and 2 have been elucidated. Neither is the minimum free energy (MFE) structure computed by mfold server (http://www.bioinfo.rpi.edu/applications/mfold/). Based on structures generated by our sampling algorithm, sampling estimates for the MFE structure and the two experimental structures are computed (Table 1 in FIG. 6). The MFE structure has the largest observed frequency among all sampled structures. Furthermore, the Boltzmann equilibrium probability of a structure (equation (1) or (11)) is closely estimated by its maximum likelihood estimate (MLE) computed from the sample and is contained in the 95% confidence interval (CI). This gives an illustrative example of the theoretical assertion that the algorithm samples secondary structures by their Boltzmann equilibrium probabilities.

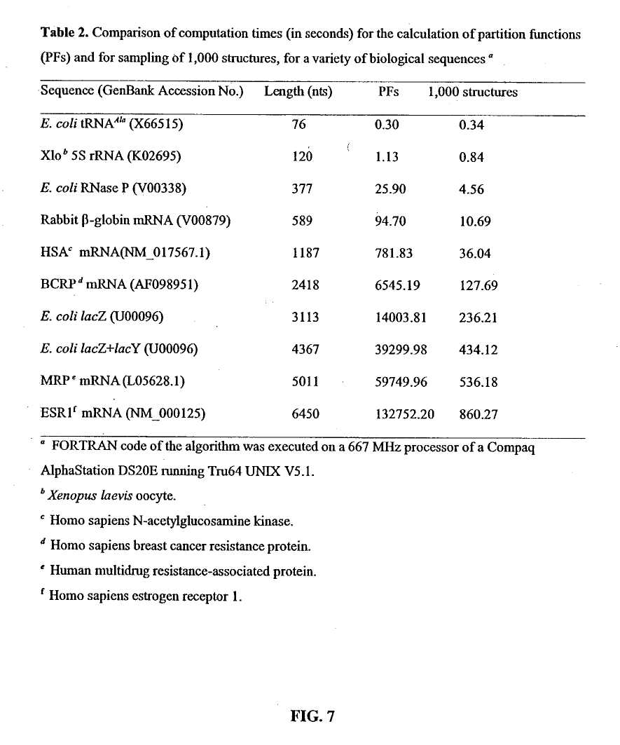

Because there are no more than (n−3)/2 base pairs in a secondary structure and the time for sampling a pair is at most O(n) when long interior loops are disallowed, the time of the sampling algorithm is bounded by Op(n²), i.e., stochastically quadratic in the worst case. Thus, once the forward recursions for the partition functions are completed in cubic time, a sample of structures can be quickly generated. This is illustrated by Table 2 in FIG. 7 for ten biological sequences having a wide range of lengths (the time for calculating partition functions can be perfectly fitted by a cubic curve; a figure is not shown here).

Class Representation of Boltzmann Ensemble of Secondary Structures

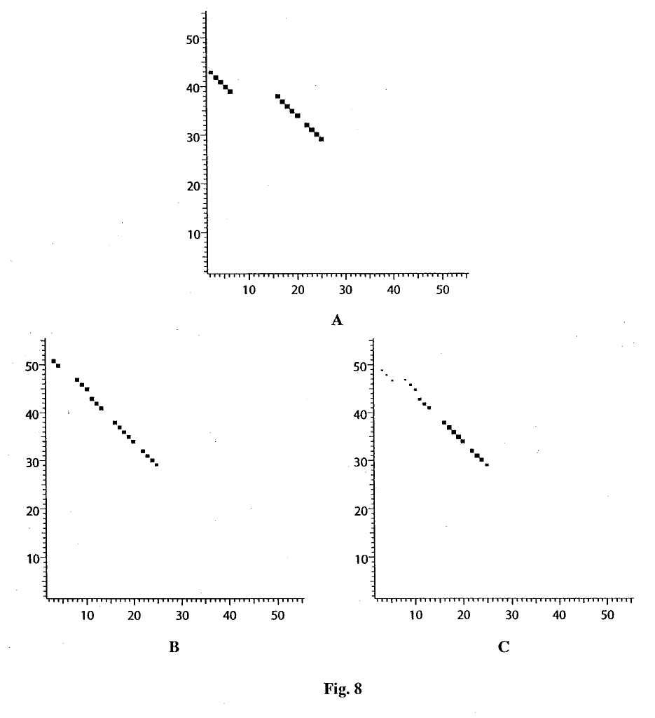

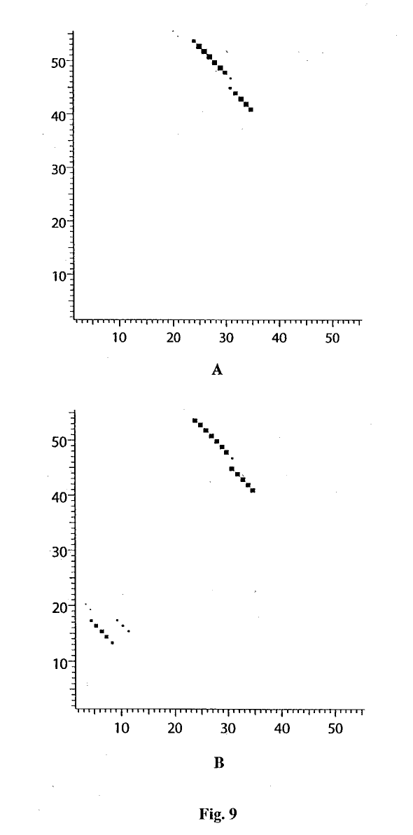

Classification of sampled structures. For the Leptomona collosoma spliced leader RNA (L. collosoma SL RNA), two competing secondary structural form 1 and 2 have been indicated by ribonuclease data, although the role of structures has yet to be identified. 1,000 structures sampled by our algorithm for this sequence of 56 bases were examined. It was found that the structures fall into two classes 1 and 2, corresponding to the two experimental structural forms 1 and 2. Class 1 can be further subdivided into classes 1A, 1B, and 1C; each of these subclasses has a yet higher level of structural similarity among its members. Class 2 can be further broken down into classes 2A, and 2B. A group of structures can be displayed by means of a two-dimensional histogram (2Dhist). Distinct patterns in this representation are indicative of common structural features for the group, whereas scattering of the squares would indicate its structural diversity. As illustrated by FIGS. 8A-C, structures in classes 1A, 1B, and 1C have in common two helices, represented by the two clusters of 5 squares and 4 squares, respectively. Specifically, the first helix is formed by base pairs U¹⁶!A³⁸, G¹⁷!C³⁷, A¹⁸!U³⁶, A¹⁹!U³⁵, and G²⁰!C³⁴. The second helix is formed by A²²!U³², C²³!G³¹, A²⁴!U³⁰, and G²⁵!C²⁹. On the other hand, the histograms also show that members of these classes have different structural features. Structures in classes 2A and 2B also have in common two helices (FIGS. 9A-B), which are different from the two common helices for classes 1A, 1B, and 1C. The major difference between class 2A and class 2B is the existence of an additional helix for class 2B. This helix is represented by a cluster of squares in the bottom left portion of the histogram in FIG. 9B.

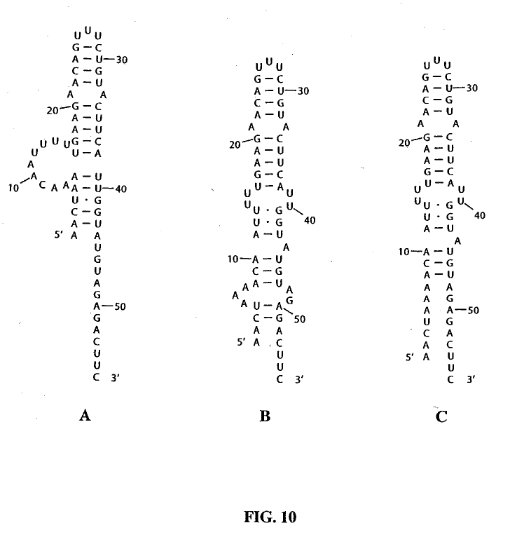

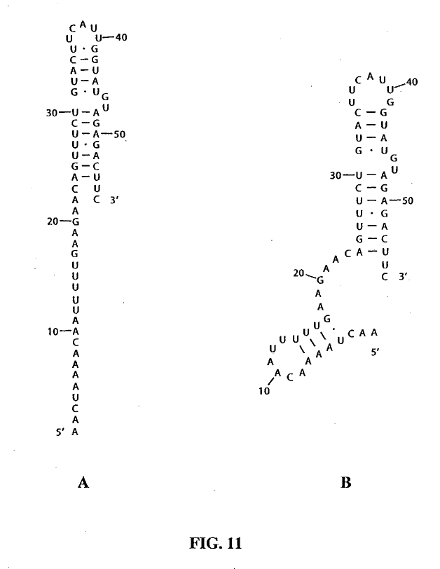

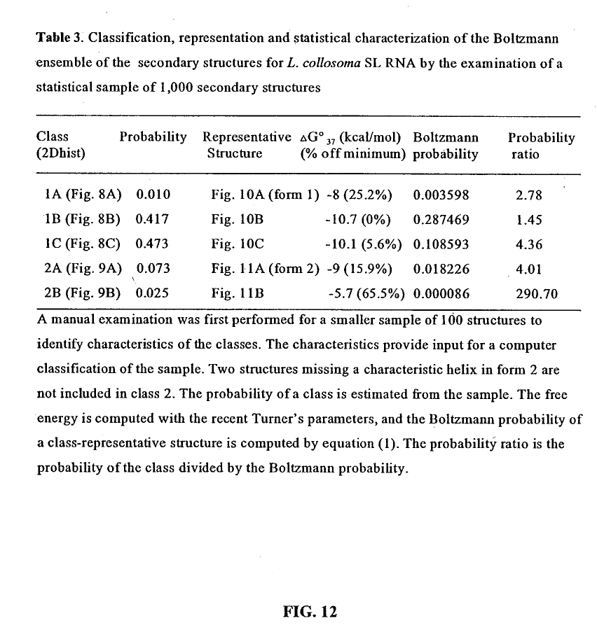

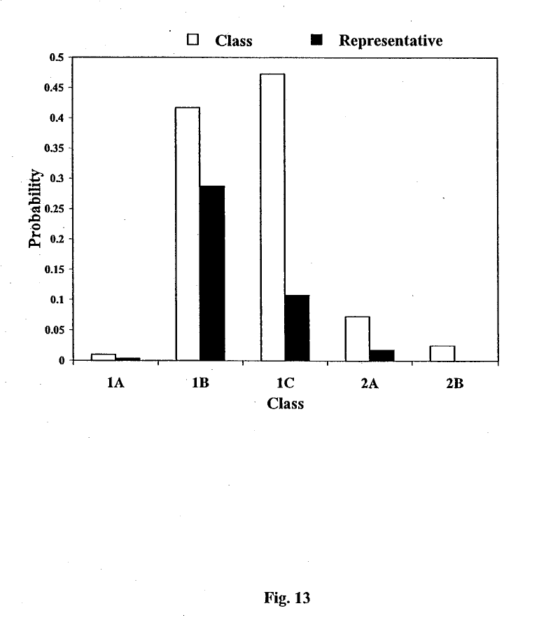

Probability of a class and the Boltzmann probability of its representative. For a class of similar structures, the structure occurring with the highest frequency (i.e., the most probable structure) in the sample is taken as the representative of the class. Class 1A is represented by experimental structural form 1 (FIG. 10A). The minimum free energy (MFE) structure from mfold shown by FIG. 10B is the representative of class 1B. Class 1C is represented by the structure in FIG. 10C that is the MFE structure with a short helix removed. Experimental structure form 2 (FIG. 11A) is the representative for class 2A. The representative for class 2B shown by FIG. 11B is experimental structural form 2 with an additional hairpin-helix stem on its long single-stranded 5′ end. The probability of a class is computed by its frequency in the sample, the Boltzmann equilibrium probability of the representing structure is computed by using its free energy, and the partition function available from the forward step of the algorithm (equation (1)). The size of a class is reflected by the class probability. It is a surprising observation that the Boltzmann probability of the representative structure is not necessarily reflective of the magnitude of the class probability (Table 3 in FIG. 12, FIG. 13). For example, the probability for class 1C is about 13.4% larger than that for class 1B; however, the Boltzmann probability of class 1C's representative is merely 37.8% that of the representative structure for class 1B.

“Entropic class”. For class 2B, the ratio of the class probability and the Boltzmann probability of its most probable member is 290.70, which is strikingly high. Despite the very small Boltzmann probability for its most probable member, this group contains a substantial number of similar structures such that the collection of these structures has a much higher aggregate probability. Such “entropic class” of structures can be revealed by sampling through classification. However, a structure in an entropic class can be easily overlooked when it is examined individually on the basis of its free energy or Boltzmann probability.

Table 3 in FIG. 12 presents a summary of the above analyses. Although the two experimental structures are 25.2% and 15.9% off the minimum free energy, respectively, they are both predicted by the sample. Version 3.1 of mfold (2) was run on mfold server (http://www.bioinfo.rpi.edu/applications/mfold) to fold this sequence. For suboptimality percentage P under 15 (default=5), only the optimal folding is returned. Only for a large P, e.g., P=30, the two alternative structures are returned as suboptimal foldings. This example underscores the importance of examining suboptimal structures. It also shows that important alternative structures and structural motifs can be revealed by a statistical sample of the Boltzmann ensemble. These findings suggest that the Boltzmann ensemble of RNA secondary structures can be more adequately represented by classes (through 2Dhist) taken with their probabilities, together with the class-representative structures and their Boltzmann probabilities. Thus, through structure classification, the sampling approach can achieve the objective of both efficient and statistically unbiased representation of suboptimal foldings.

Prediction of Alternative Structures

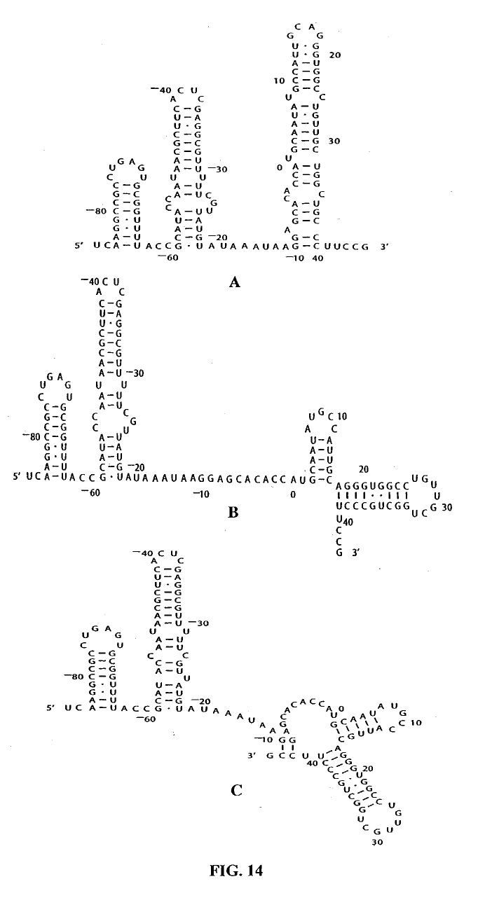

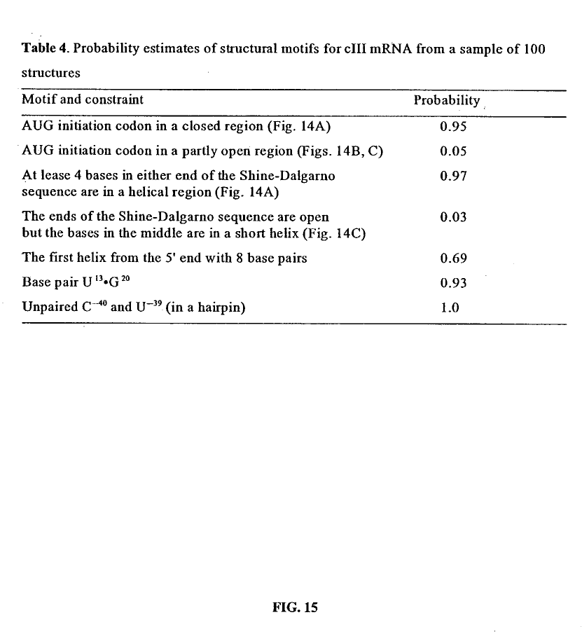

The analysis of L. collosoma SL RNA suggests that alternative biological structures can be adequately revealed by a statistical sample. This can be investigated further by applying the sampling algorithm to mRNA secondary structure prediction. mRNA secondary structures can play a regulatory role of determining the rates of translation initiation. This is explained by a model of coexisting alternative structures: one structure favors the translation initiation while the other inhibits the translation initiation. Also, it has been argued that the accessibility of the initiation codon is important for maximizing expression. The secondary structure of a mRNA is generally unavailable by experimental means, because complete structural probing by chemical or enzymatic methods is very difficult for long-chain RNAs. A rare exception is the short mRNA for cIII gene of bacteriophage 8, for which two conformations A and B (FIG. 14A and FIG. 14B) were elucidated and were demonstrated to coexist in equilibrium. The sequence of 132 nucleotides in the structures covers 46 nucleotides of the coding region and 86 nucleotides upstream from the initiation codon A⁰UG². In structure A, the initiation codon and part of the Shine-Dalgarno sequence UΓ¹³AAGGAGΓ⁷ are in a closed, base-paired conformation such that the ribosome binding site is occluded. In structure B, the ribosome binding site is open for interactions. It is speculated that the cIII gene expression is regulated at the translation initiation level by the ratio of the two structures at the equilibrium, and changes in temperature or Mg²⁺ concentration, and perhaps ribosome binding can shift the equilibrium.

For cIII mRNA, a sample of 100 structures was generated by the algorithm and was manually examined. In this sample, 89 are close variants of structure A. The left stem in structure A is precisely predicted in 67 of the 89 structures. The exact right stem and a modification with one or both of additional pairs AΓ¹²:U⁴², AΓ¹¹:U⁴¹ are predicted in 72 of the 89 structures. Appreciable variability in the location of interior and bulge loops is observed for the middle stem. Structure C in FIG. 14C is one of three structures in the sample which closely resemble structure B. The appreciable modification is the additional short helix involving the Shine-Dalgarno sequence formed by base pairs GΓ¹⁰:C⁴⁴ and GΓ⁹:C⁴³. The remaining eight structures (structures not shown) in the sample do not resemble either structure A or B and have diverse structural features. The optimal folding by mfold is a modification of structure A with three additional base pairs CΓ⁵⁴:GΓ³⁵, AΓ¹²:U⁴² and AΓ¹¹:U⁴¹, with the MFE of εGE₃₇=!48.5 kcal/mole. Structure A is well predicted by the optimal folding. Its free energy is εGE₃₇=!46.1 kcal/mole, 5% off the MFE. Structure B has εGE₃₇=!40.2 kcal/mole, 17% off the MFE. Structure C has εGE₃₇=!42.9 kcal/mole, 12% off the MFE. For P=30, neither B nor a variant resembling B as closely as C is predicted by suboptimal foldings from mfold, although both structures B and C are well within this range of suboptimality. By using the option for specifying base pair constraint in rfold, we verified that structures B and C are indeed in the “missing” set of suboptimal foldings that are excluded by the algorithm design for mfold. In contrast to the stability indicated by the free energies, experimental analysis showed that structure B is favored by a factor of about 3. The discrepancy could be explained by tertiary interactions which preferentially stabilize structure B. This application not only presents an example that an important alternative structure can be better predicted by a sample of moderate size, but also shows that alternative structures of low probability can be biologically important because stability contribution from potential tertiary interactions are unaccounted for. The finding also suggests the sampling algorithm can be well suited to the prediction of secondary structure of mRNAs, because an mRNA may have many conformations in an intracellular environment.

Assignment of Probabilities for Structural Motifs

In many applications, certain structural motifs are of biological interest. Sampling also enables probabilistic prediction of any motif with or without specific constraint(s). The probability of a motif can be directly estimated by the frequency of its occurrence in a sample. This is shown in FIG. 15 for several constrained motifs involving the AUG initiation codon or the Shine-Dalgarno sequence of CIII mRNA, and for a helix, a base pair and a single-stranded region of two bases. The algorithm by McCaskill is limited to the probability calculation for individual base pair and unpaired base. Probabilities of larger motifs such as helices of two or more base pairs and single-stranded regions of two or more unpaired bases are not available from this algorithm. In contrast, the sampling algorithm is readily applicable for this purpose.

Boltzmann-Probability-Weighted Density of States and Free Energy Distributions

Cupal et al. presented a recursive algorithm to compute the free energy distribution of all secondary structures (i.e., density of states (DOS)). The algorithm is O(n⁵) in time with a memory requirement of O(n³), and is thus computationally prohibitive even for sequences of moderate length. For short sequences, this algorithm is useful for the study of evolution by comparison of DOS between biological sequences and random sequences of the same composition (Higgs).

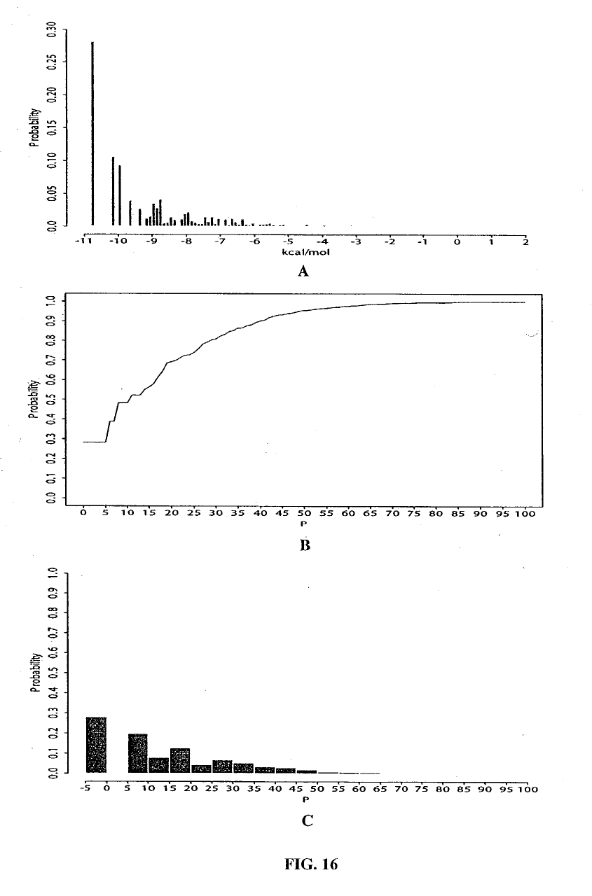

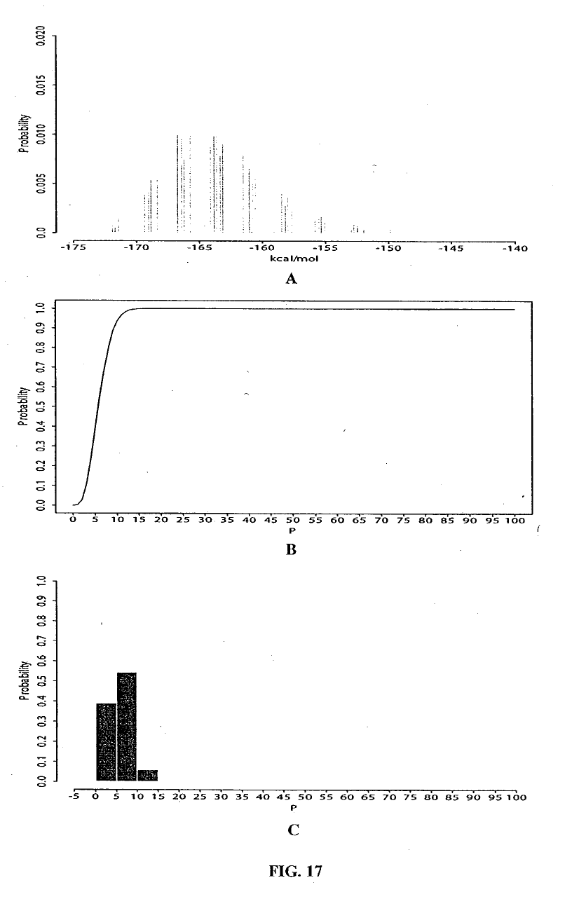

The free energy distribution of probable structures for either short of long sequence is available from our sampling algorithm and is referred to as the Boltzmann-probability-weighted density of states (BPWDOS) (FIGS. 16A, 17A). Information for the BPWDOS can be displayed in alternative ways for showing the probability that the free energy of a structure is within a threshold of the global minimum or is in an energy interval (FIGS. 16B, 16C, 17B, 17C). Sampling also generates representative structures for a given low energy interval. This overcomes the disadvantage of the algorithm by Cupal et al. that there is no information about individual structures corresponding to the low energy states. These distributions could be valuable for evolutionary studies on long sequences and studies on the RNA energy landscape (Schuster & Stadler).

The sampling algorithm in accordance with the invention is shown to be an appealing alternative to existing algorithms for RNA secondary structure prediction. A sample from the Boltzmann distribution can adequately delineate the Boltzmann ensemble of secondary structures through classification. This approach avoids the limitation of suboptimal folding presentation by a designed set and the difficulty with a complete enumeration of suboptimal foldings. The algorithm is shown to meet the challenge of predicting alternative structures. The prediction of structural motifs can be useful in applications. A promising application to antisense target prediction by the probabilities of single-stranded regions will be described in further detail below. The sampling approach of the present invention is also powerful tool for some important RNA research problems. The capability of predicting alternative structures suggests sampling can be a promising method in the application to the prediction of conformational switch, a phenomenon involved in translational regulation, transcriptional attenuation in prokaryotes, translocation process, protein biosynthesis, viral regulation, etc. Because an algorithm according to the present invention implicitly simulates folding pathways according to statistical mechanics principle, this approach may allow for adequately characterizing sequential folding and folding pathways and revealing metastable states into which an RNA can be trapped during folding. The classes may correspond to different folding pathways. Sampling may also provide a tool for statistical delineation of the free energy distribution (i.e., the density of states up to a proportionality constant) of the Boltzmann ensemble, and a test to determine if this distribution follows a certain pattern(s) and if it displays two local minima in the case of conformational switch. An algorithm may be O(n³) by disregarding long interior loops. Under an assumption on interior loop asymmetry, an approach has been developed to reduce the time of interior loop evaluation from O(n⁴) to O(n³). The sampling stage of the algorithm may be implemented using, e.g., Fortran 77, on a computing device such as system 100. It is noted that an algorithm in accordance with the present invention may be programmed in any computing device or implemented by designing any type of dedicated hardware for performing the steps thereof. For an RNA sequence of 589 nucleotides, it takes 102 s to complete the partition function calculation, and 87 s to generate 1,000 structures on a 300 MHz CPU of a Ultra 2 Scalable Performance Architecture (“SPARC”) workstation. Manual classification of structures can be performed for a sample of moderate size. An automated procedure for classifying large number of structures may be used to fully take advantage of the sampling approach. While coaxial stacking interactions might not be included in a rigorous dynamic programming algorithm, a recalculation of the free energies of suboptimal structures has been proposed to incorporate coaxial stacking for multi-branched loops. Similarly, a resampling scheme that includes an energy reevaluation step for sampled structures and a resampling step of these structures based on modified free energy values may accommodate coaxial stacking.

Probability Profiling for Predicting Single-Stranded Regions in RNA Secondary Structure

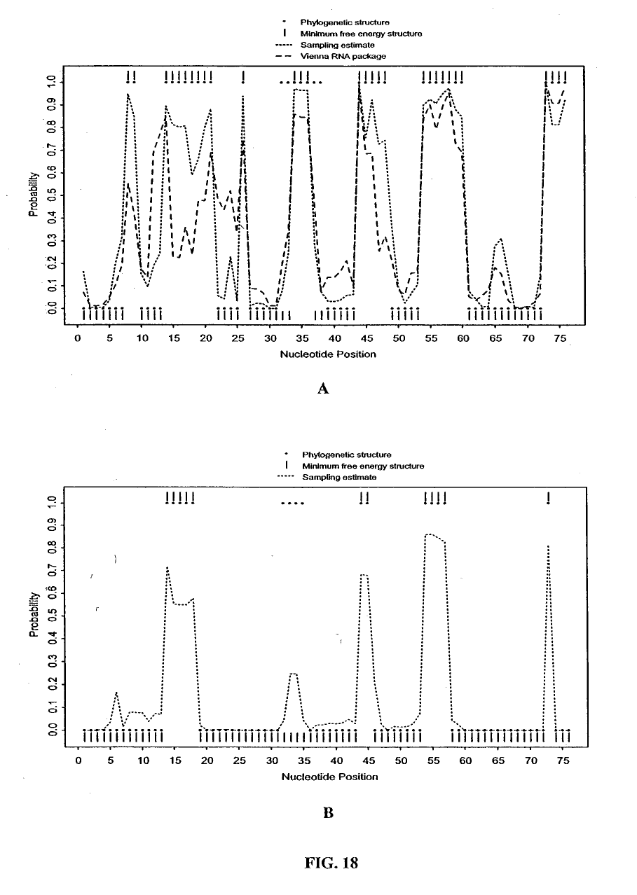

For single-stranded bases in E. coli tRNA, FIG. 18A demonstrates a probability profile estimated from 1,000 sampled secondary structures according to the present invention, the probability profile computed by the Vienna RNA package, the profile indicated by the minimum free energy (“MFE”) structure computed with version 3.1 of mfold (http:/www.bioinfo.rpi.edu/applications/mfold/), and the one indicated by the phylogenetically determined structure. A sample size of 1,000 was found to be adequate because the profile estimates from this sample and a larger sample of 10,000 structures were not readily distinguishable. For the unpaired individual bases, the probability profile and the profile by the MFE structure are comparable. This is generally expected because the MFE structure is the most probable structure in the sample. However, the MFE structure substantially underpredicts the width of the region around nucleotide G³⁵ of the anticodon loop, while a significant portion of the sample by the present invention adequately reveals the width. For the region between nucleotide G³⁰ and A⁷⁶, the sampling approach and the latest version 1.3.1 of the Vienna RNA package gave comparable results; however, for the region between nucleotide C⁵ and C²¹, the sampling profile by the present invention predicted the phylogenetic structure substantially better than the Vienna profile. The current version of Vienna package is based on an earlier compilation of Turner's free energy parameters. It has been shown that the latest update improves the prediction of secondary structure. This explains the better performance by the sampling algorithm of the present invention.

FIG. 18B shows the probability profile of a sample for single-stranded sequences with a sequence width of four nucleotides. For comparison with the phylogenetic structure, a dot with coordinates (i, 1) is shown in FIG. 18B if the four nucleotide sequence starting at nucleotide i is single-stranded, and a dot with coordinates (i, 0) is plotted if any of the four nucleotides is base paired. Similarly, the MFE structure is plotted. The unstructured region of the anticodon loop is missed by the MFE structure, but is revealed by the sampling profile through a peak of substantial probability. For the two sampling profiles in both FIG. 18A and FIG. 18B, not only do the single-stranded regions in the phylogenetic structure correspond well to the local peaks of the probability profiles, but also the width of the regions matches the width of the peaks with only one exception, region ³²AUGGCAU³⁸ of the anticodon loop. The peak for this region in the phylogenetic structure is slightly narrower because two Watson-Crick pairs A³²-U³⁸ and U³³-A³⁷ are likely to be predicted by any free-energy-based algorithm, while these two base pairs are absent in the phylogenetic structure. In FIG. 18B, the peak of the sampling profile between A³² and U³⁸ is much lower than the corresponding peak in FIG. 18A because, while the single-stranded probability for each of G³⁴, G³⁵, and C³⁶ is over 0.96, the probabilities for U³³ and A³⁷ are below 0.28. Thus, for identifying a single-stranded region of at least four nucleotides, a high peak in the profile of single-stranded bases can be visually misleading when the width of the peak is smaller than 4 nucleotides. The probability profile of single-stranded sequences presents a clearer picture of potential antisense sites, because it has fewer and narrower peaks than the profile of single-stranded bases. This probability profile cannot be obtained by the Vienna RNA package or other existing computational methods.

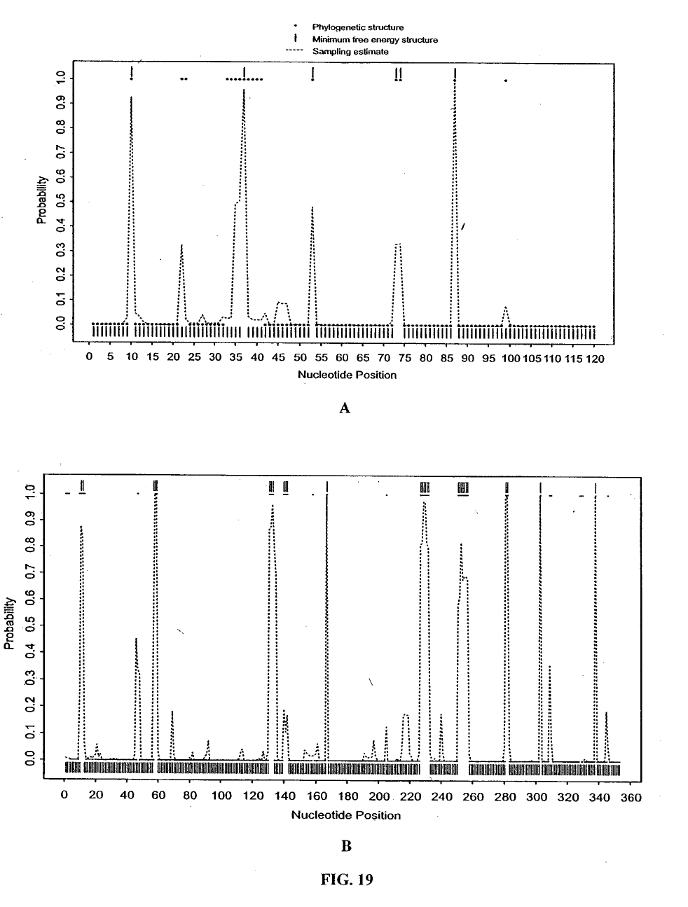

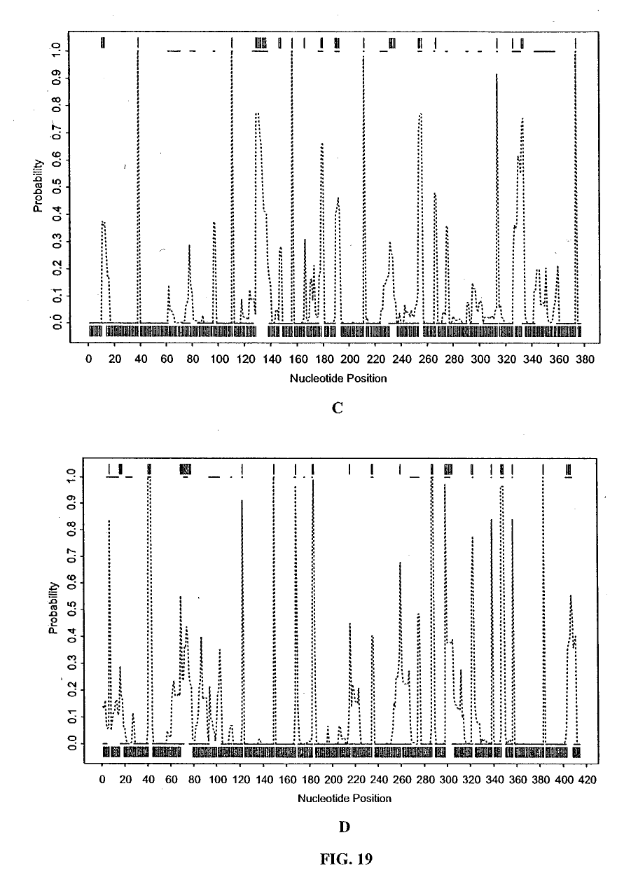

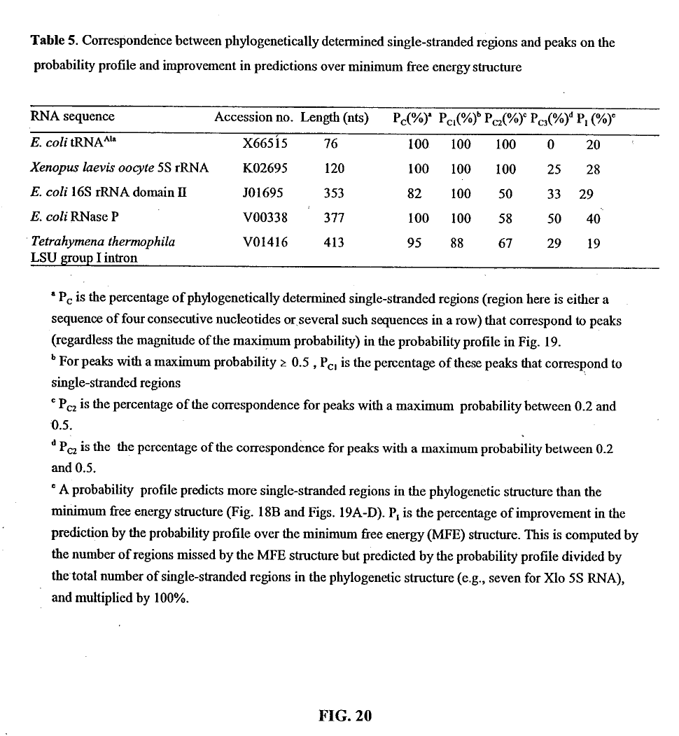

To further illustrate the sampling approach of the present invention, probability profiles in FIGS. 19A-19D are presented for the following representative RNA sequences with phylogenetically determined secondary structures: Xenopus laevis oocyte (“Xlo”) 5S rRNA, domain II of E. coli 16S rRNA, E. coli RNase P, and group I intron from 26S rRNA of Tetrahymena thermophila. For these sequences, phylogenetically determined single-stranded regions correspond to peaks in the probability profile with near certainty (FIGS. 19A to 19D) (P_{C} in Table 5 of FIG. 20). On the other hand, peaks with at least a maximum probability of 0.5 almost certainly point to single-stranded regions (P_{C2}, in Table 5 of FIG. 20); peaks with a maximum probability between 0.2 and 0.5 have at least a 50% chance of correctly indicating single-stranded regions (P_{C3} in Table 5 FIG. 20), whereas there is a far smaller but appreciable chance for peaks with a maximum probability under 0.2 (P_{C3} in Table 5 of FIG. 20) to correctly indicate single-stranded regions. As in the case of E. coli tRNA^{Ala}, for all of these RNA sequences, the probability profile reveals more single-stranded regions in the phylogenetic structure than the MFE structure (P_{I} in Table 5 of FIG. 20). The substantial improvement is because the alternative structures in the sample by the present invention are able to reveal structural motifs not predicted by the MFE structure. On the other hand, the motifs in the MFE structure are well reported by the sample because the MFE structure is the most probable structure in the sample. The improvement is noticeably greater for E. coli RNase P, which has highest percentage of nucleotides in pseudoknots, a motif not allowed by either mfold or the algorithm according to an exemplary embodiment of the invention.

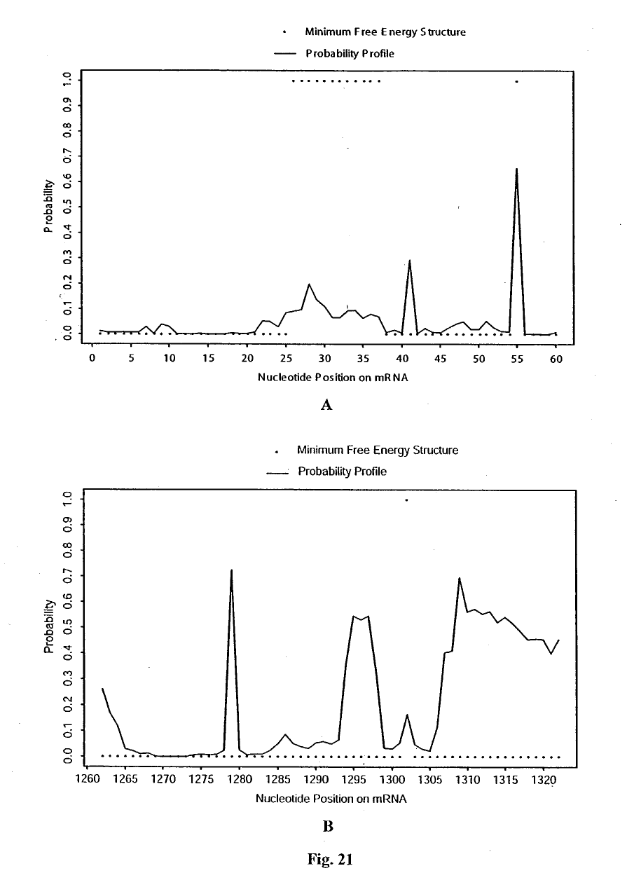

The results reveal variation in the reliability of prediction among different RNAs. For free energy minimization for the prediction of RNA secondary structure, variability in the reliability of prediction for different RNAs has been well documented. Because the sampling algorithm of the exemplary embodiment of the invention is also based on free energies, it is not surprising to observe a similar phenomenon. There is also substantial variability in the maximum probabilities for the peaks that correspond to single-stranded regions. Similarly, for minimum free energy prediction of secondary structure, there is variability in the reliability of predictions for different regions of a sequence. The summary in Table 5 of FIG. 20 indicates that single-stranded regions predicted by high probability peaks are “well-determined” by the probability profile. In other words, these regions are highly stable and thus are present with high probability in a sample of probable secondary structures. For regions of lower stability, their probabilities are either moderate or low, because alternative structural motifs will be more likely to be present in the sample. The sampling algorithm of the exemplary embodiment gives a complete statistical presentation of probable competing alternative structures. Thus, the probability profile provides a statistical delineation of single-stranded regions with varying stabilities. Furthermore, by assigning probabilistic confidence measure in predictions, new accessible sites can possibly be identified, as illustrated by FIG. 21.

Antisense Application

The rabbit β-globin mRNA (589 nt, GenBank accession V00879, coding region 54-497) has been well studied for antisense inhibition of protein synthesis. An 11-mer and three 17-mers have been used to target rabbit β-globin mRNA in a wheat germ extract as well as in microinjected Xenopus oocytes. The inhibition of cell-free translation by eight phosphodiester antisense oligonucleotides (“ASO”s) targeted to this mRNA has been examined. A combinatorial oligonucleotide array technique for hybridization assessment of oligonucleotides within a given region has also been used. For the rabbit β-globin mRNA, an array of 1,938 oligonucleotides up to a length of 17 bases, has been used to measure the ASO:mRNA hybridization potential. These oligonucleotides were complementary to the first 122 bases of the mRNA. Three oligomers, BG1, BG2, and BG3, were chosen for study by in vitro translation in wheat germ extract and the RNase H assay.

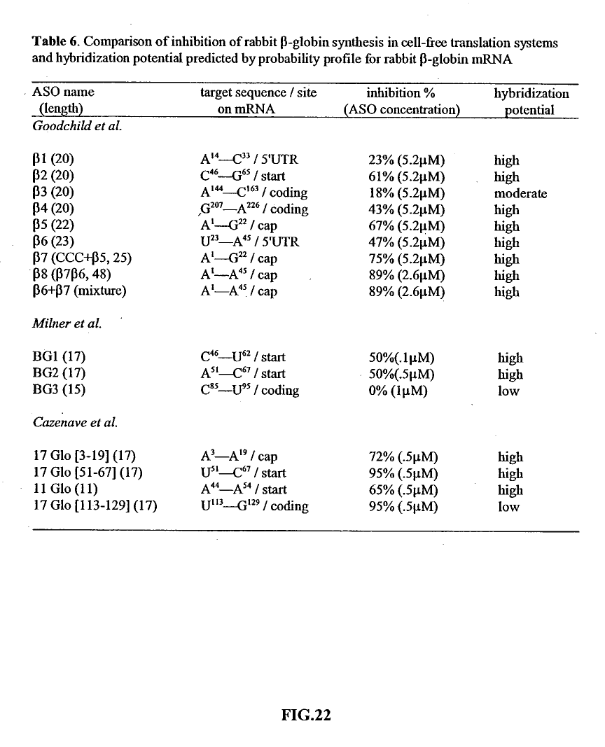

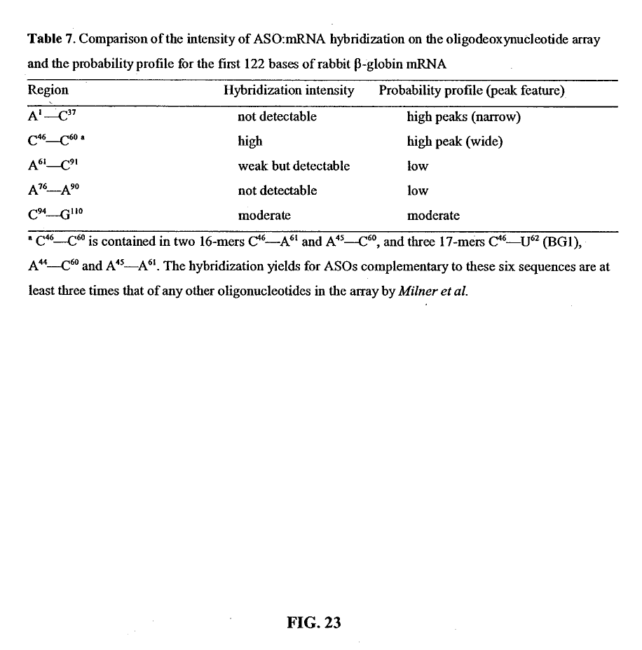

In an analysis, the results for BG1, BG2, and BG3 are directly compared to the data from the other two groups, because all these ASOs were studied in cell-free translation systems and the percentages of translation inhibition were reported (Table 6 in FIG. 22). The inhibition percentages facilitate a quantitative comparison and assessment of the correlation between inhibition of cell-free translation and computational predictions. Qualitative array hybridization data and the computational predictions were summarized and compared separately (Table 7 in FIG. 23).

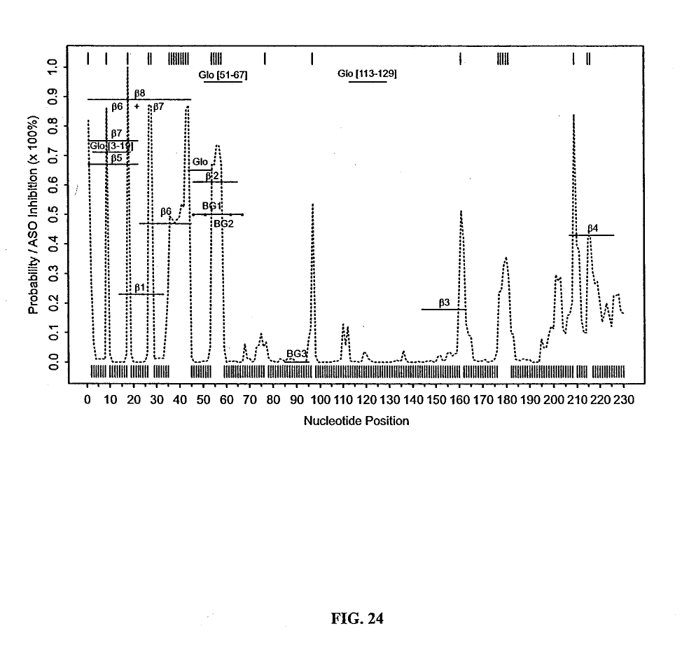

The probability profile with a sequence width of four nucleotides was computed with a sample of 1,000 secondary structures for the rabbit β-globin mRNA. The probability profile and the profile by the MFE structure for the region A¹-U²³⁰ are shown in FIG. 24, as the ASOs in these studies were targeted to this part of the mRNA. The target sites on the mRNA, the inhibition effect in cell-free translation systems in the three studies, and the hybridization potential predicted by the probability profile are summarized in Table 6 in FIG. 22. For this analysis, the hybridization potential was assessed as high if, for the target site, there was at least one peak with probability≦0.6; the potential was considered moderate for a peak with probability between 0.3 and 0.6; the potential was low for a site with a probability under 0.3 of being partly single-stranded. For ASOs in Cazenave et al., the inhibition figures for wheat germ extract were estimated from FIGS. 3 and 7 in Cazenave et al. The region A¹-A⁴⁵ was targeted by five of eight ASOs in Goodchild et al. There are three high probability sequences in this region: A¹-C⁴, A¹⁸-U²¹, and U³⁶-A⁴⁵. They explain the predicted high hybridization potential for β5, β6, β7, β8, and β6+β7. The moderate inhibition by β1 indicates that A¹⁸-U²¹ alone is not as effective as the other two. One explanation is that the two adjacent nucleotides, C¹⁷ and G²², are predicted to almost certainly engage in G×C pairing, and thus they might present a substantial energy barrier for hybridization elongation by “zippering”. The high inhibition by β8 and β6+β7 suggests that an antisense effect can be enhanced by simultaneously targeting several high potential sites. Consistent results for BG1, BG2, and BG3 were found in Milner et al. Clear inconsistency between the predictions by the present invention and the observed inhibition was found for 17 Glo [113-129] of Cazenave et al, which appears to be an exception to the rule of thumb of at least four unpaired bases. In the case of an effective antisense site with less than four unpaired bases, the site would not be predicted by the probability profile with a sequence width of four nucleotides. On the target site of 17 Glo [113-129], the probabilities of being unpaired for U¹²⁵ and G¹²⁶ are 0.61 and 0.56, respectively, but the probabilities are less than 0.1 for adjacent bases U¹²⁴ and G¹²⁷. Among many other potential reasons for poor prediction in this case could be tertiary interactions and RNA-protein interactions, and self-folding of the oligomer that are unaccounted for by the current algorithm.

If low, moderate, and high hybridization potential are associated with inhibition of 0-19%, 20-39%, and 40-100%, respectively, then for 13 of the 16 ASOs (81%) examined, the hybridization potential revealed by the probability profile is indicative of the antisense inhibitory effect. For all the ASOs, there is a significant correlation (P value=0.0147, correlation coefficient=0.597) between the hybridization potential predicted by the probability profile and the degree of translation inhibition. For β1-β8, there is a substantially higher correlation (P value=0.0037, correlation coefficient=0.882). In contrast, Stull et al. found no significant correlations between observed inhibition and any predictive indices for β1-β8. For ASOs in Cazenave et al., Stull et al. found a correlation between Dscore, one of their indices, and inhibition for oligomer concentration at 6 μM, but no significant correlation for oligomer concentrations below 6 μM. The probability profile and the MFE structure give comparable predictions of single-stranded regions. However, without an associated measure of confidence, there is a lack of correlation between the binary prediction by the MFE structure and the degree of translation inhibition (P value=0.567, correlation coefficient=0.155). This exemplifies the observation that there is limited success in using MFE structure for antisense design. Because the sampling profile provides a statistical measure of confidence in the predictions, it is not surprising that the profile is found to be generally indicative of the degree of translation inhibition.

For the hybridization intensity data in Milner et al., there is very good agreement between the hybridization intensity and the probability profile for regions C⁴⁶-C⁶⁰, A⁷⁶-C⁹⁰, and G⁹⁴-G¹¹⁰ (Table 7 in FIG. 23). The hybridization intensity for region A⁶¹-C⁹¹ is in reasonable agreement with the probability profile. In this region, the maximum probability of a peak is about 0.1. For a peak with a maximum probability under 0.2, there is an appreciable chance for the peak to correctly predict single-stranded regions (Table 5 in FIG. 20). Thus, weak hybridization is possible for low but appreciable probabilities. For region A¹-C³⁷, it is an intriguing contrast that the hybridization data are in disagreement with both the data in Goodchild et al. and the probability profile, but the probability profile is in good agreement with the data in Goodchild et al. The length of the oligomers, 20-45 nt in Goodchild et al, and at most 17 nt in Milner et al, offers an explanation for the conflicting results. Goodchild et al. indicated that a greater inhibition could be obtained by covering a longer portion of the mRNA. This is evidenced by the greater inhibition of β8 or a mixture of β6 and β7 than either β6 or β7 alone (Table 6 in FIG. 22). There are several sharp peaks in the probability profile for this region. Thus, a plausible explanation from the profile is that substantially longer ASOs cover more peaks in this region, and hence, enhance the chance of both nucleation and propagation of duplex formation. Although oligomer length has a positive effect on translation inhibition in this case, this may not be generally true. It is also noted that the conclusion of insignificant hybridization by Milner et al for region A¹-C³⁷ appears to be based on the lack of a continuous subregion with detectable hybridization. In this region, there are two isolated intensity bands in FIG. 1 of Milner et al., indicating substantial hybridization at sequence positions that were also targeted by Glo [3-19] of Cazenave et al.

The six oligomers containing bases C⁴⁶-C⁶⁰ (Table 7 and footnote in FIG. 23), and BG2, β2, Glo [51-67] in Table 6 in FIG. 22 share one common feature on the profile: a relatively wide, high probability peak between A⁵⁴ and U⁵⁸, with ⁵⁴AUG⁵⁶ being the initiation codon. This suggests that a smooth and relatively wide peak on the probability profile can be a high potency antisense site because the chance of hybridization is improved for a wider single-stranded region.

Rational Design of Antisense Oligos

Quantification of Nucleation Potential. Because a predicted site can be targeted by numerous oligos of the same length, and by many more with varying length, a quantitative measure of the nucleation potential is necessary for efficient oligo screening. A sampling-probability-weighted binding energy for measuring the binding affinity for nucleation, ΔG_{nucleation}, can be computed to address this issue. For the targeted sequence of m ribonucleotide 5′-r₁r₂. . . rₘ−3′ and the antisense oligo of m deoxynucleotides 3′-d₁d₂. . . dₘ−5′, this quantitative measure of nucleonation potential is computed by

ΔG_{nucleation}=ΔGᵢₙᵢₜᵢₐₜᵢₒₙ+Σ_{1≦i≦m-1}PᵢΔG_{stacking(i)}

where ΔG_{stacking(i)} is the energy for the ith RNA/DNA base pair stack and ΔGᵢₙᵢₜᵢₐₜᵢₒₙ is the free energy for duplex initiation, and Pᵢ is the probability that rᵢ is unpaired in the secondary structure of the target. The thermodynamic parameters for RNA:DNA hybrid duplexes (Sugimoto et al.) are used in the calculation. The probability Pᵢ is computed by our RNA structure sampling algorithm.

In the case that the target sequence is completely single-stranded with certainty, ΔG_{nucleation} is simply the sum of the initiation energy and the stacking energies for the RNA:DNA hybrid, because all of the weighting probabilities are 1. If every base in the target sequence is base paired with certainty, then all the weighting probabilities are 0, and ΔG_{nucleation} is ΔGᵢₙᵢₜᵢₐₜᵢₒₙ=3.1 kcal/mol. In this case, nucleation is energetically unfavorable. Through the thermodynamic parameters, GC content is accounted for indirectly in the calculation of ΔG_{nucleation}. More importantly, the uncertainty in the prediction of local structure at the target site is addressed through the weighting probabilities.

The results with rabbit β-globin mRNA suggest that relatively wide, high probability peaks on the probability profile are very likely to be effective antisense sites. The probability profile approach of the present invention offers a comprehensive computational screening of the entire mRNA or viral RNA. For several other mRNA sequences with length ranging from 1 kb to 3 kb, fifteen to twenty high hybridization sites per kb (data not shown) have been observed. These sites provide ample opportunities for rational design of antisense oligomers. An antisense oligomer is the reversed complement of a target sequence. The identification of optimal oligomers could be particularly important for antisense drug development. In applications, one can focus on sites within a particular mRNA region (e.g., coding region) of interest. In designing antisense oligomers, some basic rules are applicable for avoiding non-antisense effects and for enhancing antisense potency. Four Gs in a row should be avoided. To minimize the possibility of binding to a non-targeted mRNA with strong sequence homology at the binding site, a BLAST search for a prospect oligomer can be performed to ensure no appreciable overlap with other mRNAs in the experimental system. In particular, investigators need to be aware that translation initiation sites can have good homology in both related and non-related genes. To avoid stable intra-molecular structure within oligomers, oligomers that contain self-complementary regions (i.e., palindromic sequences) should not be used. Other experimental guidelines may also be used.

Rational Antisense Design. Based on probability profiling, a rational design procedure may be adopted for rational selection of antisense oligomers:

1. Computation for the construction of the complete probability profile of the target RNA.

2. Selection of accessible sites predicted by high probability peaks on the profile.

3. Select the antisense oligos (e.g., 20-mers) for each accessible site with the strongest probability-weighted-binding energy calculated with RNA:DNA stacking energy parameters.

4. Avoidance of three contiguous Gs, a motif known to cause non-specific effects.

5. Performing alignment search (e.g., BLAST) to avoid significant homology to other genes in the experimental system.

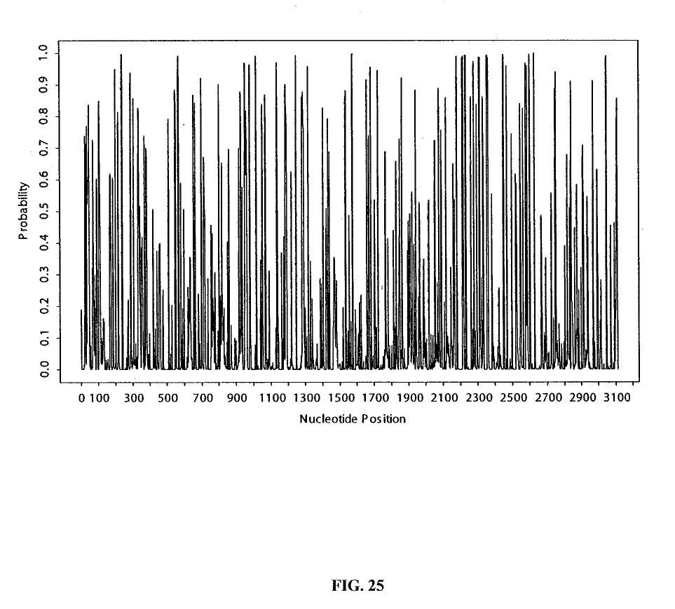

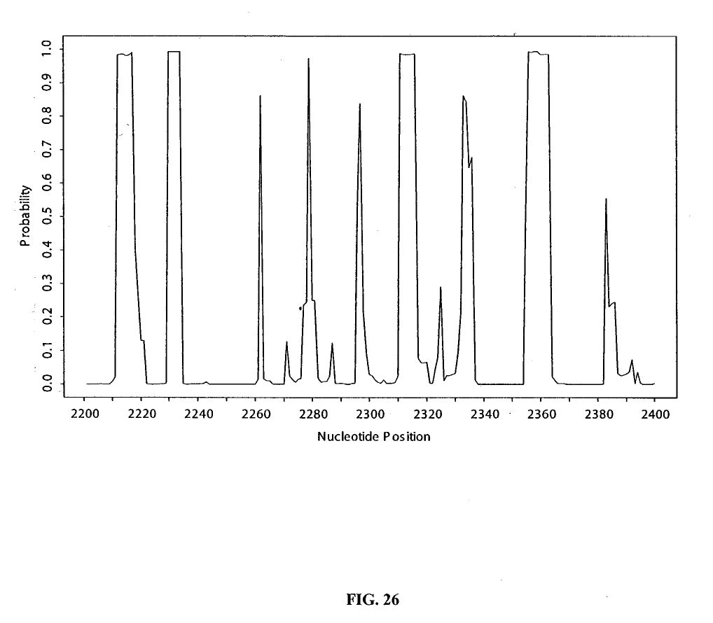

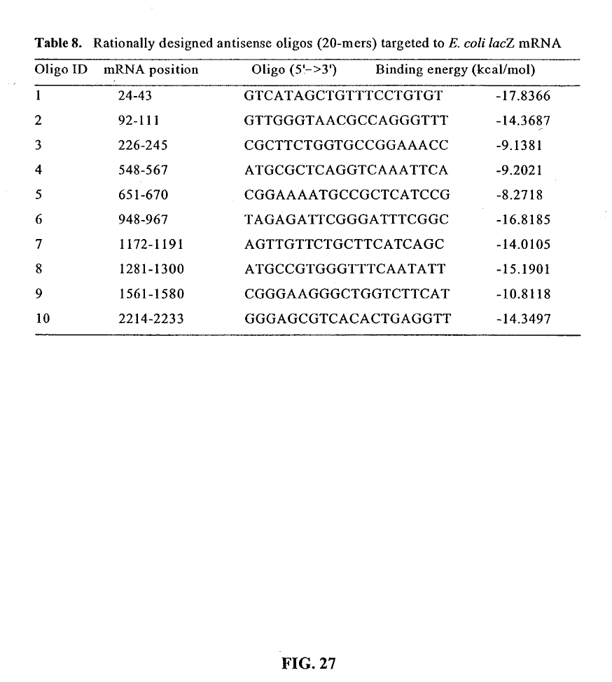

Example of antisense design. For E. coli lacZ (GenBank Accession No. U00096), which codes for β-galactosidase, the complete profile reveals 20 or so “well-determined” high antisense potential sites per kilobase (FIG. 25). A close-up examination of any region of the target can be facilitated by a zoomed-in version of the profile (FIG. 26, for nt 2200 through nt 2400). Ten antisense 20-mers were selected from the above design steps, and are listed in Table 8 of FIG. 27.

Mutual Accessibility Plot for Predicting RNA:RNA Interaction

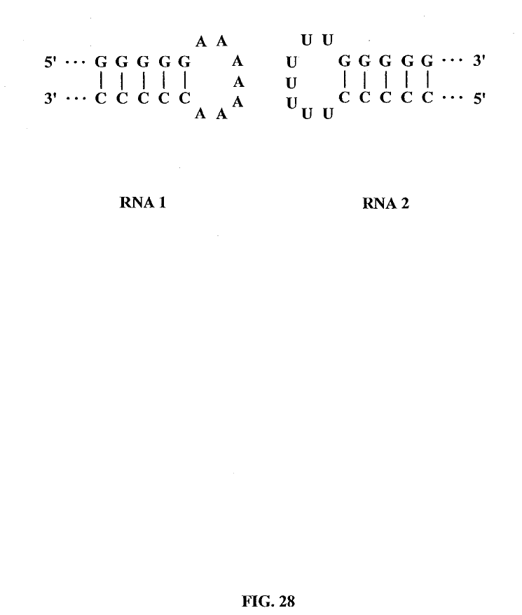

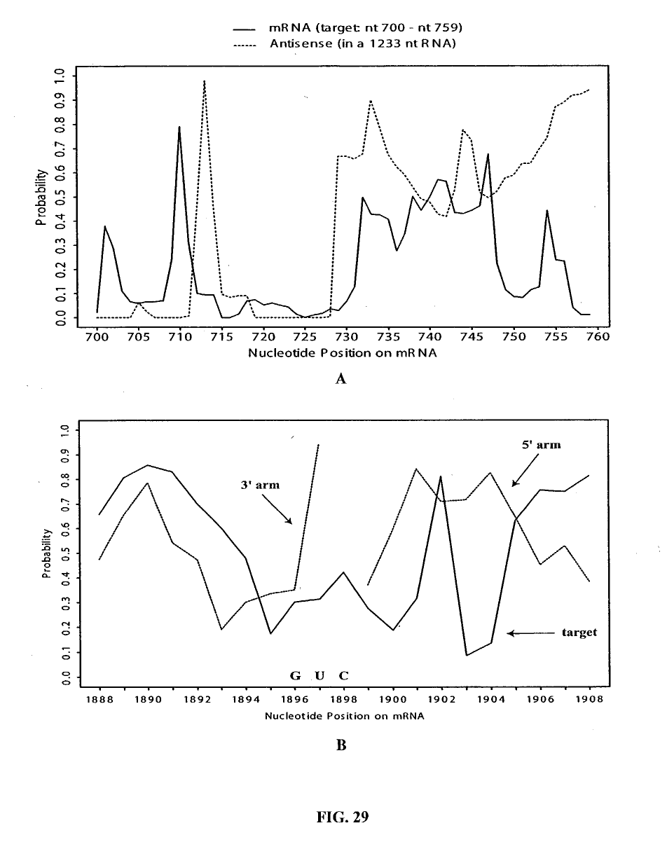

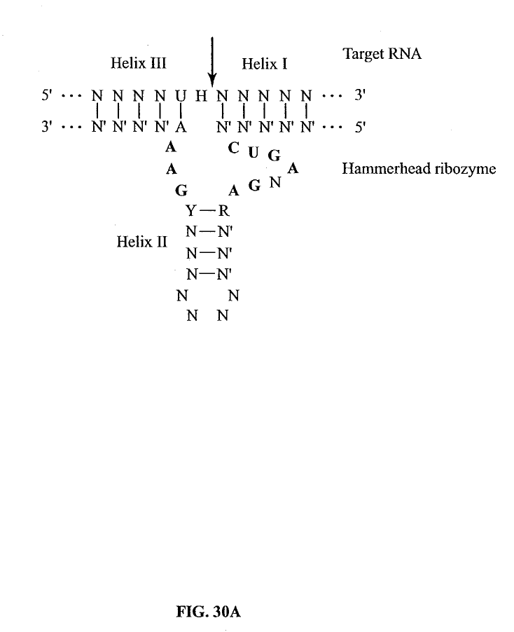

For RNA:RNA interactions through antisense binding, e.g., between RNA target and chemically synthesized or naturally occurring antisense ribonucleic acids (antisense RNAs), or between RNA target and trans-cleaving ribozymes, the structures of both RNAs are important. Thus, as illustrated by FIG. 28, antisense binding is largely dependent on the accessibility of both the bases on the target site and their complementary bases on the antisense RNA or ribozyme. This mutual accessibility between two RNAs can be assessed with an overlay plot of probability profiles for the two RNAs at the target site (FIG. 29). The mutual accessibility plot thus provides a new tool to address local accessibility of both RNAs at the site of interaction.

Rational Design of Trans-Cleaving Ribozyme

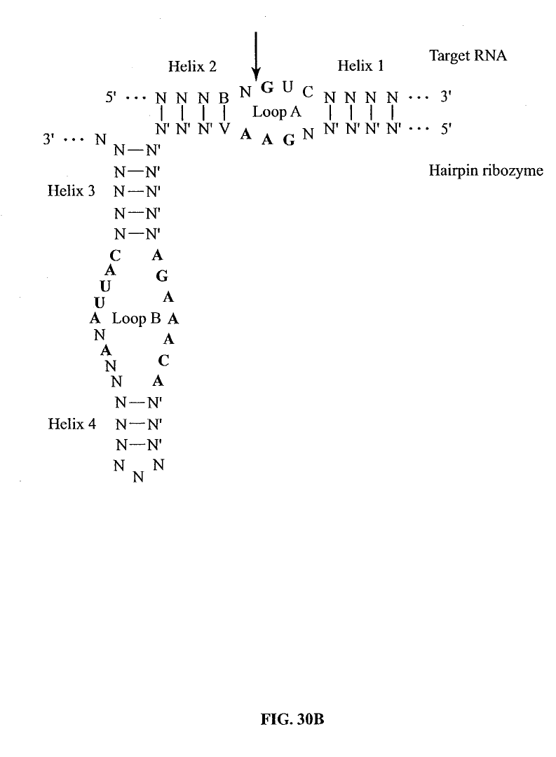

For trans-cleaving ribozymes (e.g., hammerhead or hairpin ribozyme, as illustrated by FIGS. 30A, 30B), the binding by the ribozyme's antisense arm(s) is the rate-limiting step. Thus, identification of accessible regions on the target is important for ribozyme design. On the other hand, for a hammerhead ribozyme, the two binding arms of need to be also accessible for interaction with target sequences flanking the cleavage triplet, e.g., GUC. The mutual accessibility between the target RNA and a ribozyme can be assessed with an overlaid plot of probability profiles at the target site (FIG. 29). The structure of the ribozyme is equally important. It has been an open issue to what extent incorrect ribozyme folds can be tolerated. The answer to this question may partly depend on the equilibrium between the correct ribozyme fold and alternatives (Christoffersen et al.). A probabilistic measure of this equilibrium calculated through classification of sampled structures for the ribozyme may be a good indicator for appropriateness of the catalytic domain of the ribozyme.

Rational ribozyme design. Based on probability profiling for both the target RNA and the ribozyme, and statistical folding of the ribozyme and subsequent structure classification, the following steps may be involved in rational design of trans-cleaving ribozymes:

1. Computation for the construction of the complete probability profile for the target RNA.

2. Evaluation of accessibility of both the cleavage site (e.g., GUC for hammerhead ribozyme) and its flanking sequences.

3. Specification of the bases of the ribozyme binding arms and subsequently the ribozymes for accessible sites.

4. Computation of the probability profile for each designed ribozyme.

5. Evaluation of accessibility of the ribozyme binding arms.

6. Evaluation of appropriateness of the structure of the catalytic domain of the ribozyme by structure classification for estimating the equilibrium between correct fold and alternatives.

7. Evaluation of mutual accessibility between the ribozyme binding arms and their target sequences.

Example of ribozyme design. The flanking sequences of all 23 GUC triplets for the breast cancer resistance protein (BCRP) mRNA (2418 nt, GenBank Accession No. AF098951) were analyzed for accessibility by probability profiling. For five of these sites, both flanking sequences are predicted to be accessible. For one of the five sites, nt 1896-1898 on the target mRNA, the resulting ribozyme has good mutual accessibility for both binding arms as illustrated by FIG. 28B).

Rational Design of SiRNAs

A probability-weighted-binding energy for the hybridization between the antisense strand siRNA and its complementary sequence on the target can be computed. The calculation is the same as the calculation of nucleation potential for antisense oligos with the only exception that RNA:RNA stacking energy (Xia et al.) is used here for RNA:RNA hybridization. Coupled with probability profiling for accessibility and other considerations, a rational selection process of siRNAs may involve the following steps:

1. Computation for the construction of the complete probability profile of the target RNA.

2. Selection of accessible sequences (e.g., AA(N19) motifs, where N is any nucleotide) of desired length (e.g., 21-23 nt) on the target.

3. Computation of probability-weighted-binding energy with RNA:RNA stacking energy parameters for the duplex formed between each selected target sequence and the antisense strand siRNA.

4. Computation of GC content for selection of target sequences with preferred GC content (e.g, low to balanced GC).

5. Performing alignment search (e.g., BLAST) to avoid significant homology to other genes in the experimental system.

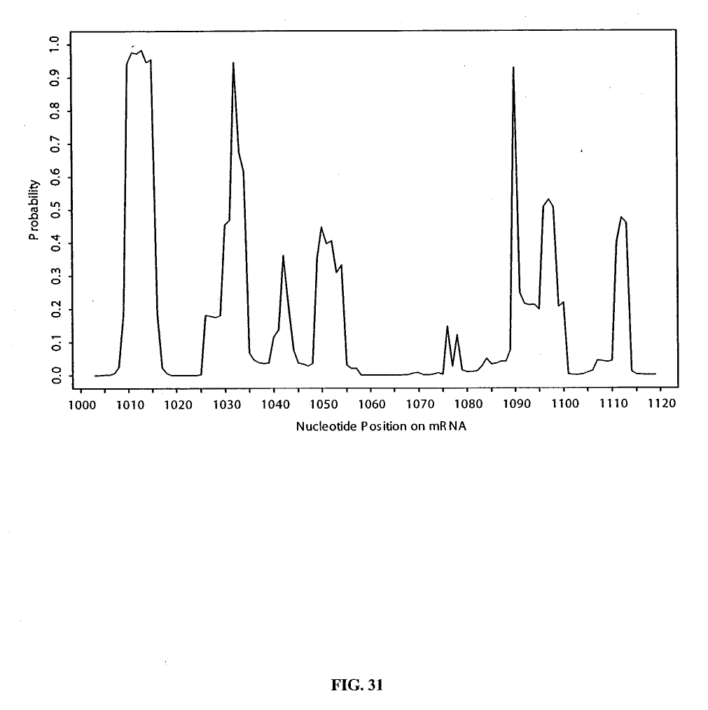

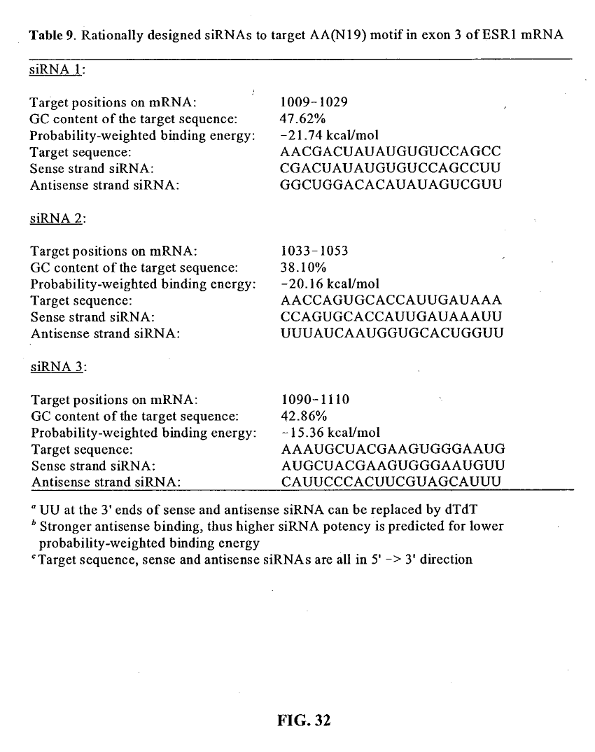

Example of siRNA design. Exon 3 of human estrogen receptor 1 (ESR1, GenBank Accession No. NM_{—}000125) is the region of interest. The entire 6450 nt mRNA of ESR 1 was folded by the sampling algorithm. There is a total of 470 AA(N19) motifs on the mRNA, including 5′ UTR and 3′ UTR. The probability profile for exon 3 is displayed by FIG. 31. There are six AA(N19) motifs within exon 3, and three more with majority of bases within the exon. Three of the nine target sequences are predicted to be well accessible (Table 9 in FIG. 32).

Advantages of Present Invention

Long RNAs may be trapped in locally stable structures. Furthermore, for long-chain RNAs, there are many suboptimal foldings with free energies close to the minimum free energy. It has been a practical problem for antisense experimentalists to select one of the low free energy structures as the basis for antisense design. The suboptimal foldings from mfold do not guarantee a statistically unbiased sample of probable secondary structures. This makes it difficult to assign a statistical measure of confidence for predictions based on these suboptimal foldings. It is possible that each mRNA exists as a population of different structures, and a stochastic approach to accessibility evaluation may be appropriate (Christoffersen et al.). By summarizing a statistical sample of probable structures in a single plot, the probability profile approach of the present invention overcomes these difficulties. The “well-determined” single-stranded regions are revealed by peaks with high probabilities on the profile. Statistical sampling of probable structures provides a suitable means to address these long-standing issues. This is demonstrated by the substantial improvement in predictions over the minimum free energy structure. The sampling method also has the advantage that it does not require the generation of a huge number of all possible structures. For antisense nucleic acid design, the structure sampling algorithm and probability profiling are better suited to the evaluation of target accessibility.

Functional Genomics

The completion of the sequencing of the human genome signals the dawn of a new era in biomedical research. Of the estimated 30,000 ! 40,000 genes in the human genome, definitive functions have been assigned to only a few percent. Functional genomics is concerned with the determination of biological functions for all of the genes and their protein products on a genome-wide scale. Inactivation of a gene is the classical approach to assign a function to a gene in higher organisms. In the post-genomic era, however, gene knockout and mutagenesis, the traditional “gold standard” tools, can no longer keep pace with new sequence information rapidly accumulated from various genome projects. Therefore, antisense nucleic acids that target mRNA have emerged as attractive reverse genetic tools for high throughput functional genomics. Recently, the potential of these RNA-targeting techniques has been demonstrated, through the identification of functional genes by ribozymes in mammalian cells; through chromosome wide phenotypic screening by RNAi in C. elegans; and through genome-wide gene functional alterations by an antisense approach in Candida albicans. The importance of these techniques is further evidenced by the steady increase in the annual number of antisense (FIG. 5 in Ding 2002, attached in the Appendix) and ribozyme papers listed in PubMed, and the recent explosion of RNAi papers in the literature.

Complicated multi-component biological systems can be studied by antisense nucleic acids to independently block the synthesis of each individual protein in the system. Antisense also promises to reveal genetic pathways through expression arrays. By inhibition of protein expression and target mRNA, and through the evaluation of inhibitory effects on expression of genes on DNA arrays, insight will be gained on the gene interaction and regulatory pathways.

Drug Target Validation

Thousands of new potential therapeutic targets have emerged from human genome sequencing. The selection and validation of molecular targets are of paramount importance for drug development in the new millennium. Antisense nucleic acids are important tools for the validation of human therapeutic targets.

High Throughput Applications

DNA expression arrays, which allow the measurement of gene expression patterns of tens of thousands of genes in parallel, have emerged as major high-throughput experimental tools in the post-genomic era. DNA expression arrays can provide important clues to gene function. Genes of similar expression behavior suggest that they are likely to be co-regulated or possibly functionally related. Indeed, statistical clustering analysis has revealed that gene expression data tend to organize genes into functional categories. Genes with unknown function can be assigned tentative functions or a role in a biological process based on the known function of genes in the same cluster.

Single-nucleotide polymorphisms (“SNPs”) promise to propel forward pharmacogenomics, the emerging field concerned with the dissection of the genetic basis of disease and therapeutic response. SNPs enable studies of association between a SNP and risk of a disease or drug response. These associations are valuable for the identification of candidate genes for disease phenotypes.

The eventual determination of the functions of the candidate genes, and confirmation of gene functional predictions based on analysis of DNA expression arrays, will require experimental analysis in a systematic and high throughput fashion to keep pace with the fast growing genome, expression array, and SNP databases. Antisense nucleic acids are well suited for this endeavor. Expression array and SNP databases can provide the basis for high throughput antisense nucleic acid applications to functional genomics and drug target validation.

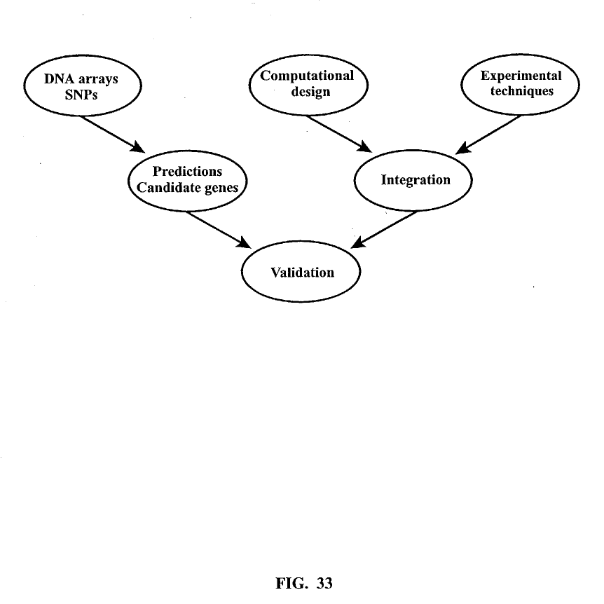

Experimental approaches for finding potent antisense nucleic acids are expensive, time consuming, and laborious, and are usually limited to a region of the target RNA. Published work suggests that, at the very best, only one in eight antisense oligonucleotides is effective. To realize the promise of antisense nucleic acids for high-throughput functional genomics and drug target validation, efficient screening for identifying accessible sites on the target RNA is necessary. This must be based on the combination of a high throughput experimental platform and rational computational method. For example, for the design of antisense oligos, the combinatorial RNA:DNA oligonucleotide array technique appears to be an adequate experimental approach. With labeled transcripts, hybridization intensity can be measured and visualized. However, there are seemingly two practical limitations. First, the number of all possible oligomers up to a preset length is huge for an mRNA. Secondly, large mRNAs can be hampered by their bulky size from approaching the oligomers densely distributed on the array surface. Use of selective oligomers designed by comprehensive computational screening provides a solution. Hence, in accordance with an embodiment of the invention, a strategy of integrating computational predictions and experimental techniques such as oligonucleotide array for a rational, efficient, and comprehensive platform for antisense nucleic acid screening may be used, as shown in FIG. 33.

Folding and Accessibility Prediction for DNA Targets

The focus of the description of the invention has been on RNA targets, however, the algorithms for prediction of secondary structure and target accessibility can be straightforwardly applied to DNA targets by using DNA thermodynamic parameters, such as summarized by SantaLucia.

Design of Oligonucleotide Probes and Molecular Beacons

The folding and accessibility prediction for either RNA or DNA targets are valuable for the design of oligonucleotide probes such as molecular beacons for effective hybridization to the target. Molecular beacons are dual-labeled oligonucleotide probes that are capable of forming a stem-loop structure in the absence of target (Tyagi & Kramer). The loop portion of the molecule is a probe sequence that is complementary to a predetermined sequence in a target nucleic acids. The probes fluoresce only when they hybridize to their complementary targets. When introduced into living cells, these probes may enable the origin, movement and fate of mRNAs to be traced.

Other Applications

Studies of infectious pathogens. Functional studies of genes and their products for CDC high priority pathogens are important for biodefense. For example, for the causative agent of plague, Yersinia pestis, the functions are yet unknown for 1,066 of the 4,012 protein-coding genes (http://www.sanger.ac.uk/Projects/Y_pestis/). In contrast to the RNAi silencing mechanisms that only functions in eukaryotes, antisense oligos and more recently ribozymes have been demonstrated to be effective in bacterial systems. Thus, gene inhibition by antisense oligos or ribozymes are important for applications to high priority pathogens for biodefense.

Studies of small regulatory RNAs. Recently, small non-coding RNAs have gained increasing attention for their broad regulatory functions. In particular, microRNAs (miRNAs) are single-stranded antisense RNAs of 21-22 nt that are believed to target 3′ untranslated regions for mediating negative post-transcriptional regulation. For C. elegans, more than 100 miRNAs have been discovered. However, it is a challenge to identify the particular target for each miRNA. Because the antisense target site is likely to be largely unstructured, the combination of sequence alignment and an analysis of accessibility by probability profiling will constitute a promising strategy for addressing this problem.

Improved structure prediction for homologous RNAs. Improved structure predictions for homologous RNAs, in particular, mRNAs, may be possible by taking advantage of both the statistical sampling paradigm and the potential conservation in structure for sequences of related species available from genome sequencing projects. This will in turn improve the prediction of target accessibility for antisense nucleic acid design.

Algorithm extensions to permit experimental and deterministic constraints. Experimental information on secondary structure can be incorporated into an algorithm to improve predictions by eliminating biochemically invalid structures. Several types of experimental constraints are: a base is paired (partner unknown), a forced base pair, an unpaired base, and an unwanted base pair. These constraints can be extended to consecutive bases or base pairs. Base pairing can also be prohibited between two regions. These constraints are deterministic, because it is implicitly assumed that there is no uncertainty in the assignment of base pairs or unpaired bases. For mathematical algorithms, the constraints can be handled by assigning a large penalty energy (e.g., an unwanted base pair) or a bonus energy (e.g., a forced base pair) in the forward recursions. Similarly, free energies may be adjusted in the calculation of the partition functions to address constraints. The sampling probabilities are adjusted accordingly, such that sampled structures meet the constraints. The bonus energy treatment can be a problem, because large bonuses cause overflows of partition functions. An alternative to assigning a bonus is to penalize all opposite cases. For a base forced to pair, e.g., a large penalty energy can be assigned to the cases of the base being unpaired.

Algorithm extensions to permit experimental and probabilistic constraints. There is often variation in the intensity of the reaction in enzymatic or chemical probing. Weak to very strong enzymatic cuts can be indicated by different levels of intensity on an electrophoretic gel. This probably reflects some heterogeneity in the RNA population as a result of transient intra-molecular interactions and molecular “breathing” of weak base pairs. Another reason for the variability may be the steric hindrance problem due to the bulkiness of RNases. The variability introduces uncertainty in the assignment of base pairs or unpaired bases from the reaction data. A probabilistic approach can address the uncertainty. Assignment of probabilities have been considered for base pairs using enzymatic digestion data in a heuristic matrix method for structure modeling (Quigley et al.). Pooling of information from several reactions by calculating renormalized probabilities have also been considered. The uncertain base pairs and unpaired bases together with their probabilities define what are called probabilistic constraints. A two-step method may be considered to accommodate such constraints. The first step is a “coin flip” step for simulating deterministic constraints by sampling with the probabilities. The collection of outcomes defines a set of deterministic constraints. In step two, a secondary structure is sampled with the algorithm for deterministic constraints. This two-step process is repeated to generate a sample of structures. An alternative is to include probabilities and their corresponding deterministic constraints in a single round of calculation of the partition functions by a possibly a weighting scheme.

Algorithm extensions for H-pseudoknot prediction. A set of parameter estimates for H-pseudoknots, important tertiary structure motifs has been compiled (Gultyaev et al.). This parameter set is based on experimentally and/or phylogenetically proven pseudoknots. An efficient algorithm based on the present invention for H-pseudoknot prediction may take the following steps:

1. Sample a large number of secondary structures with the statistical sampling algorithm.

2. Identify all hairpins for each sampled structure, and predict H-pseudoknots by evaluating stabilities with the parameters for H-pseudoknots.

3. Compute the sampling estimates of probabilities of the predicted H-pseudoknots.

This procedure evaluates stabilities of potential H-pseudoknots after the prediction of an unknotted structure. It has several advantages: (1) a sample simulated by the rigorous sampling algorithm reflects the Boltzmann ensemble of the secondary structures. The resulting predictions of H-pseudoknots are based on an unbiased sample of probable alternatives rather than a single optimal or a few suboptimal structures; (2) the algorithm will be able to incorporate credible free energy estimates for H-pseudoknots and return probabilities of predicted H-pseudoknots for an assessment of confidence in the predictions; (3) because of the fast sampling algorithm, the procedure will be efficient; (4) this approach can be easily extended to predict more general types of pseudoknots when credible parameters are available. The extension only requires the identification of all loop regions in step 2.

Sampling framework for folding of multiple nucleic acids and other type of biomolecules. The sampling approach disclosed in the invention may be applicable to folding of multiple nucleic acids and other type of biomolecules such as proteins, by computing partition functions with energy parameters and sampling molecular conformations. For example, for two nucleic acid molecules, prediction of folding may involve the following basic steps:

1. Calculation of joint partition functions of the two molecules using free energy parameters.

2. Inclusion of molecular concentrations.

3. Sampling of bimolecular conformations using probabilities computed with calculations in step 1 and 2.

A Software for-Statistical Folding and Rational Design of Nucleic Acids

Sfold is a suite of statistical nucleic acid folding software. Sfold currently has four modules with a focus on antisense nucleic acid design: Srna, Soligo, Sribo, and Sirna. Srna offers general features for statistical RNA folding, and Soligo presents tools for target accessibility prediction and the rational design of antisense oligos. Sribo provides both graphical and quantitative tools for target accessibility prediction and the rational design of trans-cleaving ribozymes. It will allow user input of ribozyme type (hammerhead or hairpin), preferred cleavage sequence (e.g., GUC for hammerhead), target RNA, conserved and variable portions of the ribozyme, and possibly other information for user-friendly applications. Sirna offers tools for target accessibility prediction and the rational design of siRNAs for RNA interference. Furthermore, the tools for antisense accessibility are useful for design of oligonucleotides probes such as molecular beacons for nucleic acid hybridization. Version 1.0 of Sfold has been developed, and a Web server for on-line applications will be located at http://www.wadsworth.org/Sfold and/or http://www.bioinfo.rpi.edu/applications/Sfold.

It will thus be seen that the objects set forth above, among those made apparent from the preceding description, are efficiently attained and, because certain changes may be made in carrying out the above method(s) and in the construction(s) set forth without departing from the spirit and scope of the invention, it is intended that all matter contained in the above description and shown in the accompanying drawings shall be interpreted as illustrative and not in a limiting sense.

The present invention may be described by the following numbered paragraphs.

1. A statistical algorithm for generating a sample (of any desired size) of probable secondary structures for a given RNA sequence exactly and rigorously with Boltzmann equilibrium probabilities of RNA secondary structures comprising the steps of:

a) calculating partition functions using latest Turner thermodynamics parameters; and

b) performing random tracebacks using conditional probabilities computed with partition functions.

2. An extension of the algorithm of paragraph 1 to compute probabilities of one or more structural motifs with or without constraints for an RNA molecule comprising the steps of:

a) generation of a sample of probable secondary structures with the algorithm of paragraph 1;

b) estimation of the probability of a structural motif by using the observed frequency of the motif in the sample.

3. An extension of the algorithm of paragraphs 1 or 2 wherein said one or more structural motifs includes one of a helix and a loop.

4. The calculation of Boltzmann-probability-weighted density of states (BPWDOS) and free energy distributions comprising the steps of:

a) generation of a sample of probable secondary structures with the algorithm of paragraphs 1, 2 or 3;

b) calculation and display of BPWDOS, the distribution over free energy intervals for sampled structures (i.e., free energy histogram);

c) calculation and display of the distribution for the probability that the free energy of a structure is within a threshold of the global minimum;

d) calculation and display of the distribution for the probability that the free energy of a structure is within an energy interval.

5. An extension of the algorithm of paragraphs 1, or 2, or 3 to compute probability profiles of single-stranded bases or single-stranded segments of any number of bases for a complete statistical delineation of potential antisense nucleation sites on the entire target RNA comprising the steps of:

a) generating a sample of probable secondary structures with the algorithm of paragraphs 1, or 2, or 3;

b) estimating the probability that a base or a segment of bases of specified length is single-stranded by using the observed frequency in the sample; and

c) repeating above step for all bases or segments on the target RNA for complete profiles.

6. The calculation of a sampling-probability-weighted free energy (ΔG_{nucleation}) for measuring the nucleation potential of the hybridization between an antisense oligo and its target sequence on mRNA and the use of ΔG_{nucleation} and the probability profiles of paragraphs 1, or 2 or 3 for an automated ranking of all antisense oligos of any specified length, and consequently an automated computer selection and design process of antisense oligos. The calculation uses the probabilities from the profiles as weights in the summation of RNA:DNA thermodynamic parameters for the hybrid.

7. The use of the algorithm of paragraphs 1 and the extension of paragraph 2 and/or any index or procedure based on the algorithm or the extension for target prediction, screening and design of antisense oligos for functional genomics, drug target validation and development of antisense therapeutics.

8. The use of the algorithm of paragraph 1 and the extension of paragraph 2 and/or 3 and/or any index or procedure based on the algorithm or the extension for:

a) predicting a potential effective target for a ribozyme of a specified type (e.g., hammerhead, hairpin) with a specified cleavage site (e.g., GUC for hammerhead ribozyme);

b) evaluating the accessibility of the substrate-binding arms of the ribozyme resulted from the predicted target, and the mutual accessibility between the binding arms and the substrate with the probability profiles for the ribozyme and the target RNA; and

c) using the designed ribozymes for functional genomics, drug target validation, and development of ribozymes for human therapeutics.

9. A statistical algorithm for generating a sample (of any desired size) of probable secondary structures for a given DNA sequence based on any set of DNA thermodynamics parameters comprising the steps of:

a) calculating partition functions using DNA thermodynamics parameters;

b) performing random tracebacks using conditional probabilities computed with partition functions.

10. The use of the algorithm of paragraph 1 and/or 2 and/or 3 and the extension of paragraph 4 with RNA or DNA thermodynamics parameters and/or any index or procedure based on the algorithm or the extension for the design of oligonucleotide probes for enhancing signals on nucleic acids hybridization arrays and thus producing higher quality array data for analysis.

11. A method of generating a sample of a predetermined number of probable secondary structures of an RNA sequence, comprising the steps of:

a) generating one or more partition functions of a fragment having one or more bases of the RNA sequence in accordance with a predetermined number of thermodynamics parameters; and

b) generating secondary structures based on tracebacks using conditional probabilities computed with the partition functions.

12. The method of paragraph 11, wherein the thermodynamics parameters include a predetermined number of free energies for basic structural elements.

13. The method of paragraph 11, wherein the thermodynamics parameters include free energies for base pair stacking in a helix.

14. The method of paragraph 11, wherein the partition function generating step generates partition functions for all fragments of the RNA sequence.

15. A method of generating a complete statistical delineation of potential antisense nucleation sites on a target RNA, comprising the steps of:

a) generating a sample of one or more probable secondary structures of an RNA sequence by:

i) generating one or more partition functions of a fragment having one or more bases of the RNA sequence in accordance with a predetermined number of thermodynamics parameters, and

ii) generating secondary structures based on tracebacks using conditional probabilities computed with the partition functions;

b) estimating a probability that a segment of one or more bases on the target RNA is single-stranded in accordance with an observed frequency in the sample; and

c) repeating the estimating step for all bases on the target RNA.

16. A method of determining an antisense oligo of a predetermined length for an antisense nucleation site on a target RNA, comprising the steps of:

a) generating a sample of one or more probable secondary structures of an RNA sequence by:

i) generating one or more partition functions of a fragment having one or more bases of the RNA sequence in accordance with a predetermined number of thermodynamics parameters, and

ii) generating secondary structures based on tracebacks using conditional probabilities computed with the partition functions;

b) estimating a probability that a segment of one or more bases on the target RNA is single-stranded by using an observed frequency in the sample;

c) repeating the estimating step for all bases on the target RNA;

d) identifying a target segment in accordance with the estimated probabilities;

e) determining a base sequence of the target segment; and

f) determining the antisense oligo in accordance with the base sequence.

17. A method of evaluating an antisense oligo for a target RNA, comprising the steps of:

a) generating a sample of one or more probable secondary structures of an RNA sequence by:

i) generating one or more partition functions of a fragment having one or more bases of the RNA sequence in accordance with a predetermined number of thermodynamics parameters, and

ii) generating secondary structures based on tracebacks using conditional probabilities computed with the partition functions;

b) estimating a probability that a segment of one or more bases on the target RNA is single-stranded in accordance with an observed frequency in the sample; and

c) repeating the estimating step for all bases on the target RNA;

d) calculating a sampling-probability-weighted free energy for measuring the nucleation potential of the hybridization between the antisense oligo and the target RNA; and

e) generating an evaluation indicator for the antisense oligo in accordance with the sampling-probability-weighted free energy and the estimated probabilities for the target RNA.

18. The method of paragraph 17, wherein the calculating step includes applying the estimated probabilities as weights in a summation of RNA:DNA thermodynamic parameters for the hybrid.

19. A computer program embodied on a computer-readable medium for generating a sample of a predetermined number of probable secondary structures of an RNA sequence, comprising:

a) an instruction for generating one or more partition functions of a fragment having one or more bases of the RNA sequence in accordance with a predetermined number of thermodynamics parameters; and

b) an instruction for generating secondary structures based on tracebacks using conditional probabilities computed with the partition functions.

20. A computer program embodied on a computer-readable medium for generating a complete statistical delineation of potential antisense nucleation sites on a target RNA, comprising:

a) an instruction for generating a sample of one or more probable secondary structures of an RNA sequence by:

i) generating one or more partition functions of a fragment having one or more bases of the RNA sequence in accordance with a predetermined number of thermodynamics parameters, and

ii) generating secondary structures based on tracebacks using conditional probabilities computed with the partition functions;

b) an instruction for estimating a probability that a segment of one or more bases on the target RNA is single-stranded in accordance with an observed frequency in the sample, wherein

the estimating instruction is repeated for all bases on the target RNA.

21. A computer program embodied on a computer-readable medium for determining an antisense oligo of a predetermined length for an antisense nucleation site on a target RNA, comprising:

a) an instruction for generating a sample of one or more probable secondary structures of an RNA sequence by:

i) generating one or more partition functions of a fragment having one or more bases of the RNA sequence in accordance with a predetermined number of thermodynamics parameters, and

ii) generating secondary structures based on tracebacks using conditional probabilities computed with the partition functions;

b) an instruction for estimating a probability that a segment of one or more bases on the target RNA is single-stranded by using an observed frequency in the sample, said estimating instruction being repeated for all bases on the target RNA;

d) an instruction for identifying a target segment in accordance with the estimated probabilities;

e) an instruction for determining a base sequence of the target segment; and

f) an instruction for determining the antisense oligo in accordance with the base sequence.

22. A computer program embodied on a computer-readable medium for evaluating an antisense oligo for a target RNA, comprising:

a) an instruction for generating a sample of one or more probable secondary structures of an RNA sequence by:

i) generating one or more partition functions of a fragment having one or more bases of the RNA sequence in accordance with a predetermined number of thermodynamics parameters, and

ii) generating secondary structures based on tracebacks using conditional probabilities computed with the partition functions;

b) an instruction for estimating a probability that a segment of one or more bases on the target RNA is single-stranded in accordance with an observed frequency in the sample, said estimating instruction being repeated for all bases on the target RNA;

d) an instruction for calculating a sampling-probability-weighted free energy for measuring the nucleation potential of the hybridization between the antisense oligo and the target RNA; and

c) an instruction for generating an evaluation indicator for the antisense oligo in accordance with the sampling-probability-weighted free energy and the estimated probabilities for the target RNA.

23. A process embodied in an instruction signal of a computing device for generating a sample of a predetermined number of probable secondary structures of an RNA sequence, comprising:

a) an instruction for generating one or more partition functions of a fragment having one or more bases of the RNA sequence in accordance with a predetermined number of thermodynamics parameters; and

b) an instruction for generating secondary structures based on tracebacks using conditional probabilities computed with the partition functions.

24. A process embodied in an instruction signal of a computing device for generating a complete statistical delineation of potential antisense nucleation sites on a target RNA, comprising:

a) an instruction for generating a sample of one or more probable secondary structures of an RNA sequence by:

i) generating one or more partition functions of a fragment having one or more bases of the RNA sequence in accordance with a predetermined number of thermodynamics parameters, and

ii) generating secondary structures based on tracebacks using conditional probabilities computed with the partition functions;

b) an instruction for estimating a probability that a segment of one or more bases on the target RNA is single-stranded in accordance with an observed frequency in the sample, wherein

the estimating instruction is repeated for all bases on the target RNA.

25. A process embodied in an instruction signal of a computing device for determining an antisense oligo of a predetermined length for an antisense nucleation site on a target RNA, comprising:

a) an instruction for generating a sample of one or more probable secondary structures of an RNA sequence by:

i) generating one or more partition functions of a fragment having one or more bases of the RNA sequence in accordance with a predetermined number of thermodynamics parameters, and

ii) generating secondary structures based on tracebacks using conditional probabilities computed with the partition functions;

b) an instruction for estimating a probability that a segment of one or more bases on the target RNA is single-stranded by using an observed frequency in the sample, said estimating instruction being repeated for all bases on the target RNA;

d) an instruction for identifying a target segment in accordance with the estimated probabilities;

e) an instruction for determining a base sequence of the target segment; and

f) an instruction for determining the antisense oligo in accordance with the base sequence.

26. A process embodied in an instruction signal of a computing device for evaluating an antisense oligo for a target RNA, comprising:

a) an instruction for generating a sample of one or more probable secondary structures of an RNA sequence by:

i) generating one or more partition functions of a fragment having one or more bases of the RNA sequence in accordance with a predetermined number of thermodynamics parameters, and

ii) generating secondary structures based on tracebacks using conditional probabilities computed with the partition functions;

b) an instruction for estimating a probability that a segment of one or more bases on the target RNA is single-stranded in accordance with an observed frequency in the sample, said estimating instruction being repeated for all bases on the target RNA;

d) an instruction for calculating a sampling-probability-weighted free energy for measuring the nucleation potential of the hybridization between the antisense oligo and the target RNA; and

e) an instruction for generating an evaluation indicator for the antisense oligo in accordance with the sampling-probability-weighted free energy and the estimated probabilities for the target RNA.

27. A method for the representation and characterization of the Boltzmann ensemble of RNA secondary structures, comprising the steps of:

a) generation of a sample of probable secondary structures with the algorithm of paragraph 1;

b) classification of the sampled structures into classes of similar structures;

c) calculation of the probability for each of the class using the frequency of the class in the sample;

d) display of a class by two-dimensional or equivalent three-dimensional plot for the frequency of base pairs in the class; and

e) computation of the Boltzmann probability of the most probable structure (i.e., the structure with the lowest free energy) in a class as the class representative.

28. A method for the representation and characterization of the Boltzmann ensemble of RNA secondary structures, comprising the steps of:

a) generation of a sample of probable secondary structures with the algorithm of paragraph 1;

b) classification of the sampled structures into classes of similar structures;

c) calculation of the probability for each of the class using the frequency of the class in the sample;

d) display of a class by two-dimensional or equivalent three-dimensional plot for the frequency of base pairs, in the class;

e) computation of the Boltzmann probability of the most probable structure (i.e., the structure with the lowest free energy) in a class as the class representative.

29. A method for generating a mutual accessibility plot for evaluating the potential of RNA:RNA interaction, comprising the steps of:

a) generating probability profile with the algorithm in paragraph 5 for RNA molecule A;

b) generating probability profile with the algorithm in paragraph 5 for RNA molecule B;

c) overlay of the portions of the profiles in a sense:antisense orientation for the region of potential interaction where RNA molecule A and RNA molecule B have complementary bases.

30. A method for target accessibility prediction and the rational design of antisense oligos, comprising the steps of:

a) computation for the construction of the complete probability profile of the target RNA with the algorithm in paragraph 5;

b) selection of accessible sites predicted by high probability peaks on the profile;

c) selection of the antisense oligo of preferred length (e.g., 20 bases) for each accessible site with the strongest probability-weighted-binding energy calculated with RNA/DNA stacking energy parameters;

d) avoidance of three contiguous Gs, a motif known to cause non-specific effects;

e) performing alignment search (e.g., BLAST) to avoid significant homology to other genes in the experimental system.

31. A method for target accessibility prediction and the rational design of trans-cleaving ribozymes, comprising the steps of:

a) computation for the construction of the complete probability profile for the target RNA with the algorithm in paragraph 5;

b) evaluation of accessibility of both the cleavage site (e.g., GUC for hammerhead ribozyme) and its flanking sequences;

c) specification of the bases of the ribozyme binding arms and subsequently the ribozymes for accessible sites;

d) computation of the probability profile for each designed ribozyme with the algorithm in paragraph 5;

e) evaluation of accessibility of the ribozyme binding arms;

f) evaluation of appropriateness of the structure of the catalytic domain of the ribozyme by structure classification for estimating the equilibrium between correct fold and alternatives;

g) evaluation of mutual accessibility between the ribozyme binding arms and their target sequences with the method in paragraph 29.

32. A method for target accessibility prediction and the rational design of siRNAs, comprising the steps of:

a) computation for the construction of the complete probability profile of the target RNA with the algorithm in paragraph 5;

b) selection of accessible sequence (e.g., AA(N19) motifs, where N is any nucleotide) of desired length (e.g., 21-23 nt) on the target;

c) computation of probability-weighted-binding energy using the algorithm in paragraph 7 with RNA:DNA thermodynamic parameters replaced by RNA:RNA stacking energy parameters for the duplex formed between each selected target sequence and the antisense strand siRNA;

d) computation of GC content for selection of target sequences with preferred GC content (e.g., low to balanced GC);

e) performing alignment search (e.g., BLAST) to avoid significant homology to other genes in the experimental system.

33. Frameworks based on the algorithms in paragraphs 1 and/or 5 for applications to studies of infectious pathogens for biodefence, studies of small regulatory RNAs, improved structure prediction for homologous RNAs, algorithm extensions to permit experimental constraints and to predict H-pseudoknots, folding prediction of multiple nucleic acids and other types of biomolecules such as proteins.

34. A software named Sfold for statistical nucleic acid folding, and for target accessibility prediction and the rational design of antisense oligos, trans-cleaving ribozymes, siRNAs and other RNA-targeting molecules, and design of oligonucleotide probes such as molecular beacons.

35. A computer program embodied on a computer-readable medium for target accessibility prediction and the rational design of antisense oligos, comprising:

a) an instruction for computation for the construction of the complete probability profile of the target RNA with the algorithm in paragraph 5;

b) an instruction for election of accessible sites predicted by high probability peaks on the profile;

c) an instruction for selection of the antisense oligo of preferred length (e.g., 20 bases) for each accessible site with the strongest probability-weighted-binding energy calculated with RNA:DNA stacking energy parameters;

d) an instruction for avoidance of three contiguous Gs, a motif known to cause non-specific effects;

e) an instruction for performing alignment search (e.g., BLAST) to avoid significant homology to other genes in the experimental system.

36. A computer program embodied on a computer-readable medium for target accessibility prediction and the rational design of trans-cleaving ribozymes, comprising:

a) an instruction for computation for the construction of the complete probability profile for the target RNA with the algorithm in paragraph 5;

b) an instruction for evaluation of accessibility of both the cleavage site (e.g., GUC for hammerhead ribozyme) and its flanking sequences;

c) an instruction for specification of the bases of the ribozyme binding arms and subsequently the ribozymes for accessible sites;

d) an instruction for computation of the probability profile for each designed ribozyme with the algorithm in paragraph 5;

e) an instruction for evaluation of accessibility of the ribozyme binding arms;

f) an instruction for evaluation of appropriateness of the structure of the catalytic domain of the ribozyme by structure classification for estimating the equilibrium between correct fold and alternatives;

g) an instruction for evaluation of mutual accessibility between the ribozyme binding arms and their target sequences with the method in paragraph 29.

37. A computer program embodied on a computer-readable medium for target accessibility prediction and the rational design of siRNAs, comprising:

a) an instruction for computation for the construction of the complete probability profile of the target RNA with the algorithm in paragraph 5;

b) an instruction for selection of accessible sequence (e.g., AA(N19) motifs, where N is any nucleotide) of desired length (e.g., 21-23 nt) on the target;

c) an instruction for computation of probability-weighted-binding energy using the algorithm in paragraph 6 with RNA:DNA thermodynamic parameters replaced by RNA:RNA stacking energy parameters, for the duplex formed between each selected target sequence and the antisense strand siRNA;

d) an instruction for computation of GC content for selection of target sequences with preferred GC content (e.g., low to balanced GC);

e) an instruction for performing alignment search (e.g., BLAST) to avoid significant homology to other genes in the experimental system.

38. A process embodied in an instruction signal of a computing device for target accessibility prediction and the rational design of antisense oligos, comprising:

a) an instruction for computation for the construction of the complete probability profile of the target RNA with the algorithm in paragraph 5;

b) an instruction for election of accessible sites predicted by high probability peaks on the profile;

c) an instruction for selection of the antisense oligo of preferred length (e.g., 20 bases) for each accessible site with the strongest probability-weighted-binding energy calculated with RNA:DNA stacking energy parameters;

d) an instruction for avoidance of three contiguous Gs, a motif known to cause non-specific effects;

e) an instruction for performing alignment search (e.g., BLAST) to avoid significant homology to other genes in the experimental system.

39. A process embodied in an instruction signal of a computing device for target accessibility prediction and the rational design of trans-cleaving ribozymes, comprising:

a) an instruction for computation for the construction of the complete probability profile for the target RNA with the algorithm in paragraph 5;

b) an instruction for evaluation of accessibility of both the cleavage site (e.g., GUC for hammerhead ribozyme) and its flanking sequences;

c) an instruction for specification of the bases of the ribozyme binding arms and subsequently the ribozymes for accessible sites;

d) an instruction for computation of the probability profile for each designed ribozyme with the algorithm in paragraph 5;

e) an instruction for evaluation of accessibility of the ribozyme binding arms;

f) an instruction for evaluation of appropriateness of the structure of the catalytic domain of the ribozyme by structure classification for estimating the equilibrium between correct fold and alternatives;

g) an instruction for evaluation of mutual accessibility between the ribozyme binding arms and their target sequences with the method in paragraph 29.

40. A process embodied in an instruction signal of a computing device for target accessibility prediction and the rational design of siRNAs, comprising:

a) an instruction for computation for the construction of the complete probability profile of the target RNA with the algorithm in paragraph 5;

b) an instruction for selection of accessible sequence (e.g., AA(N19) motifs, where N is any nucleotide) of desired length (e.g., 21-23 nt) on the target;

c) an instruction for computation of probability-weighted-binding energy using the algorithm in paragraph 6 with RNA:DNA thermodynamic parameters replaced by RNA:RNA stacking energy parameters, for the duplex formed between each selected target sequence and the antisense strand siRNA;

d) an instruction for computation of GC content for selection of target sequences with preferred GC content (e.g., low to balanced GC);

e) an instruction for performing alignment search (e.g., BLAST) to avoid significant homology to other genes in the experimental system.

41. The calculation of a sampling-probability-weighted binding energy (ΔG_{nucleation}) for measuring the nucleation potential of the hybridization between an antisense oligo and its target sequence on RNA. The calculation uses the probabilities on the profiles from paragraph 6 as weights in the summation of RNA:DNA thermodynamic parameters for the hybrid.

42. The use of the algorithm of paragraph 1 and the extension of paragraph 2 and/or any index or procedure based on the algorithm or the extension for target prediction, screening and design of antisense nucleic acids for functional genomics, drug target validation and development of RNA-targeting therapeutics.

The invention further comprehends the transmission of information, e.g., antisense or ribozyme or siRNA information, target prediction information, information from screening and/or design of antisense nucleic acids, e.g., as to functional genomics, drug target validation and development of RNA-targeting therapeutics, information on the design of oligonucleotide probes (e,g., molecular beacons), for instance for enhancing signals on nucleic acids hybridization arrays and thus producing higher quality array data for analysis, from any of the herein methods, algorithms, or applications thereof, for example, transmission via a global communications network or the internet, e.g., via Web site posting, such as by subscription or select or secure access thereto and/or via email and/or via telephone, IR, radio or television other frequency signal, and/or via electronic signals over cable and/or satellite transmission and/or via transmission of disks, cds, computers, hard drives, or other apparatus containing the information in electronic form, and/or transmission of written forms of the information, e.g., via facsimile transmission and the like. Thus, the invention comprehends a user performing methods or using algorithms according to the invention and transmitting information therefrom; for instance, to one or more parties who then further utilize some or all of the data or information, e.g., in the manufacture of products, such as therapeutics, antisense nucleic acids, probes, assays, etc. The invention also comprehends disks, cds, computers, or other apparatus or means for storing or receiving or transmitting data or information containing information from methods and/or use of algorithms of the invention.

Thus, the invention comprehends a method for transmitting information comprising performing a method as discussed herein and transmitting a result thereof.

The invention also comprehends a method for target prediction, or for screening or designing of antisense oligos, trans-cleaving ribozyme or siRNAs, or for performing functional genomics, or for drug target validation, or for development of antisense therapeutics, or for the design of oligonucleotide probes (e.g., molecular beacons), or for enhancing signals on nucleic acids hybridization arrays, or for producing higher quality array data, comprising performing a method as herein discussed or using the algorithm as herein discussed. A result or results from the method or use of the algorithm may be correlated to target prediction, or screening or designing of antisense nucleic acids, or performing functional genomics, or drug target validation, or development of RNA-targeting therapeutics, or the design of oligonucleotide probes (e.g., molecular beacons), or enhancing signals on nucleic acids hybridization arrays, or producing higher quality array data.

The invention further comprehends a method for transmitting information for target prediction, or for screening or designing of antisense nucleic acids, or for performing functional genomics, or for drug target validation, or for development of antisense nucleic acids as therapeutics, or for the design of oligonucleotide probes (e.g., molecular beacons), or for enhancing signals on nucleic acids hybridization arrays, or for producing higher quality array data, comprising performing a method as herein discussed or using the algorithm as herein discussed, and transmitting a result thereof. A result or results may be correlated to target prediction, or screening or designing of antisense nucleic acids, or performing functional genomics, or drug target validation, or development of RNA-targeting therapeutics, or the design of oligonucleotide probes (e.g., molecular beacons), or enhancing signals on nucleic acids hybridization arrays, or producing higher quality array data. Advantageously information transmission is via electronic means, e.g., via email or the internet.

Further still, the invention comprehends methods of doing business comprising performing some or all of a herein method or use of a herein algorithm, and communicating or transmitting or divulging a result or the results thereof, advantageously in exchange for compensation, e.g., a fee. Advantageously the communicating, transmitting or divulging is via electronic means, e.g., via internet or email, or by any other transmission means herein discussed.

Thus, a first party, “client” can request information, e.g., via any of the herein mentioned transmission means—either previously prepared information or information specially ordered as to a particular nucleic acid molecule—such as, for example, for or on target prediction or for or on identification of accessible sites on target RNA for gene down-regulation, or for or on identification of single-stranded regions in the secondary structure of a nucleic acid molecule, or for or on screening or designing of antisense oligos or trans-ribozymes or siRNAs, or for or on performing functional genomics, or for or on drug target validation, or for or on development of RNA-targeting therapeutics, or for or on the design of oligonucleotide probes (e.g., molecular beacons), or for or on enhancing signals on nucleic acids hybridization arrays, or for or on producing higher quality array data, of a second party, “vendor”, e.g., requesting information via electronic means such as via internet (for instance request typed into website) or via email, and the vendor can transmit that information, e.g., via any of the transmission means herein mentioned, advantageously via electronic means, such as internet (for instance secure or subscription or select access website) or email: the information can come from performing some or all of a herein method or use of a herein algorithm in response to the request, or from performing some or all of a herein method or use of a herein algorithm, and generating a library of information from performing some or all of a herein method or use of a herein algorithm and meeting the request can then be allowing the client access to the library or selecting data from the library that is responsive to the request.

Accordingly, the invention even further comprehends collections of information, e,g., in electronic form (such as forms of transmission discussed above), from performing a herein method using a herein or portion thereof or using a herein algorithm or performing some or all of a herein method or use of a herein algorithm.

And the invention comprehends linked or networked computers sharing and/or transmitting information from performing a herein method using a herein or portion thereof or using a herein algorithm or performing some or all of a herein method or use of a herein algorithm, such as a server or host computer containing such information and computer or computers, either on the same premises as the server or host computer or remotely situated accessing that information, whereby “transmission” can include the linking of such computers and the access to the information by the remote computer.

It will thus be seen that the objects set forth above, among those made apparent from the preceding description, are efficiently attained and, because certain changes may be made in carrying out the above method(s) and in the construction(s) set forth without departing from the spirit and scope of the invention, it is intended that all matter contained in the above description and shown in the accompanying drawings shall be interpreted as illustrative and not in a limiting sense.

What is claimed is:

1. A method of generating a sample of a predetermined number of probable secondary structures of an RNA sequence, comprising the steps of:
a) generating one or more partition functions of a fragment having one or more bases of the RNA sequence in accordance with a predetermined number of thermodynamics parameters; and
b) generating secondary structures based on tracebacks using conditional probabilities computed with the partition function.

2. The method of claim 1, wherein the thermodynamics parameters include a predetermined number of free energies for basic structural elements.

3. The method of claim 1, wherein the thermodynamics parameters include free energies for base pair stacking in a helix.

4. The method of claim 1, wherein the partition function generating step generates partition functions for all fragments of the RNA sequence.

5. A method of generating a probability profile for predicting an accessible site on a target RNA for interaction with a biomolecule, comprising the steps of:
a) generating a sample of one or more probable secondary structures of an RNA sequence by:
i) generating one or more partition functions of a fragment having one or more bases of the RNA sequence in accordance with a predetermined number of thermodynamics parameters, and
ii) generating secondary structures based on tracebacks using conditional probabilities computed with the partition functions;
b) estimating a probability that a segment of one or more bases on the target RNA is single-stranded in accordance with an observed frequency in the sample; and
c) repeating the estimating step for all segments on the target RNA.

6. A method of determining an antisense oligo of a predetermined length for an antisense nucleation site on a target RNA, comprising the steps of:
a) generating a sample of one or more probable secondary structures of an RNA sequence by:
i) generating one or more partition functions of a fragment having one or more bases of the RNA sequence in accordance with a predetermined number of thermodynamics parameters, and
ii) generating secondary structures based on tracebacks using conditional probabilities computed with the partition functions;
b) estimating a probability that a segment of one or more bases on the target RNA is single-stranded by using an observed frequency in the sample;
c) repeating the estimating step for all segments on the target RNA;
d) identifying a target segment in accordance with the estimated probabilities;
e) determining a base sequence of the target segment; and
f) determining the antisense oligo in accordance with the base sequence.

7. A method of evaluating an antisense oligo for a target RNA, comprising the steps of:
a) generating a sample of one or more probable secondary structures of an RNA sequence by:
i) generating one or more partition functions of a fragment having one or more bases of the RNA sequence in accordance with a predetermined number of thermodynamics parameters, and
ii) generating secondary structures based on tracebacks using conditional probabilities computed with the partition functions;
b) estimating a probability that a segment of one or more bases on the target RNA is single-stranded in accordance with an observed frequency in the sample; and
c) repeating the estimating step for all segments on the target RNA;
d) calculating a sampling-probability-weighted binding energy for measuring a nucleation potential of a hybridization between the antisense oligo and the target RNA; and
e) generating an evaluation indicator for the antisense oligo in accordance with the sampling-probability-weighted binding energy and the estimated probabilities for the target RNA.

8. The method of claim 7, wherein the calculating step includes applying the estimated probabilities as weights in a summation of RNA:DNA thermodynamic parameters for the hybrid.

9. A computer program embodied on a computer-readable medium for generating a sample of a predetermined number of probable secondary structures of an RNA sequence, comprising:
a) an instruction for generating one or more partition functions of a fragment having one or more bases of the RNA sequence in accordance with a predetermined number of thermodynamics parameters; and
b) an instruction for generating secondary structures based on tracebacks using conditional probabilities computed with the partition function.

10. A computer program embodied on a computer-readable medium for generating a probability profile for predicting an accessible site on a target RNA for interaction with a biomolecule, comprising:
a) an instruction for generating a sample of one or more probable secondary structures of an RNA sequence by:
i) generating one or more partition functions of a fragment having one or more bases of the RNA sequence in accordance with a predetermined number of thermodynamics parameters, and
ii) generating secondary structures based on tracebacks using conditional probabilities computed with the partition functions;
b) an instruction for estimating a probability that a segment of one or more bases on the target RNA is single-stranded in accordance with an observed frequency in the sample, wherein the estimating instruction is repeated for all segments on the target RNA.

11. A computer program embodied on a computer-readable medium for determining an antisense oligo of a predetermined length for an antisense nucleation site on a target RNA, comprising:
a) an instruction for generating a sample of one or more probable secondary structures of an RNA sequence by:
i) generating one or more partition functions of a fragment having one or more bases of the RNA sequence in accordance with a predetermined number of thermodynamics parameters, and
ii) generating secondary structures based on tracebacks using conditional probabilities computed with the partition functions;
b) an instruction for estimating a probability that a segment of one or more bases on the target RNA is single-stranded by using an observed frequency in the sample, said estimating instruction being repeated for all segments on the target RNA;
c) an instruction for identifying a target segment in accordance with the estimated probabilities;
d) an instruction for determining a base sequence of the target segment; and
e) an instruction for determining the antisense oligo in accordance with the base sequence.

12. A computer program embodied on a computer-readable medium for evaluating an antisense oligo for a target RNA, comprising:
a) an instruction for generating a sample of one or more probable secondary structures of an RNA sequence by:
i) generating one or more partition functions of a fragment having one or more bases of the RNA sequence in accordance with a predetermined number of thermodynamics parameters, and
ii) generating secondary structures based on tracebacks using conditional probabilities computed with the partition functions;
b) an instruction for estimating a probability that a segment of one or more bases on the target RNA is single-stranded in accordance with an observed frequency in the sample, said estimating instruction being repeated for all bases on the target RNA;
c) an instruction for calculating a sampling-probability-weighted free energy for measuring a nucleation potential of a hybridization between the antisense oligo and the target RNA; and
d) an instruction for generating an evaluation indicator for the antisense oligo in accordance with the sampling-probability-weighted binding energy and the estimated probabilities for the target RNA.

13. A process embodied in an instruction signal of a computing device for generating a sample of a predetermined number of probable secondary structures of an RNA sequence, comprising:
a) an instruction for generating one or more partition functions of a fragment having one or more bases of the RNA sequence in accordance with a predetermined number of thermodynamics parameters; and
b) an instruction for generating secondary structures based on tracebacks using conditional probabilities computed with the partition functions.

14. A process embodied in an instruction signal of a computing device for generating a probability profile for predicting an accessible site on a target RNA for interaction with a biomolecule, comprising:
a) an instruction for generating a sample of one or more probable secondary structures of an RNA sequence by:
i) generating one or more partition functions of a fragment having one or more bases of the RNA sequence in accordance with a predetermined number of thermodynamics parameters, and
ii) generating secondary structures based on tracebacks using conditional probabilities computed with the partition functions;
b) an instruction for estimating a probability that a segment of one or more bases on the target RNA is single-stranded in accordance with an observed frequency in the sample, wherein the estimating instruction is repeated for all segments on the target RNA.

15. A process embodied in an instruction signal of a computing device for determining an antisense oligo of a predetermined length for an antisense nucleation site on a target RNA, comprising:
a) an instruction for generating a sample of one or more probable secondary structures of an RNA sequence by:
i) generating one or more partition functions of a fragment having one or more bases of the RNA sequence in accordance with a predetermined number of thermodynamics parameters, and
ii) generating secondary structures based on tracebacks using conditional probabilities computed with the partition functions;
b) an instruction for estimating a probability that a segment of one or more bases on the target RNA is single-stranded by using an observed frequency in the sample, said estimating instruction being repeated for all segments on the target RNA;
c) an instruction for identifying a target segment in accordance with the estimated probabilities;
d) an instruction for determining a base sequence of the target segment; and
e) an instruction for determining the antisense oligo in accordance with the base sequence.

16. A process embodied in an instruction signal of a computing device for evaluating an antisense oligo for a target RNA, comprising:
a) an instruction for generating a sample of one or more probable secondary structures of an RNA sequence by:
i) generating one or more partition functions of a fragment having one or more bases of the RNA sequence in accordance with a predetermined number of thermodynamics parameters, and
ii) generating secondary structures based on tracebacks using conditional probabilities computed with the partition functions;
b) an instruction for estimating a probability that a segment of one or more bases on the target RNA is single-stranded in accordance with an observed frequency in the sample, said estimating instruction being repeated for all segments on the target RNA;
c) an instruction for calculating a sampling-probability-weighted free energy for measuring a nucleation potential of a hybridization between the antisense oligo and the target RNA; and
d) an instruction for generating an evaluation indicator for the antisense oligo in accordance with the sampling-probability-weighted free energy and the estimated probabilities for the target RNA.

17. A method for transmitting information comprising performing a method as claimed in any one of claims 1-8 and transmitting a result thereof.

18. A method for target prediction or for identification of effective sites on target RNA for gene down-regulation, or for identification of single-stranded regions in the secondary structure of an mRNA or viral RNA, or for screening or designing of antisense oligos or ribozymes, or for performing functional genomics, or for drug target validation, or for development of antisense therapeutics, or for the design of oligonucleotide probes, or for enhancing signals on nucleic acids hybridization arrays, or for producing higher quality array data, comprising performing a method as claimed in any one of claims 1-8.

19. A method for transmitting information for or on target prediction or for or on identification of effective sites on target RNA for gene down-regulation, or for or on identification of single-stranded regions in the secondary structure of an mRNA or viral RNA, or for or on screening or designing of antisense oligos or ribozymes, or for or on performing functional genomics, or for or on drug target validation, or for or on development of antisense therapeutics, or for or on the design of oligonucleotide probes, or for or on enhancing signals on nucleic acids hybridization arrays, or for or on producing higher quality array data, comprising performing a method as claimed in any one of claims 1-8, and transmitting a result thereof.

20. The method of claim 19 wherein the transmitting is via email or the internet.
